(19) United States
(12) Patent Application Publication
(10) Pub. No.: US 2011/0105514 A1
(43) Pub. Date: May 5, 2011

(54) 5,6,7,8-TETRAHYDRO-IMIDAZO[1,5-A]PYRAZINE COMPOUNDS

(76) Inventors: Hamed Aissaoui, Pulversheim (FR); Christoph Boss, Allschwil (CH); Ralf Koberstein, Lorrach (DE); Romain Siegrist, Allschwil (CH); Thierry Sifferlen, Wentzwiller (FR)

(21) Appl. No.: 13/000,201

(22) PCT Filed: Jun. 24, 2009
(86) PCT No.: PCT/IB2009/052701

§ 371 Date: Dec. 20, 2010

(30) Foreign Application Priority Data
Jun. 25, 2008 (IB) PCT/IB2008/052549

(51) Int. Cl. A61K 31/4985; C07D 487/04; A61P 25/00; A61P 25/30

(52) U.S. Cl. 514/249; 544/349

(57) ABSTRACT

The invention relates to 5,6,7,8-tetrahydro-imidazo[1,5-a]pyrazine derivatives of formula (I) wherein R¹, R², R³, and R⁴ are as described n the description, to salts, especially pharmaceutically acceptable salts thereof, and to the use of such compounds as medicaments; especially as orexin receptor antagonists.

The present invention relates to novel 5,6,7,8-tetrahydro-imidazo[1,5-a]pyrazine compounds of formula (I) and their use as pharmaceuticals. The invention also concerns related aspects including processes for the preparation of the compounds, pharmaceutical compositions containing one or more compounds of formula (I), and especially their use as orexin receptor antagonists.

Orexins (orexin A or OX-A and orexin B or OX-B) are novel neuropeptides found in 1998 by two research groups, orexin A is a 33 amino acid peptide and orexin B is a 28 amino acid peptide (Sakurai T. et al., Cell, 1998, 92, 573-585). Orexins are produced in discrete neurons of the lateral hypothalamus and bind to the G-protein-coupled receptors (OX₁ and OX₂ receptors). The orexin-1 receptor (OX₁) is selective for OX-A, and the orexin-2 receptor (OX₂) is capable to bind OX-A as well as OX-B. Orexins are found to stimulate food consumption in rats suggesting a physiological role for these peptides as mediators in the central feedback mechanism that regulates feeding behaviour (Sakurai T. et al., Cell, 1998, 92, 573-585). On the other hand, it was also observed that orexins regulate states of sleep and wakefulness opening potentially novel therapeutic approaches to narcolepsy as well as insomnia and other sleep disorders (Chemelli R. M. et al., Cell, 1999, 98, 437-451). Furthermore, in vitro and in vivo evidence for a critical role of orexin signaling in the ventral tegmental area in neural plasticity relevant to addiction has been published (S. L. Borgland et al. Neuron, 2006, 49, 589-601).

Thus, orexin receptors may have numerous implications in pathologies as known from the literature, such as dysthymic, mood, psychotic and anxiety disorders; diabetes and appetite, taste, eating, or drinking disorders; hypothalamic diseases; disturbed biological and circadian rhythms; sleep disturbances associated with diseases such as neurological disorders, neuropathic pain and restless leg syndrome; insomnias related to psychiatric disorders; sleep apnea; narcolepsy; idiopathic insomnias; parasomnias; benign prostatic hypertrophy; all dementias and cognitive dysfunctions in the healthy population and in psychiatric and neurologic disorders; and other diseases related to general orexin system dysfunctions. The compound (2R)-2-{(1S)-6,7-dimethoxy-1-[2-(4-trifluoromethyl-phenyl)-ethyl]-3,4-dihydro-1H-isoquinolin-2-yl}-N-methyl-2-phenyl-acetamide (WO2005/118548) is currently in clinical development for primary insomnia. In the rat, the compound has been shown for example to decrease alertness, characterized by decreases in both active wake and locomotion; and to dose-dependently increase the time spent in both REM and NREM sleep (F. Jenck et al., Nature Medicine 2007, 13, 150-155). The compound has also been shown to enhance memory function in a rat model (WO2007/105177) and is also active in a rat model of post-traumatic stress disorder (WO2009/047723).

The present invention provides novel substituted 5,6,7,8-tetrahydro-imidazo[1,5-a]pyrazine derivatives, which are non-peptide antagonists of human orexin OX₁ and/or OX₂ receptors and, thus, of potential use in the treatment of diseases related to the orexin system, especially comprising all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders. In particular these compounds are of potential use in the treatment of eating disorders, drinking disorders, sleep disorders, or cognitive dysfunctions in psychiatric and neurologic disorders.

1) A first aspect of the invention relates to 5,6,7,8-tetrahydro-imidazo[1,5-a]pyrazine derivatives of formula (I),

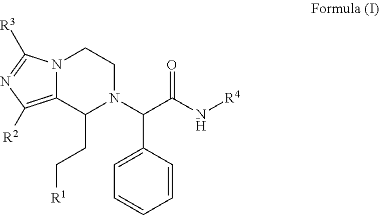

wherein
R⁴ represents (C₁₋₄)alkyl; and
R¹, R², and R³ represent one of the following combinations:
R³ represents cyclopropyl;
R² represents halogen, trifluoromethyl, (C₁₋₄)alkyl, or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
wherein the above combination may be further characterized by the following sub-embodiments: in one sub-embodiment R² represents halogen, trifluoromethyl, or (C₁₋₄)alkyl (especially (C₁₋₄)alkyl or trifluoromethyl); in another sub-embodiment R² represents halogen; in another sub-embodiment R² represents trifluoromethyl; in another sub-embodiment R² represents (C₁₋₄)alkyl; and in yet another sub-embodiment R² represents vinyl;
or
R³ represents (C₃₋₆)cycloalkyl-(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —SO₂—(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents halogen, trifluoromethyl, or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
wherein the above combination may be further characterized by the following sub-embodiments: in one sub-embodiment R² represents halogen or trifluoromethyl; in another sub-embodiment R² represents halogen; in another sub-embodiment R² represents trifluoromethyl; and in yet another sub-embodiment R² represents vinyl;
or
R³ represents (C₁₋₄)alkyl;
R² represents —S{O}ₙ—(C₁₋₄)alkyl, wherein n represents the integer 0 or 2; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkoxy;
R² represents trifluoromethyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents trifluoromethyl;
R² represents (C₁₋₄)alkyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl.

In the present description the term “halogen” means fluorine, chlorine, bromine or iodine.

For the substituent R¹, the term “halogen” means fluorine, chlorine, or bromine, and preferably fluorine or chlorine. More preferred the term “halogen” means fluorine.

For the substituent R², the term “halogen” means fluorine, chlorine, bromine or iodine, and preferably chlorine.

For the substituent R³, the term “halogen” means fluorine, chlorine, bromine or iodine, and preferably chlorine.

The term “(C₁₋₄)alkyl”, alone or in combination, means a straight-chain or branched-chain saturated alkyl group with 1 to 4 carbon atoms. Examples of (C₁₋₄)alkyl groups are methyl, ethyl, propyl, isopropyl, n-butyl, isobutyl, sec.-butyl or tert.-butyl. Preferred are methyl and ethyl. The term “(C₁₋₂)alkyl” means a methyl or ethyl group.

For the substituent R¹, the term “(C₁₋₄)alkyl” means preferably methyl or ethyl, especially methyl.

For the substituent R², the term “(C₁₋₄)alkyl” means preferably methyl or ethyl, especially methyl.

For the substituent R³, the term “(C₁₋₄)alkyl” means preferably methyl, ethyl, n-propyl or isopropyl. More preferred the term “(C₁₋₄)alkyl” means methyl or ethyl. In a sub-embodiment, the term “(C₁₋₄)alkyl” means ethyl. In another sub-embodiment, the term “(C₁₋₄)alkyl” means methyl.

For the substituent R⁴, the term “(C₁₋₄)alkyl” means preferably methyl.

Examples of “—S{O}ₙ—(C₁₋₄)alkyl, wherein n represents the integer 0 or 2” groups are, in case n represents O, —S—(C₁₋₄)alkyl groups such as —S—CH₃ (methylthio-); and, in case n represents 2, —SO₂—(C₁₋₄)alkyl groups such as —SO₂—CH₃ (methanesulfonyl-).

The term “(C₃₋₆)cycloalkyl-(C₁₋₄)alkyl”, alone or in combination, means a group of the formula (C₃₋₆)cycloalkyl-(C₁₋₄)alkyl- in which the term (C₃₋₆)cycloalkyl means a monocyclic saturated alkyl group with 3 to 6 carbon atoms, and the term “(C₁₋₄)alkyl” has the previously given significance. Examples of (C₃₋₆)cycloalkyl-(C₁₋₄)alkyl groups are cyclopropyl-methyl, cyclobutyl-methyl, cyclopentyl-methyl, cyclohexyl-methyl, and cyclopropyl-ethyl. Preferred is cyclopropyl-methyl.

The term “(C₁₋₄)alkoxy”, alone or in combination, means a group of the formula (C₁₋₄)alkyl-O— in which the term “(C₁₋₄)alkyl” has the previously given significance. Examples are methoxy, ethoxy, n-propoxy, isopropoxy, n-butoxy, isobutoxy, sec.-butoxy or tert.-butoxy. Preferred are methoxy and ethoxy.

For the substituent R¹, the term “(C₁₋₄)alkoxy” means preferably methoxy.

For the substituent R³, the term “(C₁₋₄)alkoxy” means preferably methoxy or ethoxy; more preferred is ethoxy.

In case R¹ represents a “phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl”, the subtituents preferably are independently selected from the group consisting of (C₁₋₄)alkyl, (C₁₋₄)alkoxy, halogen, difluoromethoxy, trifluoromethoxy and trifluoromethyl (especially the substituents are independently selected from the group consisting of halogen, difluoromethoxy, trifluoromethoxy and trifluoromethyl). Examples of such groups as used for the substituent R¹ are difluoromethoxy-phenyl (e.g. 4-difluoromethoxy-phenyl, 3-difluoromethoxy-phenyl), trifluoromethyl-phenyl (e.g. 4-trifluoromethyl-phenyl, 3-trifluoromethyl-phenyl), trifluoromethoxy-phenyl (e.g. 4-trifluoromethoxy-phenyl, 3-trifluoromethoxy-phenyl), fluoro-difluoromethoxy-phenyl (e.g. 3-fluoro-4-difluoromethoxy-phenyl, 2-fluoro-4-difluoromethoxy-phenyl, 2-fluoro-3-difluoromethoxy-phenyl, 4-fluoro-3-difluoromethoxy-phenyl, 5-fluoro-3-difluoromethoxy-phenyl, 6-fluoro-3-difluoromethoxy-phenyl), fluoro-trifluoromethyl-phenyl (e.g. 3-fluoro-4-trifluoromethyl-phenyl, 2-fluoro-4-trifluoromethyl-phenyl, 4-fluoro-3-trifluoromethyl-phenyl), fluoro-trifluoromethoxy-phenyl (e.g. 3-fluoro-4-trifluoromethoxy-phenyl, 2-fluoro-4-trifluoromethoxy-phenyl, 4-fluoro-3-trifluoromethoxy-phenyl), chloro-phenyl (e.g. 3-chloro-phenyl and 4-chloro-phenyl), methyl-phenyl (e.g. 3-methyl-phenyl, 4-methyl-phenyl), cyano-phenyl (e.g. 4-cyano-phenyl), dimethyl-phenyl (e.g. 2,3-dimethyl-phenyl, 2,4-dimethyl-phenyl, 3,4-dimethyl-phenyl), methoxy-phenyl (e.g. 3-methoxy-phenyl, 4-methoxy-phenyl), dimethoxy-phenyl (e.g. 2,5-dimethoxy-phenyl, 2,4-dimethoxy-phenyl), fluoro-methoxy-phenyl (e.g. 3-fluoro-4-methoxy-phenyl), dichloro-phenyl (e.g. 2,4-dichloro-phenyl), difluoro-phenyl (e.g. 3,4-difluoro-phenyl), fluoro-methyl-phenyl (e.g. 3-fluoro-4-methyl-phenyl), chloro-trifluoromethyl-phenyl (e.g. 3-chloro-4-trifluoromethyl-phenyl), difluoro-methyl-phenyl (e.g. 3,5-difluoro-4-methyl-phenyl, 2,4-difluoro-3-methyl-phenyl), difluoro-methoxy-phenyl (e.g. 3,5-difluoro-4-methoxy-phenyl, 2,3-difluoro-4-methoxy-phenyl, 2,5-difluoro-4-methoxy-phenyl), trifluoro-phenyl (e.g. 2,3,5-trifluoro-phenyl, 3,4,5-trifluoro-phenyl), chloro-fluoro-phenyl (e.g. 4-chloro-3-fluoro-phenyl), chloro-difluoro-phenyl (e.g. 4-chloro-3,5-difluoro-phenyl), difluoro-difluoromethoxy-phenyl (e.g. 2,3-difluoro-4-difluoromethoxy-phenyl, 2,6-difluoro-4-difluoromethoxy-phenyl, 2,5-difluoro-4-difluoromethoxy-phenyl, 3,5-difluoro-4-difluoromethoxy-phenyl, 2,4-difluoro-3-difluoromethoxy-phenyl, 2,5-difluoro-3-difluoromethoxy-phenyl, 2,6-difluoro-3-difluoromethoxy-phenyl, 4,5-difluoro-3-difluoromethoxy-phenyl), difluoro-trifluoromethyl-phenyl (e.g. 3,5-difluoro-4-trifluoromethyl-phenyl, 2,3-difluoro-4-trifluoromethyl-phenyl, 2,5-difluoro-4-trifluoromethyl-phenyl), difluoro-trifluoromethoxy-phenyl (e.g. 3,5-difluoro-4-trifluoromethoxy-phenyl, 2,3-difluoro-4-trifluoromethoxy-phenyl, 2,5-difluoro-4-trifluoromethoxy-phenyl). In a sub-embodiment, preferred are difluoromethoxy-phenyl, trifluoromethyl-phenyl, fluoro-difluoromethoxy-phenyl, fluoro-trifluoromethyl-phenyl, fluoro-trifluoromethoxy-phenyl, chloro-fluoro-phenyl, and difluoro-trifluoromethyl-phenyl. In another embodiment, preferably, the above groups are phenyl groups, which are mono-, di-, or tri-substituted, wherein one substituent is difluoromethoxy, trifluoromethoxy or trifluoromethyl (especially in position 3 or 4; in a sub-embodiment in position 3; in another sub-embodiment in position 4), and the remaining substituents (if present) are fluorine. Preferred examples of such groups are 4-difluoromethoxy-phenyl, 3-difluoromethoxy-phenyl, 4-trifluoromethyl-phenyl, 3-trifluoromethyl-phenyl, 4-trifluoromethoxy-phenyl, 3-trifluoromethoxy-phenyl, 3-fluoro-4-difluoromethoxy-phenyl, 2-fluoro-4-difluoromethoxy-phenyl, 2-fluoro-3-difluoromethoxy-phenyl, 4-fluoro-3-difluoromethoxy-phenyl, 5-fluoro-3-difluoromethoxy-phenyl, 6-fluoro-3-difluoromethoxy-phenyl, 3-fluoro-4-trifluoromethyl-phenyl, 2-fluoro-4-trifluoromethyl-phenyl, 4-fluoro-3-trifluoromethyl-phenyl, 3-fluoro-4-trifluoromethoxy-phenyl, 2-fluoro-4-trifluoromethoxy-phenyl, 4-fluoro-3-trifluoromethoxy-phenyl, 2,3-difluoro-4-difluoromethoxy-phenyl, 2,6-difluoro-4-difluoromethoxy-phenyl, 2,5-difluoro-4-difluoromethoxy-phenyl, 3,5-difluoro-4-difluoromethoxy-phenyl, 2,4-difluoro-3-difluoromethoxy-phenyl, 2,5-difluoro-3-difluoromethoxy-phenyl, 2,6-difluoro-3-difluoromethoxy-phenyl, 4,5-difluoro-3-difluoromethoxy-phenyl, 3,5-difluoro-4-trifluoromethyl-phenyl, 2,3-difluoro-4-trifluoromethyl-phenyl, 2,5-difluoro-4-trifluoromethyl-phenyl, 3,5-difluoro-4-trifluoromethoxy-phenyl, 2,3-difluoro-4-trifluoromethoxy-phenyl, and 2,5-difluoro-4-trifluoromethoxy-phenyl. In a sub-embodiment, preferred examples of such groups are 4-difluoromethoxy-phenyl, 3-difluoromethoxy-phenyl, 4-trifluoromethyl-phenyl, 3-fluoro-4-difluoromethoxy-phenyl, 3-fluoro-4-trifluoromethyl-phenyl, 4-fluoro-3-trifluoromethyl-phenyl, 3-fluoro-4-trifluoromethoxy-phenyl, and 2,3-difluoro-4-trifluoromethyl-phenyl.

In case R¹ represents a “phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl”, the remaining substituents (if present) preferably are independently selected from the group consisting of (C₁₋₄)alkyl, (C₁₋₄)alkoxy, halogen, difluoromethoxy, trifluoromethoxy and trifluoromethyl (especially halogen). Examples of such groups as used for the substituent R¹ are difluoromethoxy-phenyl (e.g. 4-difluoromethoxy-phenyl, 3-difluoromethoxy-phenyl), fluoro-difluoromethoxy-phenyl (e.g. 3-fluoro-4-difluoromethoxy-phenyl, 2-fluoro-4-difluoromethoxy-phenyl, 2-fluoro-3-difluoromethoxy-phenyl, 4-fluoro-3-difluoromethoxy-phenyl, 5-fluoro-3-difluoromethoxy-phenyl, 6-fluoro-3-difluoromethoxy-phenyl), and difluoro-difluoromethoxy-phenyl (e.g. 2,3-difluoro-4-difluoromethoxy-phenyl, 2,6-difluoro-4-difluoromethoxy-phenyl, 2,5-difluoro-4-difluoromethoxy-phenyl, 3,5-difluoro-4-difluoromethoxy-phenyl, 2,4-difluoro-3-difluoromethoxy-phenyl, 2,5-difluoro-3-difluoromethoxy-phenyl, 2,6-difluoro-3-difluoromethoxy-phenyl, 4,5-difluoro-3-difluoromethoxy-phenyl). In a sub-embodiment, preferred are difluoromethoxy-phenyl and fluoro-difluoromethoxy-phenyl. In another embodiment, preferably, the above groups are phenyl groups, which are mono-, di-, or tri-substituted, wherein one substituent is difluoromethoxy (especially in position 3 or 4; in a sub-embodiment in position 3; in another sub-embodiment in position 4), and the remaining substituents (if present) are fluorine. Preferred examples of such groups are 4-difluoromethoxy-phenyl, 3-difluoromethoxy-phenyl, 3-fluoro-4-difluoromethoxy-phenyl, 2-fluoro-3-difluoromethoxy-phenyl, 4-fluoro-3-difluoromethoxy-phenyl, 5-fluoro-3-difluoromethoxy-phenyl, 2,3-difluoro-4-difluoromethoxy-phenyl, and 3,5-difluoro-4-difluoromethoxy-phenyl. In a sub-embodiment, preferred examples of such groups are 4-difluoromethoxy-phenyl, 3-difluoromethoxy-phenyl, and 3-fluoro-4-difluoromethoxy-phenyl.

Any reference to a compound of formula (I) and/or (II) is to be understood as referring also to the salts (and especially the pharmaceutically acceptable salts) of such compounds, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

The compounds of formula (I) and/or (II) may contain two or more stereogenic or asymmetric centers, such as two or more asymmetric carbon atoms. The compounds of formula (I) and/or (II) may thus be present as mixtures of stereoisomers or preferably as pure stereoisomers. Mixtures of stereoisomers may be separated in a manner known to a person skilled in the art.

The present invention also includes all suitable isotopic variations of a compound of formula (I). Such isotopically labelled compound is identical to the compound of formula (I) wherein one or more atoms have been replaced by an atom having the same atomic number but an atomic mass different from the atomic mass usually found in nature. Examples of isotopes that can be incorporated into compounds of formula (I) include isotopes of hydrogen, carbon, nitrogen, oxygen, fluorine, iodine, and chlorine; such as ²H, ³H, ¹¹C, ¹⁴C, ¹⁵N, ¹⁷O, ¹⁸O, ¹⁸F, ³⁵S, ³⁶Cl, ¹²³I, and ¹²⁵I. Isotopically labelled compounds of formula (I) and salts thereof are within the scope of the present invention. Such isotopically labelled compounds are useful in drug distribution assays (e.g. ³H, ¹⁴C); positron emission tomography PET (¹¹C, ¹⁸F); or single photon emission computerized tomography SPECT (¹²⁵I). Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula (I) are not isotopically labelled, or labelled with one or more deuterium atoms. In a sub-embodiment, the compounds of formula (I) are not isotopically labelled. Isotopically labelled compounds of formula (I) may be prepared in analogy to the methods described hereinafter, but using the appropriate isotopic variation of suitable reagents or starting materials.

Further embodiments of the invention are presented hereafter:

2) A further embodiment of the invention relates to compounds of formula (I) according to embodiment 1), wherein the absolute configuration is [(R)-2′; (S)-8] or [(R)-2′; (R)-8].

3) A further embodiment of the invention relates to compounds of formula (I) according to embodiment 1) or 2) which are also compounds of formula (II), wherein the absolute configuration is [(R)-2′; (S)-8]:

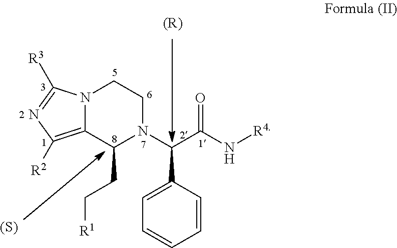

4) A further embodiment relates to compounds of formula (I) according to any one of embodiments 1) to 3), wherein R⁴ represents methyl.

5) A further embodiment relates to compounds of formula (I) according to any one of embodiments 1) to 4), wherein R¹, R², and R³ represent one of the following combinations:
R³ represents cyclopropyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl.

6) A further embodiment relates to compounds of formula (I) according to any one of embodiments 1) to 4), wherein R¹, R², and R³ represent one of the following combinations:
R³ represents cyclopropyl;
R² represents trifluoromethyl, or (C₁₋₄)alkyl (especially trifluoromethyl); and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents trifluoromethyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents trifluoromethyl;
R² represents (C₁₋₄)alkyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl.

7) A further embodiment relates to compounds of formula (I) according to any one of embodiments 1) to 4), wherein R¹, R², and R³ represent one of the following combinations:
R³ represents cyclopropyl;
R² represents halogen, trifluoromethyl, (C₁₋₄)alkyl, or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
wherein the above combination may be further characterized by the following sub-embodiments: in one sub-embodiment R² represents halogen, trifluoromethyl, or (C₁₋₄)alkyl (especially (C₁₋₄)alkyl or trifluoromethyl); in another sub-embodiment R² represents halogen; in another sub-embodiment R² represents trifluoromethyl; in another sub-embodiment R² represents (C₁₋₄)alkyl; and in yet another sub-embodiment R² represents vinyl;
or
R³ represents (C₃₋₆)cycloalkyl-(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl.

8) A further embodiment relates to compounds of formula (I) according to any one of embodiments 1) to 7), wherein R³ represents cyclopropyl.

9) A further embodiment relates to compounds of formula (I) according to any one of embodiments 1) to 4) or 7), wherein R³ represents (C₃₋₆)cycloalkyl-(C₁₋₄)alkyl; another embodiment relates to said compounds according to any one of embodiments 1) to 4) or 7), wherein R³ represents a group different from (C₃₋₆)cycloalkyl-(C₁₋₄)alkyl.

10) A further embodiment relates to compounds of formula (I) according to any one of embodiments 1) to 5) or 7), wherein
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl.

11) A further embodiment relates to compounds of formula (I) according to any one of embodiments 1) to 4), wherein R¹, R², and R³ represent one of the following combinations:
R³ represents —S—(C₁₋₄)alkyl;
R² represents halogen, trifluoromethyl or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
wherein the above combination may be further characterized by the following sub-embodiments: in one sub-embodiment R² represents halogen, or trifluoromethyl; in another sub-embodiment R² represents halogen; in another sub-embodiment R² represents trifluoromethyl; and in yet another sub-embodiment R² represents vinyl;
or
R³ represents (C₁₋₄)alkyl;
R² represents —S{O}ₙ—(C₁₋₄)alkyl, wherein n represents the integer 0 or 2; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl.

12) A further embodiment relates to compounds of formula (I) according to any one of embodiments 1) to 6) or 11), wherein R³ represents —S—(C₁₋₄)alkyl.

13) A further embodiment relates to compounds of formula (I) according to any one of embodiments 1) to 4) or 11), wherein R² represents —S{O}ₙ—(C₁₋₄)alkyl, wherein n represents the integer 0 or 2.

14) A further embodiment relates to compounds of formula (I) according to any one of embodiments 1) to 4), wherein R³ represents (C₁₋₄)alkoxy.

15) A further embodiment relates to compounds of formula (I) according to any one of embodiments 1) to 4), or 6), wherein R³ represents trifluoromethyl.

16) A further embodiment relates to compounds of formula (I) according to any one of embodiments 1) to 5) or 7) to 12), wherein R² represents halogen.

17) A further embodiment relates to compounds of formula (I) according to any one of embodiments 1) to 4), 6) to 8), 11), or 12), wherein R² represents trifluoromethyl.

18) A further embodiment relates to compounds of formula (I) according to any one of embodiments 1) to 4), or 6) to 8), wherein R² represents (C₁₋₄)alkyl.

19) A further embodiment relates to compounds of formula (I) according to any one of embodiments 1) to 4), 11) or 13), wherein n represents the integer 0.

20) A further embodiment relates to compounds of formula (I) according to any one of embodiments 1) to 4), 11) or 13), wherein n represents the integer 2.

21) A further embodiment relates to compounds of formula (I) according to any one of embodiments 1) to 9) or 11) to 20), wherein, if not explicitly stated otherwise, R¹ represents a phenyl group, which group is mono-, di-, or tri-substituted, wherein the substituents are independently selected from the group consisting of (C₁₋₄)alkyl, (C₁₋₄)alkoxy, halogen, difluoromethoxy, trifluoromethoxy and trifluoromethyl (especially the substituents are independently selected from the group consisting of halogen, difluoromethoxy, trifluoromethoxy and trifluoromethyl).

22) A further embodiment relates to compounds of formula (I) according to any one of embodiments 1) to 20), wherein R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, difluoromethoxy, trifluoromethoxy and trifluoromethyl (especially halogen).

23) A further embodiment relates to compounds of formula (I) according to any one of embodiments 1) to 9) or 11) to 20), wherein, if not explicitly stated otherwise, R¹ represents a phenyl group, which is mono-, di-, or tri-substituted, wherein one substituent is difluoromethoxy, trifluoromethoxy or trifluoromethyl (especially in position 3 or 4; in a sub-embodiment in position 3; in another sub-embodiment in position 4), and the remaining substituents (if present) are fluorine.

24) A further embodiment relates to compounds of formula (I) according to any one of embodiments 1) to 20), wherein R¹ represents a phenyl group, which group is mono-, di-, or tri-substituted, wherein one substituent is difluoromethoxy (especially in position 3 or 4; in a sub-embodiment in position 3; in another sub-embodiment in position 4), and the remaining substituents (if present) are fluorine.

25) A further embodiment relates to compounds of formula (I) according to any one of embodiments 1) to 9) or 11) to 20), wherein, if not explicitly stated otherwise, R¹ represents a phenyl group, which is mono-, or di-substituted, wherein one substituent is difluoromethoxy, trifluoromethoxy or trifluoromethyl in position 3 or 4 (in a sub-embodiment in position 3; in another sub-embodiment in position 4), and the remaining substituent (if present) is fluorine.

26) A further embodiment relates to compounds of formula (I) according to any one of embodiments 1) to 20), wherein R¹ represents a phenyl group, which group is mono-, or di-substituted, wherein one substituent is difluoromethoxy in position 3 or 4 (in a sub-embodiment in position 3; in another sub-embodiment in position 4), and the remaining substituent (if present) is fluorine.

27) A further embodiment relates to compounds of formula (I) according to any one of embodiments 1) to 9) or 11) to 20), wherein, if not explicitly stated otherwise, R¹ represents a phenyl group, which is tri-substituted, wherein one substituent is difluoromethoxy, trifluoromethoxy or trifluoromethyl in position 3 or 4 (in a sub-embodiment in position 3; in another sub-embodiment in position 4), and the remaining substituents are fluorine.

28) A further embodiment relates to compounds of formula (I) according to any one of embodiments 1) to 20), wherein, R¹ represents a phenyl group, which is tri-substituted, wherein one substituent is difluoromethoxy in position 3 or 4 (in a sub-embodiment in position 3; in another sub-embodiment in position 4), and the remaining substituents are fluorine.

29) In another embodiment of the invention compounds of formula (I) according to embodiment 1) are selected from the group consisting of:
(R)-2′-{1-chloro-3-cyclopropyl-(S)-8-[2-(4-fluoro-3-trifluoromethyl-phenyl)-ethyl]-5,6-dihydro-8H-imidazo[1,5-a]pyrazin-7-yl}-N-methyl-2′-phenyl-acetamide;
(R)-2′-{1-chloro-(S)-8-[2-(4-chloro-3-fluoro-phenyl)-ethyl]-3-cyclopropyl-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(4-difluoromethoxy-phenyl)-ethyl]-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(3-fluoro-4-trifluoromethoxy-phenyl)-ethyl]-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(4-difluoromethoxy-3-fluoro-phenyl)-ethyl]-5,6-dihydro-8H-imidazo[1,5-a]pyrazin-7-yl}-N-methyl-2′-phenyl-acetamide;
(R)-2′-{1-chloro-3-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{1-chloro-(S)-8-[2-(2,3-difluoro-4-trifluoromethyl-phenyl)-ethyl]-3-methylsulfanyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-3-fluoro-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(3-difluoromethoxy-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-phenyl)-ethyl]-3-methyl-5,6-dihydro-8H-imidazo[1,5-a]pyrazin-7-yl}-N-methyl-2′-phenyl-acetamide;
N-methyl-(R)-2′-{1-methyl-3-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide;
N-methyl-(R)-2′-{3-methylsulfanyl-1-trifluoromethyl-(S)-8-[2-(4-trifluoromethyl-phenyl)-ethyl]-5,6-dihydro-8H-imidazo[1,5-a]pyrazin-7-yl}-Z-phenyl-acetamide;
(R)-2′-{3-cyclopropyl-(S)-8-[2-(3-fluoro-4-trifluoromethyl-phenyl)-ethyl]-1-trifluoromethyl-5,6-dihydro-8H-imidazo[1,5-a]pyrazin-7-yl}-N-methyl-2′-phenyl-acetamide;
(R)-2′-{3-cyclopropyl-1-ethyl-(S)-8-[2-(3-fluoro-4-trifluoromethyl-phenyl)-ethyl]-5,6-dihydro-8H-imidazo[1,5-a]pyrazin-7-yl}-N-methyl-Z-phenyl-acetamide;
(R)-2′-{3-cyclopropyl-(S)-8-[2-(3-fluoro-4-trifluoromethyl-phenyl)-ethyl]-1-vinyl-5,6-dihydro-8H-imidazo[1,5-a]pyrazin-7-yl}-N-methyl-Z-phenyl-acetamide;
N-methyl-(R)-2′-{3-methylsulfanyl-(S)-8-[2-(4-trifluoromethyl-phenyl)-ethyl]-1-vinyl-5,6-dihydro-8H-imidazo[1,5-a]pyrazin-7-yl}-2′-phenyl-acetamide;
(R)-2′-{3-ethyl-1-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{3-ethoxy-1-trifluoromethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide; and
(R)-2′-{3-ethyl-1-methanesulfonyl-(S)-8-[2-(4-trifluoromethyl-phenyl)-ethyl]-5,6-dihydro-8H-imidazo[1,5-a]pyrazin-7-yl}-N-methyl-2′-phenyl-acetamide.

30) In another embodiment of the invention, in addition to the compounds listed in embodiment 29), compounds of formula (I) according to embodiment 1) are selected from the group consisting of:
(R)-2′-{1-ethyl-(S)-8-[2-(3-fluoro-4-trifluoromethyl-phenyl)-ethyl]-3-trifluoromethyl-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{1-ethyl-3-trifluoromethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(3-trifluoromethyl-phenyl)-ethyl]-5,6-dihydro-8H-imidazo[1,5-a]pyrazin-7-yl}-N-methyl-2′-phenyl-acetamide; and
(R)-2′-{1-chloro-3-cyclopropyl-(S)-8-[2-(2-fluoro-4-trifluoromethyl-phenyl)-ethyl]-5,6-dihydro-8H-imidazo[1,5-a]pyrazin-7-yl}-N-methyl-2′-phenyl-acetamide.

The compounds of formula (I) and/or (II) and their pharmaceutically acceptable salts can be used as medicaments, e.g. in the form of pharmaceutical compositions for enteral or parenteral administration.

A further aspect of the invention is a pharmaceutical composition containing at least one compound according to formula (I) and/or (II), or a pharmaceutically acceptable salt thereof, and a pharmaceutically acceptable carrier material.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of Formula (I) and (II) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Where the plural form is used for compounds, salts, pharmaceutical compositions, diseases or the like, this is intended to mean also a single compound, salt, disease or the like.

In one embodiment, the invention relates to a method for the treatment and/or prevention of the diseases mentioned herein, said method comprising administering to a subject a pharmaceutically active amount of a compound of formula (I) and/or (II).

For avoidance of any doubt, if compounds are described as useful for the prevention or treatment of certain diseases, such compounds are likewise suitable for use in the preparation of a medicament for the prevention or treatment of said diseases.

The compounds of formula (I) and/or (II) may be used for the preparation of a medicament and are suitable for the treatment and/or prevention of the diseases related to the orexin system.

Such diseases related to the orexin system may be selected from the group consisting of selected from the group consisting of dysthymic disorders including major depression and cyclothymia, affective neurosis, all types of manic depressive disorders, delirium, psychotic disorders, schizophrenia, catatonic schizophrenia, delusional paranoia, adjustment disorders and all clusters of personality disorders; schizoaffective disorders; anxiety disorders including generalized anxiety, obsessive compulsive disorder, posttraumatic stress disorder, panic attacks, all types of phobic anxiety and avoidance; separation anxiety; all psychoactive substance use, abuse, seeking and reinstatement; all types of psychological or physical addictions, dissociative disorders including multiple personality syndromes and psychogenic amnesias; sexual and reproductive dysfunction; psychosexual dysfunction and addiction; tolerance to narcotics or withdrawal from narcotics; increased anaesthetic risk, anaesthetic responsiveness; hypothalamic-adrenal dysfunctions; disturbed biological and circadian rhythms; sleep disturbances associated with diseases such as neurological disorders including neuropathic pain and restless leg syndrome; sleep apnea; narcolepsy; chronic fatigue syndrome; insomnias related to psychiatric disorders; all types of idiopathic insomnias and parasomnias; sleep-wake schedule disorders including jet-lag; all dementias and cognitive dysfunctions in the healthy population and in psychiatric and neurological disorders; mental dysfunctions of aging; all types of amnesia; severe mental retardation; dyskinesias and muscular diseases; muscle spasticity, tremors, movement disorders; spontaneous and medication-induced dyskinesias; neurodegenerative disorders including Huntington's, Creutzfeld-Jacob's, Alzheimer's diseases and Tourette syndrome; Amyotrophic lateral sclerosis; Parkinson's disease; Cushing's syndrome; traumatic lesions; spinal cord trauma; head trauma; perinatal hypoxia; hearing loss; tinnitus; demyelinating diseases; spinal and cranial nerve diseases; ocular damage; retinopathy; epilepsy; seizure disorders; absence seizures, complex partial and generalized seizures; Lennox-Gastaut syndrome; migraine and headache; pain disorders; anaesthesia and analgesia; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; dental pain; pain related to infection e.g. by HIV; post-chemotherapy pain; post-stroke pain; post-operative pain; neuralgia; osteoarthritis; conditions associated with visceral pain such as irritable bowel syndrome; eating disorders; diabetes; toxic and dysmetabolic disorders including cerebral anoxia, diabetic neuropathies and alcoholism; appetite, taste, eating, or drinking disorders; somatoform disorders including hypochondriasis; vomiting/nausea; emesis; gastric dyskinesia; gastric ulcers; Kallman's syndrome (anosmia); impaired glucose tolerance; intestinal motility dyskinesias; hypothalamic diseases; hypophysis diseases; hyperthermia syndromes, pyrexia, febrile seizures, idiopathic growth deficiency; dwarfism; gigantism; acromegaly; basophil adenoma; prolactinoma; hyperprolactinemia; brain tumors, adenomas; benign prostatic hypertrophy, prostate cancer; endometrial, breast, colon cancer; all types of testicular dysfunctions, fertility control; reproductive hormone abnormalities; hot flashes; hypothalamic hypogonadism, functional or psychogenic amenorrhea; urinary bladder incontinence asthma; allergies; all types of dermatitis, acne and cysts, sebaceous gland dysfunctions; cardiovascular disorders; heart and lung diseases, acute and congestive heart failure; hypotension; hypertension; dyslipidemias, hyperlipidemias, insulin resistance; urinary retention; osteoporosis; angina pectoris; myocardial infarction; arrhythmias, coronary diseases, left ventricular hypertrophy; ischemic or haemorrhagic stroke; all types of cerebrovascular disorders including subarachnoid haemorrhage, ischemic and hemorrhagic stroke and vascular dementia; chronic renal failure and other renal diseases; gout; kidney cancer; urinary incontinence; and other diseases related to general orexin system dysfunctions.

In particular, such diseases related to the orexin system may be selected from the group consisting of all types of sleep disorders, of stress-related syndromes, of addictions (especially psychoactive substance use, abuse, seeking and reinstatement), of cognitive dysfunctions in the healthy population and in psychiatric and neurologic disorders, of eating or drinking disorders.

Eating disorders may be defined as comprising metabolic dysfunction; dysregulated appetite control; compulsive obesities; emeto-bulimia or anorexia nervosa. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance. Drinking disorders include polydipsias in psychiatric disorders and all other types of excessive fluid intake. Sleep disorders include all types of parasomnias, insomnias, narcolepsy and other disorders of excessive sleepiness, sleep-related dystonias; restless leg syndrome; sleep apneas; jet-lag syndrome; shift-work syndrome, delayed or advanced sleep phase syndrome or insomnias related to psychiatric disorders. Insomnias are defined as comprising sleep disorders associated with aging; intermittent treatment of chronic insomnia; situational transient insomnia (new environment, noise) or short-term insomnia due to stress; grief; pain or illness. Insomnia also include stress-related syndromes including post-traumatic stress disorders as well as other types and subtypes of anxiety disorders such as generalized anxiety, obsessive compulsive disorder, panic attacks and all types of phobic anxiety and avoidance. Addictions may be defined as addiction to one or more rewarding stimuli, notably to one rewarding stimulus. Such rewarding stimuli may be of either natural or synthetic origin. Psychoactive substance use, abuse, seeking and reinstatement are defined as all types of psychological or physical addictions and their related tolerance and dependence components. Cognitive dysfunctions include deficits in all types of attention, learning and memory functions occurring transiently or chronically in the normal, healthy, young, adult or aging population, and also occurring transiently or chronically in psychiatric, neurologic, cardiovascular and immune disorders.

In a sub-embodiment, such diseases related to the orexin system may be selected from the group consisting of sleep disorders that comprises all types of insomnias, narcolepsy and other disorders of excessive sleepiness, sleep-related dystonias, restless leg syndrome, sleep apneas, jet-lag syndrome, shift-work syndrome, delayed or advanced sleep phase syndrome or insomnias related to psychiatric disorders.

In another sub-embodiment, such diseases related to the orexin system may be selected from the group consisting of cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders.

In another sub-embodiment, such diseases related to the orexin system may be selected from the group consisting of eating disorders that comprise metabolic dysfunction; dysregulated appetite control; compulsive obesities; emeto-bulimia or anorexia nervosa.

In another sub-embodiment, such diseases related to the orexin system may be selected from the group consisting of all types of addictions (especially psychoactive substance use, abuse, seeking and reinstatement) that comprise all types of psychological or physical addictions and their related tolerance and dependence components.

Compounds of formula (I) of the present invention can be prepared according to the general sequence of reactions outlined in the schemes below

A further aspect of the invention is a process for the preparation of compounds of formula (I) and/or (II). Compounds of formula (I) and/or (II) may be prepared according to several synthetic routes described below (schemes 1 to 16), wherein R¹, R², R³, and R⁴ are as defined for formula (I). All chemical transformations can be performed according to well-known standard methodologies as described in the literature or as described in the procedures below. Starting materials are commercially available or prepared according to procedures known in the literature or as illustrated herein. The order of carrying out the mentioned synthetic routes may be varied to facilitate the reaction or to avoid side-products. The compounds obtained may also be converted into pharmaceutically acceptable salts thereof in a manner known per se.

An overview of the general synthetic route is presented in scheme 1. Tri-substituted-imidazole derivatives represent key intermediates in this synthesis, and therefore their regioselective preparation was envisaged. Thus, the issue of tautomerism associated with imidazoles (and leading to isomeric mixtures) may be circumvented in this approach through the use of pseudosymmetric 4,5-diiodoimidazole derivatives. Diiodination (e.g. using I₂/Na₂CO₃/dioxane/H₂O) of 2-substituted imidazoles A (from commercial sources or specifically synthesized, see below) gives the corresponding 4,5-diiodoimidazoles B. Deprotonation of pseudosymmetric B (NaH/DMF), and subsequent N-alkylation with Br(CH₂)₂NHBoc furnishes the product C. A pivotal step of this synthetic route is the preparation of the corresponding 4-iodoimidazoles D by using a regioselective exchange of the 5-iodo moiety for MgBr (EtMgBr/THF/−40° C.) followed by trapping of the carbanion with water, leading to 4-iodoimidazole derivatives D. Boc-deprotection of D provides the corresponding primary amines E which may be reacted with aldehydes R¹—CH₂—CH₂—CHO e.g. in a microwave-assisted Pictet-Spengler-like reaction. Subsequent Boc-protection and purification affords the 5,6,7,8-tetrahydro-imidazo[1,5-a]pyrazine derivatives F. The versatility of the iodo-substituent allows the access to a variety of derivatives G (see schemes 2a/2b). Boc-deprotection of G, and N-alkylation with electrophiles H (see schemes 15, 16) furnishes compounds of formula (I).

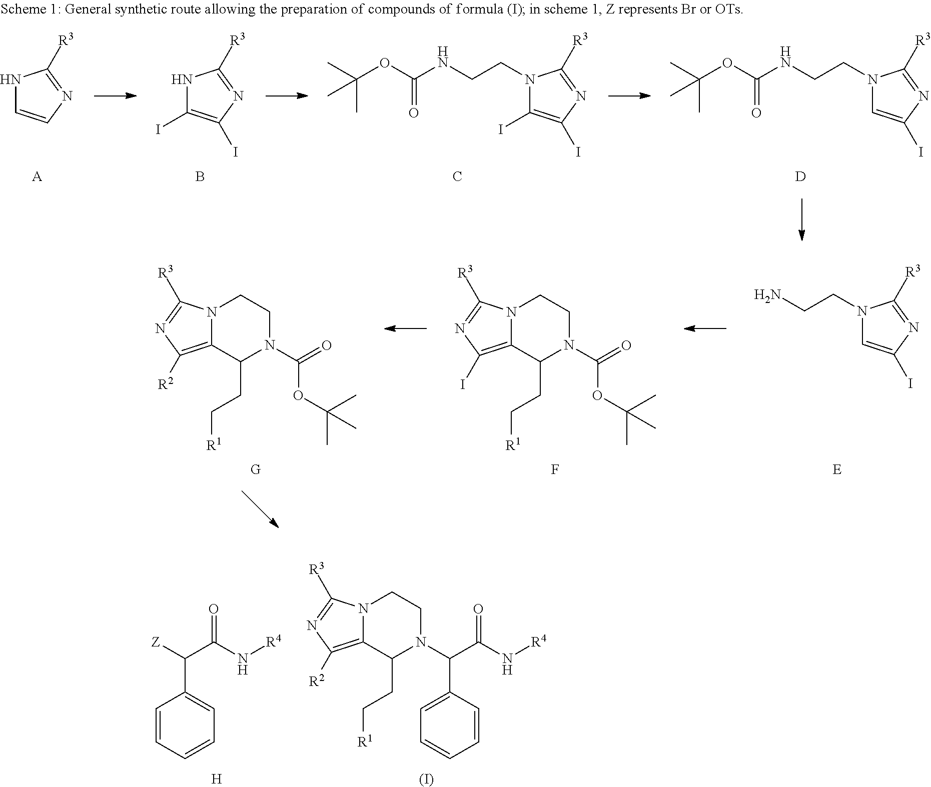

The versatility of the iodo-substituent in the 5,6,7,8-tetrahydro-imidazo[1,5-a]pyrazines F allows the preparation of a variety of derivatives (schemes 2a/2b). Thus, treatment of F with n-butyllithium followed by trapping of the resulting carbanion with hexachloroethane affords the corresponding chloro-derivative. Alternatively, introduction of the chloro- or bromo-substituent can be achieved by application of the sequence depicted in scheme 2a. Thus, preliminary hydrogenolytic cleavage of the iodo-substituent in F (H₂/Pd(C)/K₂CO₃/MeOH) followed by chlorination (NCS/MeCN) or bromination (NBS/MeCN) affords the halogenated derivative with higher overall yields. In another approach, the iodo-substituent allows the insertion of the trifluoromethyl moiety via (trifluoromethyl)copper-mediated trifluoromethylation (FSO₂CF₂CO₂Me/CuI/HMPA/DMF). Thioalkyl residues may also be introduced (RSNa/CuCl/NMP) in the iodo-derivatives F, and the related sulfones may be obtained after a subsequent S-oxidation (MCPBA/DCM).

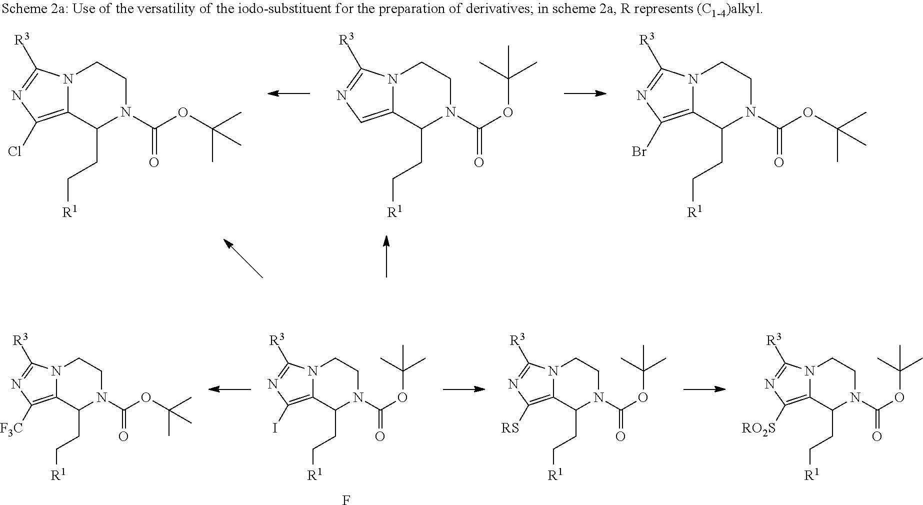

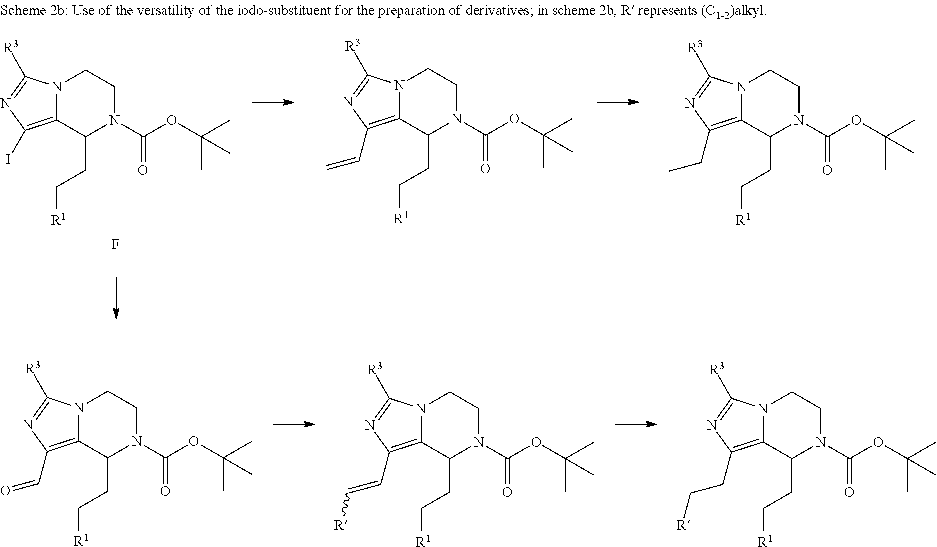

According to literature, iodo-imidazoles are known to be good substrates for Stille cross-coupling reactions, and the 5,6,7,8-tetrahydro-imidazo[1,5-a]pyrazines F react with n-tributyl(vinyl)tin (scheme 2b). In a next step, the introduced vinyl moiety may be hydrogenated affording the corresponding ethyl-substituted derivatives. The carbanion generated after iodine/metal exchange (EtMgBr) can be trapped with DMF, and the thus introduced formyl-moiety may be further manipulated (e.g. Wittig-type olefination) in order to prepare additional derivatives (scheme 2b). These introduced olefins may be hydrogenated (H₂/Pd(C)/MeOH) giving the corresponding saturated residues.

Compounds of formula (I), wherein R³ represents cyclopropyl, may be prepared according to scheme 3. Treatment of a mixture of aminoacetaldehyde dimethylacetal and cyclopropyl cyanide with cuprous chloride (CuCl) affords the corresponding amidine derivative. Cyclization of this intermediate to the desired 2-cyclopropyl-1H-imidazole can then realised in one pot by addition of HCl in MeOH. The resulting crude may be directly iodinated (I₂/Na₂CO₃/dioxane/H₂O) allowing at this stage the isolation of 2-cyclopropyl-4,5-diiodo-1H-imidazole (scheme 3). Remaining steps affording compounds of formula (I) are as previously described in the general synthesis depicted in schemes 1 and 2a/2b.

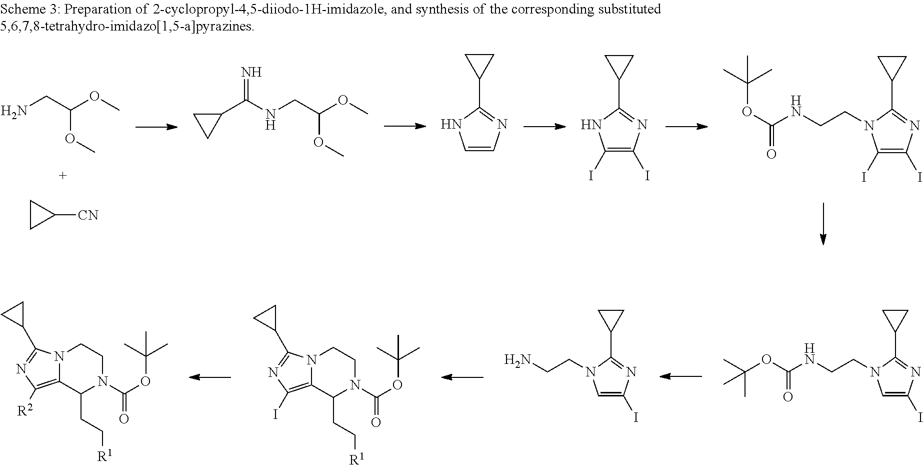

The preparation of compounds of formula (I), wherein R³ represents (C₃₋₆)cycloalkyl-(C₁₋₄)alkyl, is exemplified in scheme 4. 2-Cyclopropylmethyl-1H-imidazole may be prepared in analogy to the previously described CuCl-mediated reaction using cyclopropyl-acetonitrile and aminoacetaldehyde dimethylacetal (scheme 4). Remaining steps allowing access to compounds of formula (I) are as previously described.

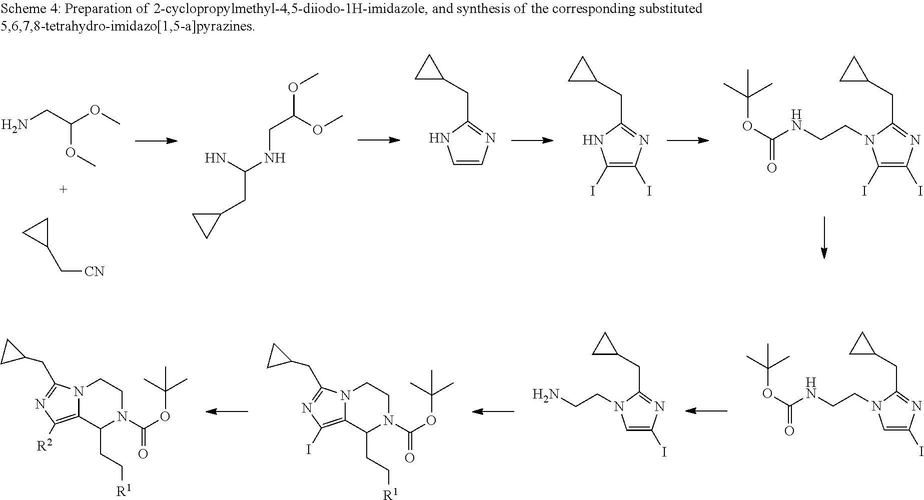

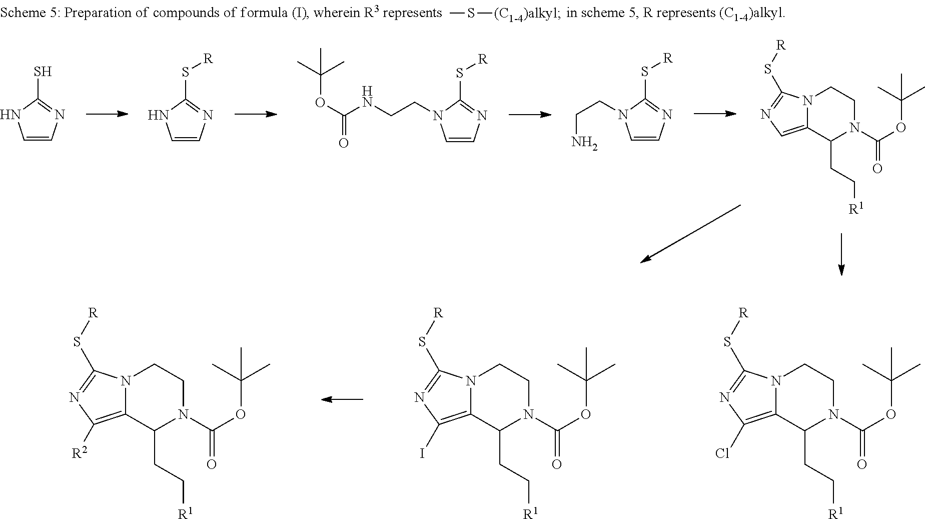

Compounds of formula (I), wherein R³ represents —S—(C₁₋₄)alkyl, may be obtained according to scheme 5. A selective S-alkylation of 1H-imidazole-2-thiol (alkyl halide/K₂CO₃/acetone) followed by N-alkylation with Br(CH₂)₂NHBoc, and Boc-deprotection affords the corresponding primary amines. These amines can then be reacted, e.g. in a microwave-assisted Pictet-Spengler like reaction, with aldehydes R¹—CH₂—CH₂—CHO. Subsequent Boc-protection, and purification affords the expected 5,6,7,8-tetrahydro-imidazo[1,5-a]pyrazine derivatives. At this stage, chlorination (NCS/MeCN) or iodination (N-iodosuccinimide/MeCN) provides the corresponding chloro- or iodo-derivatives. The versatility of the iodo-substituent allows the introduction of the R² substituents (scheme 5) as described above.

With these compounds in hand, the corresponding sulfones may be prepared through a subsequent S-oxidation (MCPBA/DCM) (scheme 6).

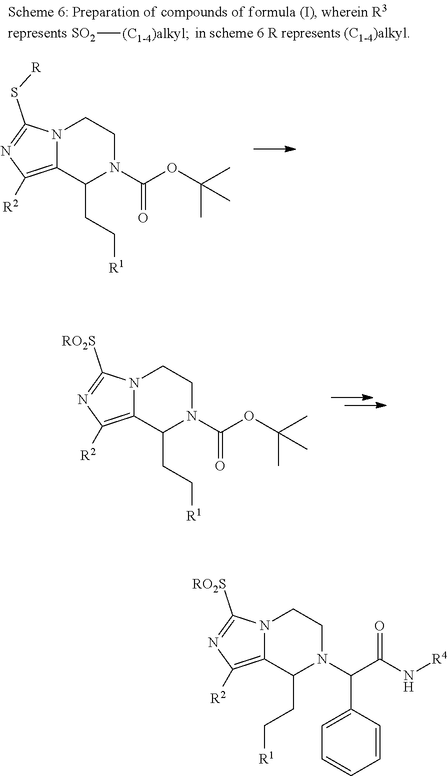

Through the placement of an appropriate electron-withdrawing substituent on the imidazole ring, these sulfonyl substituents (introduced according to scheme 6) can act as leaving groups allowing a convenient introduction of additional R³ substituents via ipso nucleophilic substitution. The application of this methodology is described in scheme 7 for the preparation of compounds of formula (I) wherein R³ represents (C₁₋₄)alkoxy. First, the trifluoromethyl moiety is introduced (FSO₂CF₂CO₂Me/CuI/HMPA/DMF). In a next step, the thioalkyl moiety may be oxidised to the corresponding sulfones (MCPBA/DCM) which can be reacted with anionic nucleophiles like alkoxides. Thus, treatment of these electron-deficient imidazole derivatives with various alkoxides (RONa/ROH/heating) affords the target alkoxy-derivatives (scheme 7).

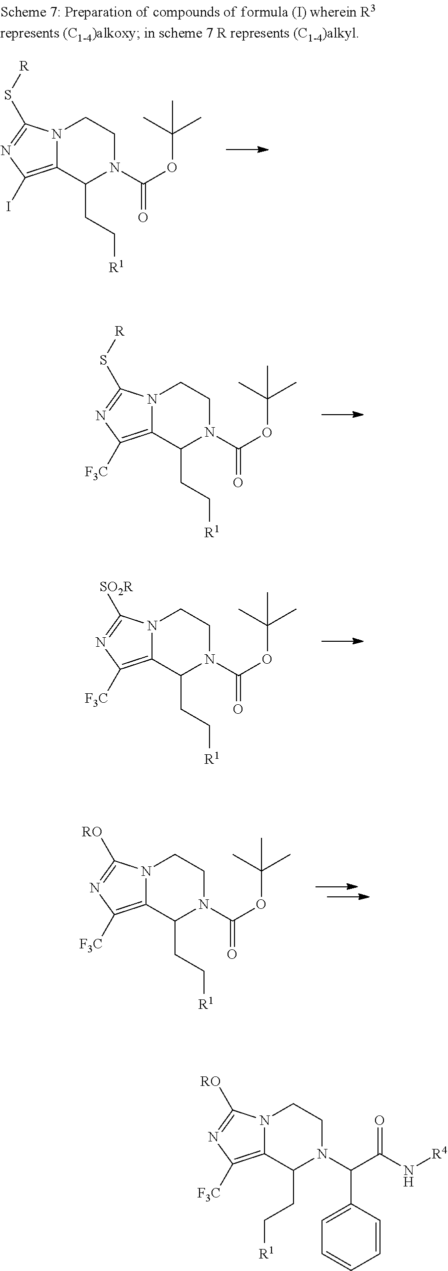

Scheme 8 describes the synthetic route to prepare compounds of formula (I) wherein R³ represents trifluoromethyl. The pivotal introduction of the trifluoromethyl moiety may be accomplished by fluoride ion induced cross-coupling reaction of a corresponding organic halide (bromide or iodide) with trifluoromethyltrialkylsilanes in the presence of copper(I) salts. The preparation of the appropriate bromo-derivatives starts with the iodination of imidazole (I₂/Na₂CO₃/dioxane/H₂O) affording 2,4,5-triiodo-1H-imidazole which may then be N-alkylated (NaH/BrCH₂CH₂NHBoc).

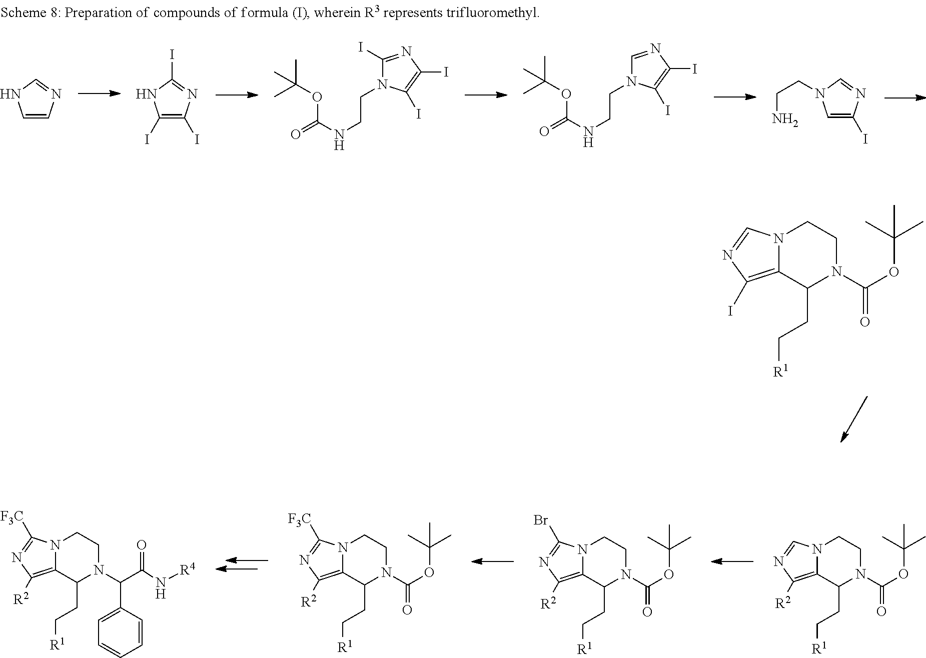

A subsequent regioselective one-pot removal of two iodo-substituents with EtMgBr (first on position-2, and secondly on position-5) furnishes the 4-iodoimidazole derivative which is Boc-deprotected (HCl in dioxane). The obtained primary amine may then be reacted with aldehydes R¹—CH₂—CH₂—CHO, e.g. in a microwave-assisted Pictet-Spengler like reaction. Subsequent Boc-protection affords the target 5,6,7,8-tetrahydro-imidazo[1,5-a]pyrazine derivatives. At this stage, the versatility of the iodo-substituent allows the introduction of the R² substituents (scheme 8). NBS-mediated bromination affords the 3-bromo-derivative which may be used in the pivotal trifluoromethylation reaction (CF₃SiMe₃/KF/CuI/DMF/NMP) as shown in scheme 8.

Closely related compounds wherein R² represents methyl may be prepared starting with commercially available 4(5)-methylimidazole according to scheme 9.

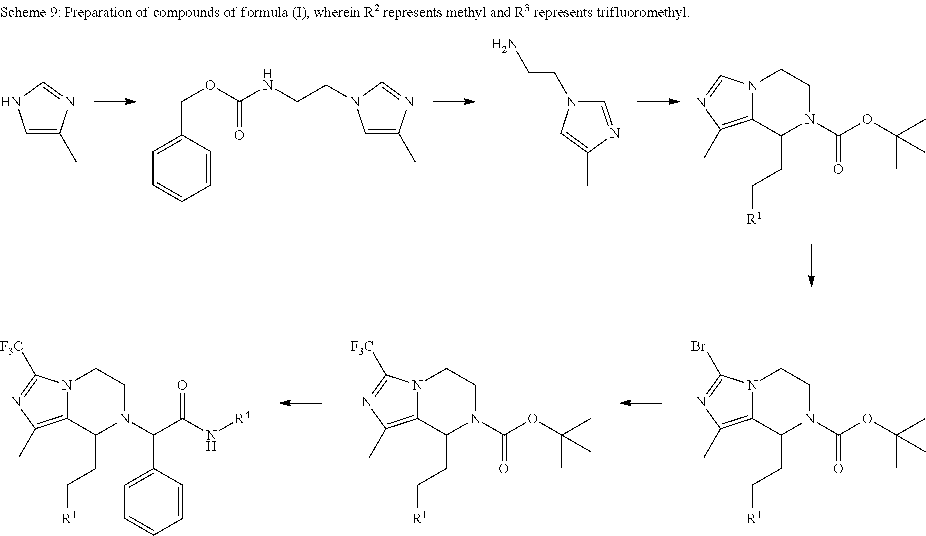

Aldehydes R¹—CH₂—CH₂—CHO are pivotal reagents for the preparation of compounds of formula (I), and several synthetic methods allow their preparation.

Thus, aldehydes R¹—CH₂—CH₂—CHO are readily prepared by reduction of the corresponding hydrocinnamic acids (BH₃.THF) and subsequent oxidation of the obtained primary alcohols with PCC (scheme 10). Preliminary hydrogenation of commercially available cinnamic acids allows access to hydrocinnamic acid precursors.

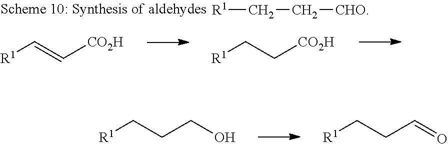

An alternative synthesis of diversely substituted 3-phenyl-propanol derivatives is the reduction of corresponding propionic acid methyl esters (scheme 11).

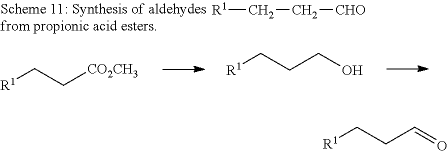

In case neither the appropriately substituted cinnamic acids nor hydrocinnamic acids are commercially available, additional synthetic routes allow their preparation. Thus, one synthetic pathway may be based on a Knoevenagel condensation as depicted in scheme 12. Knoevenagel condensation between aryl aldehydes R¹CHO and malonic acid (pyridine/piperidine/heating) gives the corresponding cinnamic acid derivatives. Subsequent catalytic hydrogenation under standard conditions (1 atm H₂/10% Pd(C)/MeOH/rt) affords the corresponding hydrocinnamic acids which may finally be converted to the corresponding aldehydes R¹—CH₂—CH₂—CHO according to the previously described reduction/oxidation sequence (scheme 12).

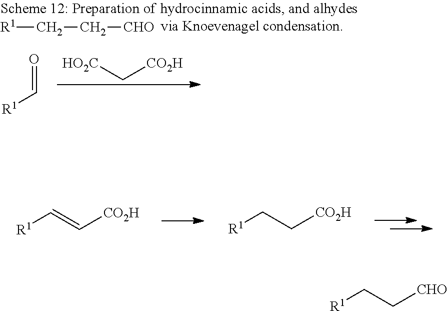

An alternative preparation of hydrocinnamic acids may be based on a Heck reaction between aryl halides and n-butyl acrylate (with Pd(OAc)₂/DABCO as catalytic system; scheme 13). Palladium-catalyzed hydrogenation, and subsequent saponification affords the corresponding hydrocinnamic acids which can again be converted to aldehydes R¹—CH₂—CH₂—CHO by the previously described reduction/oxidation sequence (scheme 13). Commercially unavailable aryl halides may be prepared via Sandmeyer reaction from the corresponding appropriately substituted aniline derivatives R¹NH₂.

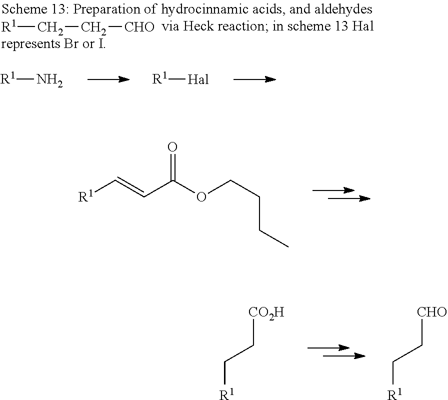

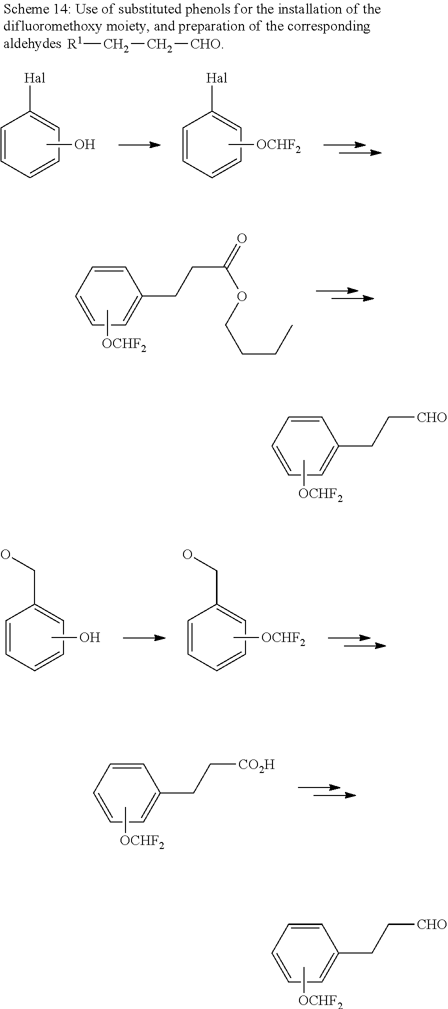

Aldehydes R¹—CH₂—CH₂—CHO containing the difluoromethoxy moiety may be prepared from commercially available precursors containing this residue according to previously presented synthetic routes. Alternatively, the difluoromethoxy group can be installed in aldehydes R¹—CH₂—CH₂—CHO by heating appropriate phenol derivatives with sodium chlorodifluoroacetate and K₂CO₃ in aq. DMF (scheme 14).

Regarding the final N-alkylation of substituted 5,6,7,8-tetrahydro-imidazo[1,5-a]pyrazines, in an alternative route (scheme 15) the secondary amine may be N-alkylated with ester derivatives (instead of amide derivative II) affording an intermediate ester which can either be directly transformed into target compounds (by reaction with amine R⁴—NH₂) or which can be first hydrolyzed to the corresponding carboxylic acid followed by coupling with amine R⁴—NH₂.

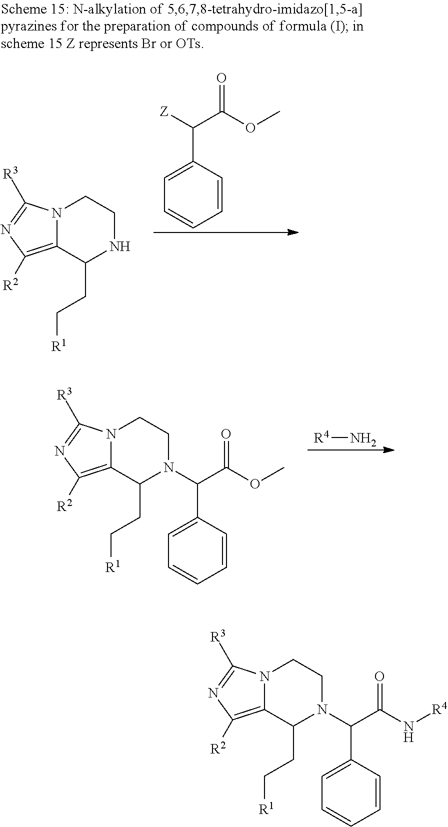

The synthesis of enantiomerically pure toluene-4-sulfonic acid (S)-methylcarbamoyl-phenyl-methyl ester is shown in scheme 16. Treatment of methyl (S)-(+)-mandelate with an alcoholic amine solution gives the corresponding amide which can be reacted with TsCl to toluene-4-sulfonic acid (S)-methylcarbamoyl-phenyl-methyl ester.

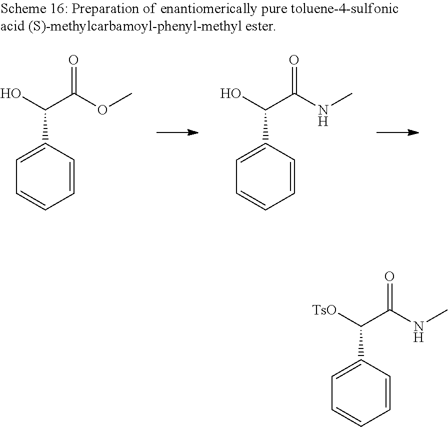

Whenever the compounds of formula (I) are obtained in the form of mixtures of enantiomers, the enantiomers can be separated using methods known to one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as TEA, diethylamine) and eluent B (hexane), at a flow rate of 0.8 to 150 mL/min.

### EXPERIMENTAL PART

### Abbreviations (As Used Herein and in the Description Above)

AcOEt ethyl acetate
AcOH acetic acid
anh. anhydrous
aq. aqueous
BH₃.THF borane-tetrahydrofuran complex
Boc tert-butoxycarbonyl
Boc₂O di-tert-butyl dicarbonate
Br(CH₂)₂NHBoc (2-Bromo-ethyl)-carbamic acid tert-butyl ester
n-BuLi n-butyllithium
CF₃SiMe₃ (trifluoromethyl)trimethylsilane
DABCO 1,4-diazabicyclo[2.2.2]octane
DCM dichloromethane

### DIPEA N,N-diisopropylethylamine

### DMF N,N-dimethylformamide

DMSO dimethyl sulfoxide

### ELSD Evaporative Light-Scattering Detection

eq. equivalent
Et ethyl
EtMgBr ethylmagnesium bromide
Et₂O diethyl ether
EtOH ethanol
FC flash chromatography on silica gel
FLIPR Fluorescent imaging plate reader
FSO₂CF₂CO₂Me methyl 2,2-difluoro-2-(fluorosulfonyl)acetate
h hour(s)
HCl hydrogen chloride
HMPA hexamethylphosphoramide
¹H-NMR nuclear magnetic resonance of the proton

### HPLC High Performance Liquid Chromatography

### HV High Vacuum

### LC-MS Liquid Chromatography-Mass Spectroscopy

M mol/l
MCPBA 3-chloroperbenzoic acid
MeCN acetonitrile
MeOH methanol
MsCl methanesulfonyl chloride
min. minute(s)
Ms methanesulfonyl

### MS Mass Spectroscopy

### NBS N-bromosuccinimide

### NCS N-chlorosuccinimide

NH₄OH ammonium hydroxide
NMP 1-methyl-2-pyrrolidinone
PBS phosphate buffered saline
PCC pyridinium chlorochromate
Pd(C) palladium over activated charcoal
Pd(OAc)₂ palladium (II) acetate
Ph phenyl
rt room temperature
sat. saturated
TEA triethylamine
TFA trifluoroacetic acid
THF tetrahydrofuran

### TLC Thin Layer Chromatography

t_{R} retention time
Ts toluenesulfonyl
TsCl p-toluenesulfonyl chloride
UV ultra violet
Vis visible

### W Watt

### I. CHEMISTRY: GENERAL PROCEDURES AND EXAMPLES

The following examples illustrate the preparation of biologically active compounds of the invention but do not at all limit the scope thereof.

All temperatures are stated in ° C.

The commercially available starting materials were used as received without further purification. Unless otherwise specified, all reactions were carried out in oven-dried glassware under an atmosphere of nitrogen.

Compounds are purified by column chromatography on silica gel or by preparative HPLC.

¹H-NMR: 300 MHz Varian Oxford or 400 MHz Bruker Avance; chemical shifts are given in ppm relative to the solvent used; multiplicities: s=singlet, d=doublet, t=triplet, m=multiplet, b=broad, coupling constants are given in Hz;

Compounds described in the invention are characterized by LC-MS data (retention time t_{R} is given in min.; molecular weight obtained from the mass spectrum is given in g/mol).

### Liquid Chromatography-Mass Spectroscopy (LC-MS) for Characterization of Compounds

LC-MS with Acidic Conditions:

Apparatus: Agilent 1100 series with mass spectroscopy detection (MS: Finnigan single quadrupole).

Column: Zorbax SB-AQ (4.6×50 mm) from Agilent Technologies.

Conditions: MeCN [eluent A]; water+0.04% TFA [eluent B]; Gradient: 95% B=>5% B over 1.5 min. (flow: 4.5 ml/min.).

If not explicitly stated otherwise acidic conditions have been used for the characterization of compounds which are described in the following experimental part.

Detection: UV/Vis+MS.

LC-MS with Basic Conditions:

Column: Zorbax Extend-C18 (4.6×50 mm) from Agilent Technologies.

Conditions: MeCN [eluent A]; 13 mmol/l NH₃ in water [eluent B]; Gradient: 95% B=>5% B over 1.5 min. (flow: 4.5 ml/min.).

### Preparative HPLC for Purification of Compounds

Column: Waters Xbridge, 75×30 mm.

Conditions: MeCN [eluent A]; water+0.05% NH₄OH (25% aq.) [eluent B]; Gradient: 90% B=>0% B over 6.5 min. (flow: 75 ml/min.)

Detection: UV+ELSD.

### A. Synthesis of carboxylic acids R¹—CH₂—CH₂—CO₂H, alcohols R¹—CH₂—CH₂—CH₂—OH, and aldehydes R¹—CH₂—CH₂—CHO

A.1 Synthesis of carboxylic acids R¹—CH₂—CH₂—CO₂H
A.1.1 Synthesis of carboxylic acids R¹—CH₂—CH₂—CO₂H via Knoevenagel Condensation
A.1.1.1 Preparation of cinnamic acids R¹—CH═CH—CO₂H via Knoevenagel Condensation
3-(3-fluoro-4-trifluoromethyl-phenyl)-acrylic acid
[General Procedure for the Preparation of Cinnamic acids Via Knoevenagel Condensation (GP1)]

A suspension of 3-fluoro-4-trifluoromethyl-benzaldehyde (10.000 g; 52.05 mmol; 1.0 eq.), and malonic acid (10.291 g; 98.90 mmol; 1.9 eq.) in pyridine (40 ml) was heated to 50° C., under nitrogen. Piperidine (4.0 ml; 40.49 mmol; 0.77 eq.) was then added dropwise (over 5 min.), and the resulting suspension was heated to 75° C. for 3 h. The reaction mixture was cooled to 0° C., and poured into an ice-cooled solution of concentrated hydrochloric acid (12 mol/l; 64 ml) in water (800 ml). The precipitated colorless product was filtered off, and washed with water (3×200 ml). Subsequent drying under HV afforded 3-(3-fluoro-4-trifluoromethyl-phenyl)-acrylic acid as a colorless solid (9.510 g; 78%). LC-MS: t_{R}=0.90 min.; [M+H]⁺: no ionisation.

3-(4-chloro-3-fluoro-phenyl)-acrylic acid

According to the described general procedure (GP1), Knoevenagel condensation (75° C.; 4 h 30) between 4-chloro-3-fluoro-benzaldehyde (11.000 g; 69.37 mmol) and malonic acid (13.716 g; 131.81 mmol) gave 3-(4-chloro-3-fluoro-phenyl)-acrylic acid as a colorless solid (13.460 g; 97%). LC-MS: t_{R}=0.92 min.; [M+H]⁺: no ionisation.

3-(4-difluoromethoxy-phenyl)-acrylic acid

According to the described general procedure (GP1), Knoevenagel condensation (75° C.; 3 h) between 4-difluoromethoxy-benzaldehyde (15.000 g; 82.78 mmol) and malonic acid (16.368 g; 157.29 mmol) gave 3-(4-difluoromethoxy-phenyl)-acrylic acid as a colorless solid (14.820 g; 84%). LC-MS: t_{R}=0.91 min.; [M+H]⁺: no ionisation.

3-(3-difluoromethoxy-phenyl)-acrylic acid

According to the described general procedure (GP1), Knoevenagel condensation (75° C.; 3 h) between 3-difluoromethoxy-benzaldehyde (17.000 g; 93.82 mmol) and malonic acid (18.550 g; 178.26 mmol) gave 3-(3-difluoromethoxy-phenyl)-acrylic acid as a colorless solid (17.880 g; 89%). LC-MS: t_{R}=0.91 min.; [M+H]⁺: no ionisation.

3-(3-trifluoromethyl-phenyl)-acrylic acid

According to the described general procedure (GP1), Knoevenagel condensation (75° C.; 3 h 20) between 3-trifluoromethyl-benzaldehyde (13.260 g; 76.15 mmol) and malonic acid (15.056 g; 144.69 mmol) gave 3-(3-trifluoromethyl-phenyl)-acrylic acid as a colorless solid (14.210 g; 86%). LC-MS: t_{R}=0.88 min.; [M+H]⁺: no ionisation.

3-(2-fluoro-4-trifluoromethyl-phenyl)-acrylic acid

According to the described general procedure (GP1), Knoevenagel condensation (75° C.; 3 h 20) between 2-fluoro-4-trifluoromethyl-benzaldehyde (5.000 g; 26.02 mmol) and malonic acid (5.145 g; 49.45 mmol) gave 3-(2-fluoro-4-trifluoromethyl-phenyl)-acrylic acid as a colorless solid (5.030 g; 82%). LC-MS: t_{R}=0.89 min.; [M+H]⁺: no ionisation.

A.1.1.2 Hydrogenation of Cinnamic acids R¹—CH═CH—CO₂H to the Corresponding Hydrocinnamic acids R¹—CH₂—CH₂—CO₂H
3-(3-fluoro-4-trifluoromethyl-phenyl)-propionic acid
[First General Procedure for Hydrogenation of Cinnamic acid Derivatives (GP2)]

A mixture of 3-(3-fluoro-4-trifluoromethyl-phenyl)-acrylic acid (9.510 g; 40.61 mmol), and 10% palladium on activated charcoal (950 mg; 10% w/w) was placed under nitrogen atmosphere before MeOH (120 ml) was added. The resulting black suspension was placed under vacuum, then under hydrogen atmosphere (1 atm), and the resulting reaction mixture was vigorously stirred at rt for 3.5 h. Filtration over a pad of celite, and concentration to dryness under reduced pressure afforded 3-(3-fluoro-4-trifluoromethyl-phenyl)-propionic acid as a grey solid (9.420 g; 98%). LC-MS: t_{R}=0.89 min.; [M+H]⁺: no ionisation.

3-(4-fluoro-3-trifluoromethyl-phenyl)-propionic acid

According to the previously described general procedure (GP2), hydrogenation (1 atm; rt; 6 h) of 3-(4-fluoro-3-trifluoromethyl-phenyl)-acrylic acid (10.300 g; 43.98 mmol) afforded 3-(4-fluoro-3-trifluoromethyl-phenyl)-propionic acid as a pale yellow solid (10.240 g; 99%). LC-MS: t_{R}=0.95 min.; [M+H]⁺: no ionisation.

3-(4-difluoromethoxy-phenyl)-propionic acid

According to the previously described general procedure (GP2), hydrogenation (1 atm; rt; 3 h) of 3-(4-difluoromethoxy-phenyl)-acrylic acid (14.820 g; 69.19 mmol) afforded 3-(4-difluoromethoxy-phenyl)-propionic acid as a slightly yellow solid (14.910 g; 99%). LC-MS: t_{R}=0.90 min.; [M+H]⁺: no ionisation.

3-(3-difluoromethoxy-phenyl)-propionic acid

According to the previously described general procedure (GP2), hydrogenation (1 atm; rt; 14 h) of 3-(3-difluoromethoxy-phenyl)-acrylic acid (17.870 g; 83.44 mmol) afforded 3-(3-difluoromethoxy-phenyl)-propionic acid as a pale yellow oil (17.890 g; 99%). LC-MS: t_{R}=0.90 min.; [M+H]⁺: no ionisation.

3-(3-trifluoromethyl-phenyl)-propionic acid

According to the previously described general procedure (GP2), hydrogenation (1 atm; rt; 2 h 30) of 3-(3-trifluoromethyl-phenyl)-acrylic acid (14.210 g; 65.73 mmol) afforded 3-(3-trifluoromethyl-phenyl)-propionic acid as a grey oil (12.390 g; 86%). LC-MS: t_{R}=0.87 min.; [M+H]⁺: no ionisation.

3-(2-fluoro-4-trifluoromethyl-phenyl)-propionic acid

According to the previously described general procedure (GP2), hydrogenation (1 atm; rt; 4 h) of 3-(2-fluoro-4-trifluoromethyl-phenyl)-acrylic acid (5.937 g; 25.35 mmol) afforded 3-(2-fluoro-4-trifluoromethyl-phenyl)-propionic acid as a grey solid (4.590 g; 77%). LC-MS: t_{R}=0.88 min.; [M+H]⁺: no ionisation.

3-(4-chloro-3-fluoro-phenyl)-propionic acid
[Second General Procedure for Hydrogenation of Cinnamic acid Derivatives (GP2B) in the Presence of Substituents Sensitive to Hydrogenation]

A mixture of 3-(4-chloro-3-fluoro-phenyl)-acrylic acid (6.000 g; 29.91 mmol; 1.0 eq.), ZnBr₂ (1.346 g; 5.98 mmol; 0.2 eq.), and 10% Pd(C) (600 mg; 10% in mass) was placed under nitrogen atmosphere before MeOH (500 ml) was added. The resulting black suspension was placed under vacuum, and then under hydrogen atmosphere (1 atm). This operation was repeated three times. The resulting reaction mixture was vigorously stirred at rt for 14 h. Filtration over a pad of celite, concentration to dryness under reduced pressure, and subsequent drying under HV afforded 3-(4-chloro-3-fluoro-phenyl)-propionic acid as an off-white solid (6.010 g; 99%). LC-MS: t_{R}=0.91 min.; [M+H]⁺: no ionisation.

A.1.2 Synthesis of carboxylic acids R¹—CH₂—CH₂—CO₂H Via Heck Reaction
A.1.2.1 Preparation of aryl halides
4-bromo-1-difluoromethoxy-2-fluoro-benzene

A mixture of 4-bromo-2-fluoro-phenol (3.0 ml; 27.38 mmol), K₂CO₃ (4.541 g; 32.85 mmol), sodium chlorodifluoroacetate (8.348 g; 54.76 mmol) in DMF (90 ml), and water (12 ml) was degassed by bubbling nitrogen into the suspension for 5 min., and was then heated to 100° C., under nitrogen, for 2.5 h. The heterogeneous mixture was allowed to cool to rt, and 12N HCl (8 ml; 96 mmol), and water (12 ml) were successively added, and this mixture was stirred at rt for 1 h. The resulting mixture was cooled to 0° C., and aq. 1N NaOH (100 ml) was then added portionwise. Et₂O (250 ml), and water (200 ml) were then added, and the yellow organic layer was further washed with water (150 ml), dried over anh. MgSO₄, filtered, and concentrated to dryness under reduced pressure. The crude material was purified by FC (DCM/heptane=1/1) to give 4-bromo-1-difluoromethoxy-2-fluoro-benzene as a slightly yellow oil (4.910 g; 74%). LC-MS: t_{R}=1.04 min.; [M+H]⁺: no ionisation.

1-bromo-2,3-difluoro-4-trifluoromethyl-benzene

A solution of 2,3-difluoro-4-trifluoromethyl-phenylamine (19.200 g; 97.41 mmol) in anh. MeCN (120 ml) was treated with copper(II) bromide CuBr₂ (21.757 g; 97.41 mmol), and the green heterogeneous mixture was heated to 45° C. A solution of tert-butyl nitrite (12.75 ml; 107.15 mmol) in MeCN (20 ml) was then added dropwise (over 25 min.), and the resulting mixture was further stirred at 45° C. for 2.5 h. The resulting dark-green heterogeneous reaction mixture was allowed to cool to rt, and was directly purified by FC (DCM). After concentration to dryness under reduced pressure, 1-bromo-2,3-difluoro-4-trifluoromethyl-benzene was obtained as an orange oil (22.960 g; 90%). LC-MS: t_{R}=1.08 min.; [M+H]⁺: no ionisation.

A.1.2.2 Heck Reaction Between aryl halides and butyl acrylate
3-(4-difluoromethoxy-3-fluoro-phenyl)-acrylic acid butyl ester

### [General Procedure for Heck Reaction Between Aryl Halides and Butyl Acrylate (GP3)]

A solution of 4-bromo-1-difluoromethoxy-2-fluoro-benzene (4.910 g; 20.37 mmol; 1.0 eq.) in anh. DMF (130 ml) was treated successively with butyl acrylate (4.35 ml; 30.55 mmol; 1.5 eq.), DABCO (91 mg; 0.81 mmol; 0.04 eq.), K₂CO₃ (2.815 g; 20.37 mmol; 1.0 eq.), and palladium acetate Pd(OAc)₂ (91 mg; 0.40 mmol; 0.02 eq.). The resulting orange suspension was heated to 120° C., under nitrogen, for 12 h. The black reaction mixture was allowed to cool to rt, and was then treated with Et₂O (150 ml), water (150 ml), and brine (75 ml). The orange organic layer was further washed with water (100 ml), and brine (25 ml). The resulting organic layer was dried over anh. MgSO₄, filtered, and concentrated to dryness under reduced pressure. Purification by FC (DCM/heptane=1/1) afforded 3-(4-difluoromethoxy-3-fluoro-phenyl)-acrylic acid butyl ester as a slightly yellow oil which was further dried under HV (4.650 g; 79%). LC-MS: t_{R}=1.13 min.; [M+H]⁺: no ionisation.

3-(3-fluoro-4-trifluoromethoxy-phenyl)-acrylic acid butyl ester

According to the described general procedure (GP3), 4-bromo-2-fluoro-1-trifluoromethoxy-benzene (15.000 g; 57.91 mmol), and butyl acrylate (12.38 ml; 86.87 mmol) gave after Heck reaction (120° C.; 2 h), and purification by FC (DCM/heptane=1/1) 3-(3-fluoro-4-trifluoromethoxy-phenyl)-acrylic acid butyl ester as a pale yellow oil (17.360 g; 98%). LC-MS: t_{R}=1.18 min.; [M+H]⁺: no ionisation.

3-(2,3-difluoro-4-trifluoromethyl-phenyl)-acrylic acid butyl ester

According to the described general procedure (GP3), 1-bromo-2,3-difluoro-4-trifluoromethyl-benzene (22.960 g; 87.97 mmol), and butyl acrylate (18.8 ml; 131.96 mmol) gave after Heck reaction (120° C.; 12 h), and purification by FC (DCM/heptane=1/1) 3-(2,3-difluoro-4-trifluoromethyl-phenyl)-acrylic acid butyl ester as a yellow oil (13.390 g; 49%). LC-MS: t_{R}=1.18 min.; [M+H]⁺: no ionisation.

A.1.2.3 Hydrogenation of cinnamic esters to the Corresponding hydrocinnamic acid Derivatives
3-(3-fluoro-4-trifluoromethoxy-phenyl)-propionic acid butyl ester

### [General Procedure for Hydrogenation of Cinnamic Esters (GP4)]

A mixture of 3-(3-fluoro-4-trifluoromethoxy-phenyl)-acrylic acid butyl ester (17.360 g; 56.68 mmol), and 10% Pd(C) (1.736 g; 10% in mass) was placed under nitrogen atmosphere before MeOH (200 ml) was added. The resulting suspension was placed under vacuum, then under hydrogen atmosphere (1 atm), and this procedure was repeated three times. The reaction mixture was then vigorously stirred at rt, under hydrogen (1 atm), for 2 h. Filtration over a pad of celite, and concentration to dryness under reduced pressure afforded 3-(3-fluoro-4-trifluoromethoxy-phenyl)-propionic acid butyl ester as a pale yellow oil which was further dried under HV (16.930 g; 97%). LC-MS: t_{R}=1.15 min.; [M+H]⁺: no ionisation.

3-(4-difluoromethoxy-3-fluoro-phenyl)-propionic acid butyl ester

According to the described general procedure (GP4), hydrogenation (1 atm; rt; 3 h 20) of 3-(4-difluoromethoxy-3-fluoro-phenyl)-acrylic acid butyl ester (11.060 g; 38.36 mmol) gave 3-(4-difluoromethoxy-3-fluoro-phenyl)-propionic acid butyl ester as a yellow oil (10.930 g; 98%). LC-MS: t_{R}=1.11 min.; [M+H]⁺: no ionisation.

3-(2,3-difluoro-4-trifluoromethyl-phenyl)-propionic acid butyl ester

According to the described general procedure (GP4), hydrogenation (1 atm; rt; 19.5 h) of 3-(2,3-difluoro-4-trifluoromethyl-phenyl)-acrylic acid butyl ester (13.150 g; 42.66 mmol) gave 3-(2,3-difluoro-4-trifluoromethyl-phenyl)-propionic acid butyl ester as a yellow oil (13.080 g; 99%). LC-MS: t_{R}=1.15 min.; [M+H]⁺: no ionisation.

A.1.2.4 Preparation of carboxylic acids R¹—CH₂—CH₂—CO₂H Via Saponification of Corresponding Esters
3-(4-difluoromethoxy-3-fluoro-phenyl)-propionic acid
[General Procedure for the Preparation of hydrocinnamic acid Derivatives Via Saponification of the Corresponding Esters (GP5)]

A slightly yellow solution of 3-(4-difluoromethoxy-3-fluoro-phenyl)-propionic acid butyl ester (8.300 g; 28.59 mmol; 1.0 eq.) in MeOH (132 ml), and water (33 ml) was treated dropwise (over 5 min.) at rt with aq. 1N NaOH (57.2 ml; 57.2 mmol; 2.0 eq.). The resulting yellow solution was further stirred, at rt, for 1.5 h. MeOH was then removed under reduced pressure, and the mixture was acidified with aq. 2N HCl (30 ml). Water (40 ml), and DCM (200 ml) were added, and the aq. layer was further extracted with DCM (100 ml). The mixed slightly yellow organic layers were dried over anh. MgSO₄, filtered, and concentrated to dryness under reduced pressure to afford 3-(4-difluoromethoxy-3-fluoro-phenyl)-propionic acid as a beige solid which was further dried under HV (6.680 g; 99%). LC-MS: t_{R}=0.91 min.; [M+H]⁺: no ionisation.

3-(2,3-difluoro-4-trifluoromethyl-phenyl)-propionic acid

According to the described general procedure (GP5), saponification (rt; 1.5 h) of 3-(2,3-difluoro-4-trifluoromethyl-phenyl)-propionic acid butyl ester (13.080 g; 42.15 mmol) afforded 3-(2,3-difluoro-4-trifluoromethyl-phenyl)-propionic acid as a colorless solid (10.640 g; 99%). LC-MS: t_{R}=0.96 min.; [M+H]⁺: no ionisation.

### A.2 Synthesis of Alcohols R¹—CH₂—CH₂—CH₂—OH

A.2.1 Synthesis of Alcohols R¹—CH₂—CH₂—CH₂—OH Via Reduction of the Corresponding carboxylic acids
3-(3-fluoro-4-trifluoromethyl-phenyl)-propan-1-ol
[General Procedure for Reduction of carboxylic acids to Alcohols (GP6)]

An ice-cooled solution of 3-(3-fluoro-4-trifluoromethyl-phenyl)-propionic acid (19.260 g; 81.55 mmol; 1.0 eq.) in anh. THF (120 ml) was treated dropwise (over 20 min.) with a solution of 1M BH₃.THF in THF (122 ml; 122 mmol; 1.5 eq.). The resulting solution was further stirred at 0° C., under nitrogen, for 1 h, and then at rt for 14 h. The resulting reaction mixture was cooled to 0° C., and MeOH (100 ml) was carefully added followed by water (100 ml). MeOH and THF were then removed under reduced pressure. After extraction with DCM (3×100 ml), the combined organic extracts were washed with brine (100 ml), dried over anh. MgSO₄, filtered, and concentrated to dryness under reduced pressure. The crude was purified by FC (DCM/MeOH=9/1) to give 3-(3-fluoro-4-trifluoromethyl-phenyl)-propan-1-ol as a pale yellow oil (17.690 g; 98%). LC-MS: t_{R}=0.90 min.; [M+H]⁺: no ionisation.

3-(4-fluoro-3-trifluoromethyl-phenyl)-propan-1-ol

According to the described general procedure (GP6), borane-mediated reduction of 3-(4-fluoro-3-trifluoromethyl-phenyl)-propionic acid (10.150 g; 42.97 mmol) gave after purification by FC (DCM/MeOH=9/1) 3-(4-fluoro-3-trifluoromethyl-phenyl)-propan-1-ol as a pale yellow oil (8.890 g; 93%). LC-MS: t_{R}=0.95 min.; [M+H]⁺: no ionisation.

3-(4-chloro-3-fluoro-phenyl)-propan-1-ol

According to the described general procedure (GP6), borane-mediated reduction of 3-(4-chloro-3-fluoro-phenyl)-propionic acid (6.000 g; 29.61 mmol) gave after purification by FC (DCM/MeOH=95/5) 3-(4-chloro-3-fluoro-phenyl)-propan-1-ol as a colorless oil (3.170 g; 57%). LC-MS: t_{R}=0.92 min.; [M+H]⁺: no ionisation.

3-(4-difluoromethoxy-3-fluoro-phenyl)-propan-1-ol

According to the described general procedure (GP6), borane-mediated reduction of 3-(4-difluoromethoxy-3-fluoro-phenyl)-propionic acid (6.160 g; 26.30 mmol) gave after purification by FC (DCM/MeOH=15/1) 3-(4-difluoromethoxy-3-fluoro-phenyl)-propan-1-ol as a slightly yellow oil (5.754 g; 99%). LC-MS: t_{R}=0.92 min.; [M+H]⁺: no ionisation.

3-(4-trifluoromethyl-phenyl)-propan-1-ol

According to the described general procedure (GP6), borane-mediated reduction of 3-(4-trifluoromethyl-phenyl)-propionic acid (22.700 g; 98.84 mmol) gave after purification by FC (DCM/MeOH=9/1) 3-(4-trifluoromethyl-phenyl)-propan-1-ol as a colorless oil (20.090 g; 99%). LC-MS: t_{R}=0.94 min.; [M+H]⁺: no ionisation.

3-(4-difluoromethoxy-phenyl)-propan-1-ol

According to the described general procedure (GP6), borane-mediated reduction of 3-(4-difluoromethoxy-phenyl)-propionic acid (13.920 g; 64.39 mmol) gave after purification by FC (DCM/MeOH=9/1) 3-(4-difluoromethoxy-phenyl)-propan-1-ol as a pale yellow oil (11.520 g; 88%). LC-MS: t_{R}=0.90 min.; [M+H]⁺: no ionisation.

3-(2,3-difluoro-4-trifluoromethyl-phenyl)-propan-1-ol

According to the described general procedure (GP6), borane-mediated reduction of 3-(2,3-difluoro-4-trifluoromethyl-phenyl)-propionic acid (6.100 g; 24.00 mmol) gave after purification by FC (DCM/MeOH=15/1) 3-(2,3-difluoro-4-trifluoromethyl-phenyl)-propan-1-01 as a slightly yellow oil (5.650 g; 98%). LC-MS: t_{R}=0.97 min.; [M+H]⁺: no ionisation.

3-(3-difluoromethoxy-phenyl)-propan-1-ol

According to the described general procedure (GP6), borane-mediated reduction of 3-(3-difluoromethoxy-phenyl)-propionic acid (17.880 g; 82.70 mmol) gave after purification by FC (DCM/MeOH=9/1) 3-(3-difluoromethoxy-phenyl)-propan-1-ol as a colorless oil (15.860 g; 95%). LC-MS: t_{R}=0.91 min.; [M+H]⁺: no ionisation.

3-(3-trifluoromethyl-phenyl)-propan-1-ol

According to the described general procedure (GP6), borane-mediated reduction of 3-(3-trifluoromethyl-phenyl)-propionic acid (12.390 g; 56.79 mmol) gave after purification by FC (DCM/MeOH=9/1) 3-(3-trifluoromethyl-phenyl)-propan-1-ol as a pale yellow oil (10.970 g; 94%). LC-MS: t_{R}=0.88 min.; [M+H]⁺: no ionisation.

3-(2-fluoro-4-trifluoromethyl-phenyl)-propan-1-ol

According to the described general procedure (GP6), borane-mediated reduction of 3-(2-fluoro-4-trifluoromethyl-phenyl)-propionic acid (3.159 g; 13.37 mmol) gave after purification by FC (DCM/MeOH=9/1) 3-(2-fluoro-4-trifluoromethyl-phenyl)-propan-1-ol as a colorless oil (2.674 g; 90%). LC-MS: t_{R}=0.95 min.; [M+H]⁺: no ionisation.

### A.2.2 Synthesis of Alcohols R¹—CH₂—CH₂—CH₂—OH Via Reduction of the Corresponding Esters

3-(3-fluoro-4-trifluoromethoxy-phenyl)-propan-1-ol

### [General Procedure for Reduction of Esters to Primary Alcohols (GP7)]

To an ice-cooled solution of 3-(3-fluoro-4-trifluoromethoxy-phenyl)-propionic acid butyl ester (15.880 g; 51.51 mmol; 1.0 eq.) in anh. THF (150 ml) was added dropwise a solution of 1N BH₃.THF in THF (77.3 ml; 77.3 mmol; 1.5 eq.). The resulting solution was further stirred at 0° C., under nitrogen, for 15 min., and then at rt for 15 h. The reaction mixture was cooled to 0° C., and treated dropwise successively with MeOH (50 ml), and water (75 ml). The organic solvents were removed under reduced pressure, and the resulting aq. layer was extracted with DCM (3×150 ml). The combined organic extracts were washed with brine (200 ml), dried over anh. MgSO₄, filtered, and concentrated to dryness under reduced pressure. The crude was purified by FC (DCM/MeOH, 9/1) to give 3-(3-fluoro-4-trifluoromethoxy-phenyl)-propan-1-ol as a colorless oil which was further dried under HV (11.810 g; 96%). LC-MS: t_{R}=0.97 min.; [M+H]⁺: no ionisation.

A.3 Synthesis of aldehydes R¹—CH₂—CH₂—CHO
3-(3-fluoro-4-trifluoromethyl-phenyl)-propionaldehyde

### [General Procedure for the Oxidation of Primary Alcohols to Aldehydes (GP8)]

An ice-cooled orange suspension of pyridinium chlorochromate (4.284 g; 19.87 mmol; 1.5 eq.) in anh. DCM (35 ml) was treated dropwise (over 10 min.) with a solution of 3-(3-fluoro-4-trifluoromethyl-phenyl)-propan-1-ol (3.200 g; 13.25 mmol; 1.0 eq.) in anh. DCM (10 ml). The resulting black suspension was allowed to warm-up to rt, and was further stirred at rt, under nitrogen, for 14 h. The black heterogeneous reaction mixture was directly filtered over silicagel using DCM. Subsequent concentration to dryness under reduced pressure afforded 3-(3-fluoro-4-trifluoromethyl-phenyl)-propionaldehyde as a pale yellow oil (2.195 g; 75%). LC-MS: t_{R}=1.02 min.; [M+H]⁺: no ionisation. The obtained aldehyde was directly introduced in the next reaction.

3-(4-fluoro-3-trifluoromethyl-phenyl)-propionaldehyde

According to the described general procedure (GP8), PCC-mediated oxidation of 3-(4-fluoro-3-trifluoromethyl-phenyl)-propan-1-ol (1.500 g; 6.75 mmol) afforded after filtration over silicagel 3-(4-fluoro-3-trifluoromethyl-phenyl)-propionaldehyde as a slightly yellow oil (1.130 g; 76%).

3-(4-chloro-3-fluoro-phenyl)-propionaldehyde

According to the described general procedure (GP8), PCC-mediated oxidation of 3-(4-chloro-3-fluoro-phenyl)-propan-1-ol (1.000 g; 5.30 mmol) afforded after filtration over silicagel 3-(4-chloro-3-fluoro-phenyl)-propionaldehyde as a colorless oil (780 mg; 79%).

3-(4-difluoromethoxy-phenyl)-propionaldehyde

According to the described general procedure (GP8), PCC-mediated oxidation of 3-(4-difluoromethoxy-phenyl)-propan-1-ol (1.500 g; 7.41 mmol) afforded after filtration over silicagel 3-(4-difluoromethoxy-phenyl)-propionaldehyde as a slightly yellow oil (1.200 g; 81%).

3-(3-fluoro-4-trifluoromethoxy-phenyl)-propionaldehyde

According to the described general procedure (GP8), PCC-mediated oxidation of 3-(3-fluoro-4-trifluoromethoxy-phenyl)-propan-1-ol (1.400 g; 5.87 mmol) afforded after filtration over silicagel 3-(3-fluoro-4-trifluoromethoxy-phenyl)-propionaldehyde as a colorless oil (1.020 g; 73%).

3-(4-difluoromethoxy-3-fluoro-phenyl)-propionaldehyde

According to the described general procedure (GP8), PCC-mediated oxidation of 3-(4-difluoromethoxy-3-fluoro-phenyl)-propan-1-ol (1.370 g; 6.22 mmol) afforded after filtration over silicagel 3-(4-difluoromethoxy-3-fluoro-phenyl)-propionaldehyde as a pale yellow oil (820 mg; 60%).

3-(4-trifluoromethyl-phenyl)-propionaldehyde

According to the described general procedure (GP8), PCC-mediated oxidation of 3-(4-trifluoromethyl-phenyl)-propan-1-ol (2.000 g; 9.79 mmol) afforded after filtration over silicagel 3-(4-trifluoromethyl-phenyl)-propionaldehyde as a pale yellow oil (1.410 g; 71%).

3-(2,3-difluoro-4-trifluoromethyl-phenyl)-propionaldehyde

According to the described general procedure (GP8), PCC-mediated oxidation of 3-(2,3-difluoro-4-trifluoromethyl-phenyl)-propan-1-ol (1.890 g; 7.86 mmol) afforded after filtration over silicagel 3-(2,3-difluoro-4-trifluoromethyl-phenyl)-propionaldehyde as a pale yellow oil (940 mg; 50%).

3-(3-difluoromethoxy-phenyl)-propionaldehyde

According to the described general procedure (GP8), PCC-mediated oxidation of 3-(3-difluoromethoxy-phenyl)-propan-1-ol (1.500 g; 7.41 mmol) afforded after filtration over silicagel 3-(3-difluoromethoxy-phenyl)-propionaldehyde as a colorless oil (1.140 g; 77%).

3-(3-trifluoromethyl-phenyl)-propionaldehyde

According to the described general procedure (GP8), PCC-mediated oxidation of 3-(3-trifluoromethyl-phenyl)-propan-1-ol (3.164 g; 15.50 mmol) afforded after filtration over silicagel 3-(3-trifluoromethyl-phenyl)-propionaldehyde as a colorless oil (2.450 g; 78%). LC-MS: t_{R}=1.02 min.; [M+H]⁺: no ionisation.

3-(2-fluoro-4-trifluoromethyl-phenyl)-propionaldehyde

According to the described general procedure (GP8), PCC-mediated oxidation of 3-(2-fluoro-4-trifluoromethyl-phenyl)-propan-1-ol (3.443 g; 15.50 mmol) afforded after filtration over silicagel 3-(2-fluoro-4-trifluoromethyl-phenyl)-propionaldehyde as a colorless oil (2.550 g; 75%). LC-MS: t_{R}=1.00 min.; [M+H]⁺: no ionisation.

### B. Synthesis of Substituted Imidazoles

B.1 CuCl-Mediated Synthesis of Substituted Imidazoles from Nitriles and α-Amino Acetals
2-cyclopropyl-4,5-diiodo-1H-imidazole
[General Procedure for the Cucl-Mediated Synthesis of Substituted Imidazoles from Nitriles and α-Amino Acetals (GP9)]

Ice-cooled aminoacetaldehyde dimethylacetal (16.0 ml; 146.85 mmol) was treated successively (in one portion) with cyclopropyl cyanide (13.5 ml; 183.57 mmol), and cuprous chloride CuCl (18.171 g; 183.57 mmol), and the resulting green heterogeneous mixture was heated to 85° C., under nitrogen, for 14 h 30. MeOH (40 ml), and thioacetamide (13.791 g; 183.57 mmol) were then added to the ice-cooled reaction mixture. Subsequent heating at 45° C. for 1 h, filtration of the dark-brown heterogeneous mixture over a pad of celite, and washing of the separated solid with MeOH (90 ml) afforded a yellow/orange homogeneous filtrate which was cooled (0° C.), and treated dropwise with concentrated 12N hydrochloric acid (26.4 ml). The resulting mixture was then heated to 80° C., under nitrogen, for 3 h 45. MeOH was removed under reduced pressure, and a solution of NaOH (14.00 g; 350.00 mmol) in water (28 ml) was added portionwise to the ice-cooled mixture. Dioxane (100 ml), water (60 ml), Na₂CO₃ (46.70 g; 440.57 mmol), and finally iodine (82.00 g; 323.08 mmol) were successively added in one portion, at rt, to the reaction mixture which was then further stirred at rt, under nitrogen, for 14 h 30. A solution of sodium thiosulfate (63.00 g) in water (400 ml), and AcOEt (400 ml) were successively added to the resulting reaction mixture. The dark-brown organic layer was further washed with brine (2×100 ml), and the combined aq. layers were further extracted with AcOEt (200 ml). The mixed organic layers were then dried over anh. MgSO₄, filtered, and concentrated to dryness under reduced pressure. The crude black oil (21.71 g) was purified by FC (DCM/MeOH/25% aq. NH₄OH=200/10/1) to give 2-cyclopropyl-4,5-diiodo-1H-imidazole as a beige solid which was further dried under HV (11.840 g; 22% overall yield). LC-MS: t_{R}=0.66 min.; [M+H]⁺: 361.07 g/mol.

2-cyclopropylmethyl-4,5-diiodo-1H-imidazole

According to the described general procedure (GP9), the target product was obtained as slightly beige solid by reaction between cyclopropyl-acetonitrile (17.0 ml; 183.57 mmol), and aminoacetaldehyde dimethylacetal (16.0 ml; 146.85 mmol) followed by cyclization to the corresponding imidazole, iodination, and finally purification by FC (DCM/MeOH/25% aq. NH₄OH=200/10/1). LC-MS: t_{R}=0.71 min.; [M+H]⁺: 374.86 g/mol.

### B.2 Polyiodination of Imidazoles

2-ethyl-4,5-diiodo-1H-imidazole

### [General Procedure for Diiodination of Imidazoles (GP10)]

To a solution of 2-ethylimidazole (15.000 g; 156.03 mmol; 1.0 eq.) in dioxane (250 ml), and water (250 ml) was added at rt (in one portion) successively Na₂CO₃ (49.614 g; 468.10 mmol; 3.0 eq.), and iodine (87.126 g; 343.27 mmol; 2.2 eq.). The resulting brown heterogeneous reaction mixture was further stirred at rt, under nitrogen, for 24 h. AcOEt (500 ml) was then added followed by an aq. solution of sodium thiosulfate (45.0 g Na₂S₂O₃ in 300 ml of water). The yellow organic layer was separated, and additionally washed with an aq. solution of sodium thiosulfate (30.0 g Na₂S₂O₃ in 300 ml of water), and finally with brine (200 ml). The resulting yellow organic layer was then dried over anh. MgSO₄, filtered, and concentrated to dryness under reduced pressure to give 2-ethyl-4,5-diiodo-1H-imidazole as a pale yellow solid which was further dried under HV (49.760 g; 92%). LC-MS: t_{R}=0.55 min.; [M+H]⁺: 349.18 g/mol.

4,5-diiodo-2-methyl-1H-imidazole

According to the described general procedure (GP10), diiodination of 2-methyl-1H-imidazole (15.000 g; 182.68 mmol) afforded 4,5-diiodo-2-methyl-1H-imidazole as a yellow solid (61.00 g; 100%). LC-MS: t_{R}=0.52 min.; [M+H]⁺: 335.14 g/mol.

2,4,5-triiodo-1H-imidazole

According to the described general procedure (GP10), triiodination (rt; 15 h) of imidazole (8.000 g; 117.51 mmol; 1.0 eq.) with iodine (98.424 g; 387.78 mmol; 3.3 eq.) and sodium carbonate (56.047 g; 528.79 mmol; 4.5 eq.) in a mixture of dioxane (300 ml) and water (300 ml) afforded 2,4,5-triiodo-1H-imidazole as a yellow solid (53.620 g; 100%). LC-MS: t_{R}=0.84 min.; [M+H]⁺: 446.66 g/mol.

B.3 N-alkylation of imidazoles with Br(CH₂)₂NHBoc or with (2-bromo-ethyl)-carbamic acid benzyl ester
Preparation of (2-bromo-ethyl)-carbamic acid benzyl ester

A cooled (0° C.) mixture of 2-bromoethylamine hydrobromide (12.000 g; 58.56 mmol; 1.0 eq.) in dioxane (60 ml) was treated with aq. 1M NaOH (117.2 ml; 117.20 mmol; 2.0 eq.), and dropwise (over 10 min.) with benzyl chloroformate (8.4 ml; 58.8 mmol; 1.0 eq.). The resulting mixture was further stirred at 0° C., under nitrogen, for 10 min., and then at rt for 13 h. Et₂O (300 ml) was added, and the colorless organic layer was further washed with water (75 ml), dried over anh. MgSO₄, filtered, and finally concentrated to dryness under reduced pressure to afford (2-bromo-ethyl)-carbamic acid benzyl ester as a colorless oil which was further dried under HV (15.020 g; 99%). LC-MS: t_{R}=0.95 min.; [M+H]⁺: no ionisation.

[2-(2-cyclopropyl-4,5-diiodo-imidazol-1-yl)-ethyl]carbamic acid tert-butyl ester

### [General Procedure for N-Alkylation of Imidazoles (GP11)]

To a solution of 2-cyclopropyl-4,5-diiodo-1H-imidazole (11.840 g; 32.89 mmol; 1.0 eq.) in anh. DMF (160 ml) was added portionwise (over 2 min.), at rt, 55-65% NaH moistened with oil (1.579 g; 39.48 mmol; 1.2 eq.), and stirring at rt, under nitrogen, was continued for 20 min. The mixture was then heated to 100° C., and a colorless homogeneous solution of Br(CH₂)₂NHBoc (8.109 g; 36.18 mmol; 1.1 eq.) in anh. DMF (90 ml) was added dropwise (over 1 h). After completion of the addition, the resulting brown homogeneous mixture was further heated at 100° C. for 1 h 30. The reaction mixture was allowed to cool to rt, and water (550 ml) was added dropwise. This mixture was extracted with Et₂O (4×200 ml), and the mixed organic layers were dried over anh. MgSO₄, filtered, and concentrated to dryness under reduced pressure to give a brown oil (22.560 g). The crude was purified by FC (DCM/MeOH=25/1) to give [2-(2-cyclopropyl-4,5-diiodo-imidazol-1-yl)-ethyl]-carbamic acid tert-butyl ester as a beige solid which was further dried under HV (10.870 g; 66%). LC-MS: t_{R}=0.87 min.; [M+H]⁺: 504.11 g/mol.

[2-(2-ethyl-4,5-diiodo-imidazol-1-yl)-ethyl]carbamic acid tert-butyl ester

According to the described general procedure (GP11), N-alkylation of 2-ethyl-4,5-diiodo-1H-imidazole (10.000 g; 28.74 mmol) with Br(CH₂)₂NHBoc, and subsequent purification by FC (DCM/MeOH=25/1) afforded [2-(2-ethyl-4,5-diiodo-imidazol-1-yl)-ethyl]-carbamic acid tert-butyl ester as a pale yellow solid (9.950 g; 70%). LC-MS: t_{R}=0.78 min.; [M+H]⁺: 492.33 g/mol.

[2-(4,5-diiodo-2-methyl-imidazol-1-yl)-ethyl]carbamic acid tert-butyl ester

According to the described general procedure (GP11), N-alkylation of 4,5-diiodo-2-methyl-1H-imidazole (5.000 g; 14.97 mmol) with Br(CH₂)₂NHBoc, and subsequent purification by FC (DCM/MeOH=10/1) afforded [2-(4,5-diiodo-2-methyl-imidazol-1-yl)-ethyl]-carbamic acid tert-butyl ester as a yellow solid (4.400 g; 62%). LC-MS: t_{R}=0.74 min.; [M+H]⁺: 478.28 g/mol.

[2-(4-methyl-imidazol-1-yl)-ethyl]carbamic acid benzyl ester

According to the described general procedure (GP11), N-alkylation of commercially available 4(5)-methylimidazole (4.343 g; 52.90 mmol) with (2-bromo-ethyl)-carbamic acid benzyl ester (15.019 g; 58.19 mmol), and subsequent purification by FC (DCM/MeOH=10/1) afforded a mixture of [2-(4-methyl-imidazol-1-yl)-ethyl]-carbamic acid benzyl ester, and [2-(5-methyl-imidazol-1-yl)ethyl]-carbamic acid benzyl ester (ratio of regioisomers close to 1/1, according to ¹H-NMR) as a yellow oil (4.270 g; 31%). LC-MS: t_{R}=0.68 min. (2 regioisomers); [M+H]⁺: 260.46 g/mol.

[2-(2-methylsulfanyl-imidazol-1-yl)-ethyl]carbamic acid tert-butyl ester

An ice-cooled yellow heterogeneous mixture of 1H-imidazole-2-thiol (15.570 g; 155.47 mmol) in anh. acetone (325 ml) was treated with anh. K₂CO₃ (21.488 g; 155.47 mmol), and stirring at 0° C., under nitrogen, was continued for 15 min. The resulting mixture was then treated dropwise (over 45 min.) with a solution of iodomethane (10.67 ml; 171.02 mmol) in anh. acetone (45 ml), and the resulting beige suspension was further stirred at 0° C. for 1 h 15, and then at rt for 15 h. The heterogeneous reaction mixture was filtered, and the discarded salts were washed with acetone. The filtrate was then concentrated to dryness under reduced pressure to give the crude 2-methylsulfanyl-1H-imidazole as a beige solid which was further dried under HV (25.080 g). LC-MS: t_{R}=0.23 min.; [M+H]⁺: no ionisation.

This crude 2-methylsulfanyl-1H-imidazole was then dissolved in anh. DMF (300 ml), and was treated portionwise (over 2 min.), at rt, with 55-65% NaH moistened with oil (7.462 g; 186.57 mmol). Stirring at rt, under nitrogen, was continued for 20 min. The mixture was then heated to 100° C., and a colorless solution of Br(CH₂)₂NHBoc (38.326 g; 171.02 mmol) in anh. DMF (100 ml) was added dropwise (over 1 h). After completion of the addition, the resulting mixture was further heated at 100° C. for 45 min. The reaction mixture was allowed to cool to rt, and water (800 ml) was added portionwise. This mixture was extracted with Et₂O (3×400 ml), and the mixed organic layers were dried over anh. MgSO₄, filtered, and concentrated to dryness under reduced pressure. Residual DMF was removed under HV. The crude orange oil (20.050 g) was purified by FC (DCM/MeOH=10/1) to afford [2-(2-methylsulfanyl-imidazol-1-yl)-ethyl]-carbamic acid tert-butyl ester as a yellow oil which was further dried under HV (12.420 g; 31% for the two steps). LC-MS: t_{R}=0.64 min.; [M+H]⁺: 258.33 g/mol.

[2-(2-cyclopropylmethyl-4,5-diiodo-imidazol-1-yl)-ethyl]-carbamic acid tert-butyl ester

According to the described general procedure (GP11), N-alkylation of 2-cyclopropylmethyl-4,5-diiodo-1H-imidazole (3.770 g; 10.08 mmol) with Br(CH₂)₂NHBoc (2.485 g; 11.09 mmol), and subsequent purification by FC (DCM/MeOH=50/1) afforded [2-(2-cyclopropylmethyl-4,5-diiodo-imidazol-1-yl)-ethyl]-carbamic acid tert-butyl ester as a beige solid (3.980 g; 76%). LC-MS: t_{R}=0.88 min.; [M+H]⁺: 518.16 g/mol.

[2-(2,4,5-triiodo-imidazol-1-yl)-ethyl]carbamic acid tert-butyl ester

According to the described general procedure (GP11), N-alkylation of 2,4,5-triiodo-1H-imidazole (65.180 g; 146.22 mmol) with Br(CH₂)₂NHBoc (36.045 g; 160.84 mmol), and subsequent purification by FC (AcOEt/heptane=2/3) afforded [2-(2,4,5-triiodo-imidazol-1-yl)-ethyl]-carbamic acid tert-butyl ester as a colorless solid (36.500 g; 42%). LC-MS: t_{R}=0.85 min.; [M+H]⁺: 589.76 g/mol.

B.4 Regioselective Deiodination of Imidazoles with Grignard Reagents
[2-(2-cyclopropyl-4-iodo-imidazol-1-yl)-ethyl]-carbamic acid tert-butyl ester

### [General Procedure for Regioselective Deiodination of Imidazoles (GP12)]

A cooled (−40° C.) solution of [2-(2-cyclopropyl-4,5-diiodo-imidazol-1-yl)-ethyl]-carbamic acid tert-butyl ester (10.870 g; 21.60 mmol) in anh. THF (600 ml) was treated dropwise (over 15 min.) with a solution of 3M EtMgBr in Et₂O (7.2 ml; 21.60 mmol). After addition, the resulting orange homogeneous solution was further stirred at −40° C., under nitrogen, for 30 min. (conversion reached 42% according to LC-MS), and additional 3M EtMgBr in Et₂O (6.0 ml; 18 mmol) was then added to the cooled reaction mixture until the reaction was completed. The reaction mixture was then treated at −40° C. with aq. sat. NH₄Cl (20 ml), and was allowed to warm-up to rt. Et₂O (250 ml) was added, and the resulting solution was washed successively with water (200 ml), and brine (200 ml). The yellow organic layer was dried over anh. MgSO₄, filtered, and concentrated to dryness under reduced pressure to give an orange oil (9.580 g). The crude was purified by FC (DCM/MeOH=20/1) to afford [2-(2-cyclopropyl-4-iodo-imidazol-1-yl)-ethyl]-carbamic acid tert-butyl ester as a yellow oil which was further dried under HV (7.810 g; 96%). LC-MS: t_{R}=0.70 min.; [M+H]⁺: 378.32 g/mol.

[2-(2-ethyl-4-iodo-imidazol-1-yl)-ethyl]carbamic acid tert-butyl ester

According to the described general procedure (GP12), regioselective deiodination of [2-(2-ethyl-4,5-diiodo-imidazol-1-yl)-ethyl]-carbamic acid tert-butyl ester (22.990 g; 46.81 mmol), and subsequent purification by FC (DCM/MeOH=20/1) afforded [2-(2-ethyl-4-iodo-imidazol-1-yl)-ethyl]-carbamic acid tert-butyl ester as a yellow solid (15.500 g; 91%). LC-MS: t_{R}=0.65 min.; [M+H]⁺: 366.39 g/mol.

[2-(4-iodo-2-methyl-imidazol-1-yl)-ethyl]carbamic acid tert-butyl ester

According to the described general procedure (GP12), regioselective deiodination of [2-(4,5-diiodo-2-methyl-imidazol-1-yl)-ethyl]-carbamic acid tert-butyl ester (13.300 g; 27.87 mmol), and subsequent purification by FC (DCM/MeOH=15/1) afforded [2-(4-iodo-2-methyl-imidazol-1-yl)-ethyl]-carbamic acid tert-butyl ester as a yellow solid (7.270 g; 74%). LC-MS: t_{R}=0.62 min.; [M+H]⁺: 352.34 g/mol.

[2-(2-cyclopropylmethyl-4-iodo-imidazol-1-yl)-ethyl]carbamic acid tert-butyl ester

According to the described general procedure (GP12), regioselective deiodination of [2-(2-cyclopropylmethyl-4,5-diiodo-imidazol-1-yl)-ethyl]-carbamic acid tert-butyl ester (3.980 g; 7.69 mmol), and subsequent purification by FC (DCM/MeOH=20/1) afforded [2-(2-cyclopropylmethyl-4-iodo-imidazol-1-yl)-ethyl]-carbamic acid tert-butyl ester as a yellow oil (2.470 g; 82%). LC-MS: t_{R}=0.72 min.; [M+H]⁺: 391.97 g/mol.

[2-(4-iodo-imidazol-1-yl)-ethyl]carbamic acid tert-butyl ester

According to the described general procedure (GP12), regioselective deiodination of [2-(2,4,5-triiodo-imidazol-1-yl)-ethyl]-carbamic acid tert-butyl ester (38.770 g; 65.83 mmol; 1.0 eq.) with 3M EtMgBr in Et₂O (51.63 ml; 154.89 mmol; 2.35 eq.), and subsequent purification by FC (DCM/MeOH=20/1) afforded [2-(4-iodo-imidazol-1-yl)-ethyl]-carbamic acid tert-butyl ester as a colorless solid (14.290 g; 64%). LC-MS: t_{R}=0.67 min.; [M+H]⁺: 338.26 g/mol.

B.5 Preparation of 2-(imidazol-1-yl)-ethylamine derivatives from the Corresponding carbamates
2-(2-cyclopropyl-4-iodo-imidazol-1-yl)-ethylamine

### [General Procedure for Cleavage of Boc-Group (GP13)]

To an ice-cooled solution of [2-(2-cyclopropyl-4-iodo-imidazol-1-yl)-ethyl]carbamic acid tert-butyl ester (7.810 g; 20.70 mmol; 1.0 eq.) in DCM (160 ml) was added dropwise (over 30 min.) a solution of 4N HCl in 1,4-dioxane (51.8 ml; 207.04 mmol; 10.0 eq.). The resulting beige suspension was further stirred at 0° C. for 15 min., and then at rt for 1 h 30. The volatiles were removed under reduced pressure, and 2-(2-cyclopropyl-4-iodo-imidazol-1-yl)-ethylamine was additionally dried under HV to afford a beige solid (7.140 g; 100%; presence of 1.9 eq. of HCl). LC-MS: t_{R}=0.15-0.25 min. (broad peak); [M+H]⁺: 278.13 g/mol.

2-(2-methylsulfanyl-imidazol-1-yl)-ethylamine

According to the described general procedure (GP13), HCl-mediated deprotection (rt; 2.5 h) of [2-(2-methylsulfanyl-imidazol-1-yl)-ethyl]-carbamic acid tert-butyl ester (12.420 g; 48.26 mmol) afforded the chlorhydrate salt of 2-(2-methylsulfanyl-imidazol-1-yl)-ethylamine as a slightly beige solid (10.580 g; 100%; presence of 2 eq. HCl). LC-MS: t_{R}=0.15-0.18 min. (broad peak); [M+H]⁺: 158.20 g/mol.

2-(2-ethyl-4-iodo-imidazol-1-yl)-ethylamine

According to the described general procedure (GP13), HCl-mediated deprotection (rt; 1 h) of [2-(2-ethyl-4-iodo-imidazol-1-yl)-ethyl]carbamic acid tert-butyl ester (5.720 g; 15.66 mmol) afforded the chlorhydrate salt of 2-(2-ethyl-4-iodo-imidazol-1-yl)-ethylamine as a pale beige solid (5.960 g; 100%; presence of 3 eq. HCl). LC-MS: t_{R}=0.14 min.; [M+H]⁺: 266.24 g/mol.

2-(4-iodo-2-methyl-imidazol-1-yl)-ethylamine

According to the described general procedure (GP13), HCl-mediated deprotection (rt; 1 h) of [2-(4-iodo-2-methyl-imidazol-1-yl)-ethyl]carbamic acid tert-butyl ester (2.800 g; 7.97 mmol) afforded the chlorhydrate salt of 2-(4-iodo-2-methyl-imidazol-1-yl)-ethylamine as a pale beige solid (2.880 g; 100%; presence of 3 eq. HCl). LC-MS: t_{R}=0.14 min.; [M+H]⁺: 251.92 g/mol.

2-(2-cyclopropylmethyl-4-iodo-imidazol-1-yl)-ethylamine

According to the described general procedure (GP13), HCl-mediated deprotection (rt; 45 min.) of [2-(2-cyclopropylmethyl-4-iodo-imidazol-1-yl)-ethyl]carbamic acid tert-butyl ester (2.470 g; 6.31 mmol) afforded the chlorhydrate salt of 2-(2-cyclopropylmethyl-4-iodo-imidazol-1-yl)-ethylamine as a beige solid (2.460 g; 100%; presence of 2 eq. HCl). LC-MS: t_{R}=0.23-0.30 min. (broad peak); [M+H]⁺: 292.24 g/mol.

2-(4-methyl-imidazol-1-yl)-ethylamine

A mixture of [2-(4-methyl-imidazol-1-yl)-ethyl]carbamic acid benzyl ester, [2-(5-methyl-imidazol-1-yl)-ethyl]-carbamic acid benzyl ester (1.198 g; 4.62 mmol; ratio of regioisomers close to 1/1, according to ¹H-NMR), and 10% palladium on activated charcoal (240 mg; 20% in mass) was placed under nitrogen before addition of anh. MeOH (20 ml). The resulting mixture was placed under vacuum, and then under hydrogen (1 atm), and stirring at rt was continued for 2.5 h. Filtration over a pad of celite, concentration to dryness under reduced pressure afforded a mixture of 2-(4-methyl-imidazol-1-yl)-ethylamine, and 2-(5-methyl-imidazol-1-yl)-ethylamine as a slightly yellow oil (540 mg; 93%). LC-MS: t_{R}=0.17 min. (2 regioisomers); [M+H]⁺: no ionisation.

These primary amines were converted to the corresponding chlorhydrate salt by treatment of a solution of regioisomeric amines (540 mg; 4.31 mmol) in dichloromethane (5 ml) with 4N HCl in 1,4-dioxane (3.25 ml; 3 eq.). Concentration to dryness under reduced pressure afforded a beige solid which was further dried under HV.

2-(4-iodo-imidazol-1-yl)-ethylamine

According to the described general procedure (GP13), HCl-mediated deprotection (rt; 2 h) of [2-(4-iodo-imidazol-1-yl)-ethyl]-carbamic acid tert-butyl ester (14.290 g; 42.38 mmol) afforded the chlorhydrate salt of 2-(4-iodo-imidazol-1-yl)-ethylamine as a pale yellow solid (13.500 g; 100%; presence of 2 eq. HCl). LC-MS: t_{R}=0.19 min. (broad peak); [M+H]⁺: 238.23 g/mol.

2-(4-ethyl-imidazol-1-yl)-ethylamine

According to the described general procedure (GP13), HCl-mediated deprotection (rt; 48 h) of [2-(4-ethyl-imidazol-1-yl)-ethyl]-carbamic acid tert-butyl ester (1.960 g; 8.19 mmol) afforded the chlorhydrate salt of 2-(4-ethyl-imidazol-1-yl)-ethylamine as an orange solid (1.780 g; 100%; presence of 2 eq. HCl). LC-MS: t_{R}=0.17 min.

### C. Synthesis of 5,6,7,8-tetrahydro-imidazo[1,5-a]pyrazine Derivatives

C.1 Synthesis of 5,6,7,8-tetrahydro-imidazo[1,5-a]pyrazine derivatives Via Microwave-Assisted Pictet-Spengler Reaction
3-cyclopropyl-8-[2-(4-difluoromethoxy-phenyl)-ethyl]-1-iodo-5,6-dihydro-8H-imidazo[1,5-a]pyrazine-7-carboxylic acid tert-butyl ester

### [General Procedure for Microwave-Assisted Pictet-Spengler Reaction (GP14)]

The microwave-assisted Pictet-Spengler reactions were carried out with a CEM Discover apparatus.

A suspension of chlorhydrate salt of the primary amine 2-(2-cyclopropyl-4-iodo-imidazol-1-yl)-ethylamine (1.000 g; 2.85 mmol; 1.0 eq.; presence of 2 eq. HCl) in anh. EtOH (7 ml) was treated successively with N-ethyldiisopropylamine (1.47 ml; 8.57 mmol; 3 eq.), and with a solution of 3-(4-difluoromethoxy-phenyl)-propionaldehyde (572 mg; 2.85 mmol; 1.0 eq.) in anh. EtOH (7 ml). The resulting homogeneous solution was sealed, and put in the microwave oven (60 W; 140° C.; 7 bars; 10 min.). The resulting reaction mixture was allowed to cool to rt, and was then concentrated to dryness under reduced pressure giving the crude 3-cyclopropyl-8-[2-(4-difluoromethoxy-phenyl)-ethyl]-1-iodo-5,6,7,8-tetrahydro-imidazo[1,5-a]pyrazine as a yellow/orange oil (2.410 g). LC-MS: t_{R}=0.72 min.; [M+H]⁺: 460.09 g/mol.

The crude 3-cyclopropyl-8-[2-(4-difluoromethoxy-phenyl)-ethyl]-1-iodo-5,6,7,8-tetrahydro-imidazo[1,5-a]pyrazine (theoretical amount: 2.85 mmol) was dissolved in anh. DCM (20 ml), and N-ethyldiisopropylamine (0.98 ml; 5.71 mmol; 2.0 eq.) was added. The resulting yellow solution was then cooled to 0° C., and a solution of di-tert-butyl dicarbonate Boc₂O (748 mg; 3.42 mmol; 1.2 eq.) in anh. DCM (10 ml) was added portionwise (over 15 min.). The reaction mixture was further stirred at 0° C. for 15 min., and at rt, under nitrogen, for 16 h. The resulting orange solution was diluted with DCM (70 ml), and was then washed successively with water (35 ml), and with brine (35 ml). The organic layer was dried over anh. MgSO₄, filtered, and concentrated to dryness under reduced pressure. The crude was purified by FC (DCM/MeOH=50/1) to afford 3-cyclopropyl-8-[2-(4-difluoromethoxy-phenyl)-ethyl]-1-iodo-5,6-dihydro-8H-imidazo[1,5-a]pyrazine-7-carboxylic acid tert-butyl ester as a beige solid which was further dried under HV (1.210 g; 76%). LC-MS: t_{R}=0.97 min.; [M+H]⁺: 560.48 g/mol.

3-cyclopropyl-8-[2-(3-fluoro-4-trifluoromethyl-phenyl)-ethyl]-1-iodo-5,6-dihydro-8H-imidazo[1,5-a]pyrazine-7-carboxylic acid tert-butyl ester

According to the general procedure (GP14), microwave-assisted Pictet-Spengler reaction (70 W; 160° C.; 10.0 bars; 10 min.) between 2-(2-cyclopropyl-4-iodo-imidazol-1-yl)-ethylamine (9.01 mmol) and 3-(3-fluoro-4-trifluoromethyl-phenyl)-propionaldehyde (1.770 g; 8.03 mmol) afforded 3-cyclopropyl-8-[2-(3-fluoro-4-trifluoromethyl-phenyl)-ethyl]-1-iodo-5,6,7,8-tetrahydro-imidazo[1,5-a]pyrazine as a brown oil. LC-MS: t_{R}=0.76 min.; [M+H]⁺: 480.30 g/mol.

Subsequent protection of the secondary amine, and purification by FC (DCM/MeOH=25/1) allowed the isolation of 3-cyclopropyl-8-[2-(3-fluoro-4-trifluoromethyl-phenyl)-ethyl]-1-iodo-5,6-dihydro-8H-imidazo[1,5-a]pyrazine-7-carboxylic acid tert-butyl ester as a yellow solid (3.780 g; 72%). LC-MS: t_{R}=0.96 min.; [M+H]⁺: 580.26 g/mol.

3-cyclopropyl-8-[2-(4-fluoro-3-trifluoromethyl-phenyl)-ethyl]-1-iodo-5,6-dihydro-8H-imidazo[1,5-a]pyrazine-7-carboxylic acid tert-butyl ester

According to the general procedure (GP14), microwave-assisted Pictet-Spengler reaction (60 W; 140° C.; 7.0 bars; 10 min.) between 2-(2-cyclopropyl-4-iodo-imidazol-1-yl)-ethylamine (2.85 mmol) and 3-(4-fluoro-3-trifluoromethyl-phenyl)-propionaldehyde (629 mg; 2.85 mmol) afforded 3-cyclopropyl-8-[2-(4-fluoro-3-trifluoromethyl-phenyl)-ethyl]-1-iodo-5,6,7,8-tetrahydro-imidazo[1,5-a]pyrazine. LC-MS: t_{R}=0.80 min.; [M+H]⁺: 479.81 g/mol.

Subsequent protection of the secondary amine, and purification by FC (DCM/MeOH=25/1) allowed the isolation of 3-cyclopropyl-8-[2-(4-fluoro-3-trifluoromethyl-phenyl)-ethyl]-1-iodo-5,6-dihydro-8H-imidazo[1,5-a]pyrazine-7-carboxylic acid tert-butyl ester as a colorless solid (1.420 g; 86%). LC-MS: t_{R}=1.01 min.; [M+H]⁺: 580.18 g/mol.

8-[2-(4-chloro-3-fluoro-phenyl)-ethyl]-3-cyclopropyl-1-iodo-5,6-dihydro-8H-imidazo[1,5-a]pyrazine-7-carboxylic acid tert-butyl ester

According to the general procedure (GP14), microwave-assisted Pictet-Spengler reaction (60 W; 140° C.; 7.0 bars; 10 min.) between 2-(2-cyclopropyl-4-iodo-imidazol-1-yl)-ethylamine (2.85 mmol) and 3-(4-chloro-3-fluoro-phenyl)-propionaldehyde (533 mg; 2.85 mmol) afforded 8-[2-(4-chloro-3-fluoro-phenyl)-ethyl]-3-cyclopropyl-1-iodo-5,6,7,8-tetrahydro-imidazo[1,5-a]pyrazine. LC-MS: t_{R}=0.76 min.; [M+H]⁺: 446.12 g/mol.

Subsequent protection of the secondary amine, and purification by FC (AcOEt/heptane=7/3) allowed the isolation of 8-[2-(4-chloro-3-fluoro-phenyl)-ethyl]-3-cyclopropyl-1-iodo-5,6-dihydro-8H-imidazo[1,5-a]pyrazine-7-carboxylic acid tert-butyl ester as a colorless solid (1.100 g; 71%). LC-MS: t_{R}=0.98 min.; [M+H]⁺: 546.26 g/mol.

3-cyclopropyl-8-[2-(3-fluoro-4-trifluoromethoxy-phenyl)-ethyl]-1-iodo-5,6-dihydro-8H-imidazo[1,5-a]pyrazine-7-carboxylic acid tert-butyl ester

According to the general procedure (GP14), microwave-assisted Pictet-Spengler reaction (60 W; 140° C.; 7.0 bars; 10 min.) between 2-(2-cyclopropyl-4-iodo-imidazol-1-yl)-ethylamine (3.50 mmol) and 3-(3-fluoro-4-trifluoromethoxy-phenyl)-propionaldehyde (1.020 g; 4.31 mmol) afforded 3-cyclopropyl-8-[2-(3-fluoro-4-trifluoromethoxy-phenyl)-ethyl]-1-iodo-5,6,7,8-tetrahydro-imidazo[1,5-a]pyrazine. LC-MS (basic conditions): t_{R}=0.93 min.; [M+H]⁺: 496.01 g/mol.

Subsequent protection of the secondary amine, and purification by FC (AcOEt/heptane=15/85 to 9/1) allowed the isolation of 3-cyclopropyl-8-[2-(3-fluoro-4-trifluoromethoxy-phenyl)-ethyl]-1-iodo-5,6-dihydro-8H-imidazo[1,5-a]pyrazine-7-carboxylic acid tert-butyl ester as a pale yellow solid (1.790 g; 86%). LC-MS (basic conditions): t_{R}=1.07 min.; [M+H]⁺: 596.04 g/mol.

3-cyclopropyl-8-[2-(4-difluoromethoxy-3-fluoro-phenyl)-ethyl]-1-iodo-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 7.0 bars; 10 min.) between 2-(2-cyclopropyl-4-iodo-imidazol-1-yl)-ethylamine (3.13 mmol) and 3-(4-difluoromethoxy-3-fluoro-phenyl)-propionaldehyde (819 mg; 3.75 mmol) afforded 3-cyclopropyl-8-[2-(4-difluoromethoxy-3-fluoro-phenyl)-ethyl]-1-iodo-5,6,7,8-tetrahydro-imidazo[1,5-a]pyrazine. LC-MS: t_{R}=0.77 min.; [M+H]⁺: 478.21 g/mol.

Subsequent protection of the secondary amine, and purification by FC (AcOEt/heptane=1/9 to AcOEt) allowed the isolation of 3-cyclopropyl-8-[2-(4-difluoromethoxy-3-fluoro-phenyl)-ethyl]-1-iodo-5,6-dihydro-8H-imidazo[1,5-a]pyrazine-7-carboxylic acid tert-butyl ester as a pale yellow solid (1.400 g; 77% for two steps). LC-MS: t_{R}=0.98 min.; [M+H]⁺: 578.45 g/mol.

3-methylsulfanyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 6.0 bars; 10 min.) between 2-(2-methylsulfanyl-imidazol-1-yl)-ethylamine (38.77 mmol) and 3-(4-trifluoromethyl-phenyl)-propionaldehyde (7.838 g; 38.77 mmol) afforded 3-methylsulfanyl-8-[2-(4-trifluoromethyl-phenyl)-ethyl]-5,6,7,8-tetrahydro-imidazo[1,5-a]pyrazine. LC-MS: t_{R}=0.71 min.; [M+H]⁺: 342.40 g/mol.

Subsequent protection of the secondary amine, and purification by FC (DCM/MeOH=25/1) allowed the isolation of 3-methylsulfanyl-8-[2-(4-trifluoromethyl-phenyl)-ethyl]-5,6-dihydro-8H-imidazo[1,5-a]pyrazine-7-carboxylic acid tert-butyl ester as a yellow solid (10.780 g; 63% for two steps). LC-MS: t_{R}=0.94 min.; [M+H]⁺: 442.59 g/mol.

8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 6.0 bars; 10 min.) between 2-(2-methylsulfanyl-imidazol-1-yl)-ethylamine (3.28 mmol) and 3-(2,3-difluoro-4-trifluoromethyl-phenyl)-propionaldehyde (939 mg; 3.94 mmol) afforded 8-[2-(2,3-difluoro-4-trifluoromethyl-phenyl)-ethyl]-3-methylsulfanyl-5,6,7,8-tetrahydro-imidazo[1,5-a]pyrazine. LC-MS: t_{R}=0.84 min.; [M+H]⁺: 378.06 g/mol.

Subsequent protection of the secondary amine, and purification by FC (AcOEt/heptane=1/5 to AcOEt) allowed the isolation of 8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester as a pale yellow solid (1.250 g; 80% for two steps). LC-MS: t_{R}=0.98 min.; [M+H]⁺: 478.19 g/mol.

8-[2-(4-difluoromethoxy-phenyl)-ethyl]-3-ethyl-1-iodo-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 7.0 bars; 10 min.) between 2-(2-ethyl-4-iodo-imidazol-1-yl)-ethylamine (11.30 mmol) and 3-(4-difluoromethoxy-phenyl)-propionaldehyde (3.528 g; 17.62 mmol) afforded 8-[2-(4-difluoromethoxy-phenyl)-ethyl]-3-ethyl-1-iodo-5,6,7,8-tetrahydro-imidazo[1,5-a]pyrazine. LC-MS: t_{R}=0.82 min.; [M+H]⁺: 447.87 g/mol.

Subsequent protection of the secondary amine, and purification by FC (DCM/MeOH=25/1) allowed the isolation of 8-[2-(4-difluoromethoxy-phenyl)-ethyl]-3-ethyl-1-iodo-5,6-dihydro-8H-imidazo[1,5-a]pyrazine-7-carboxylic acid tert-butyl ester as a pale yellow oil (4.230 g; 68% for two steps). LC-MS: t_{R}=0.94 min.; [M+H]⁺: 548.41 g/mol.

8-[2-(4-difluoromethoxy-3-fluoro-phenyl)-ethyl]-3-ethyl-1-iodo-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 7.0 bars; 10 min.) between 2-(2-ethyl-4-iodo-imidazol-1-yl)-ethylamine (11.62 mmol) and 3-(4-difluoromethoxy-3-fluoro-phenyl)-propionaldehyde (3.042 g; 13.94 mmol) afforded 8-[2-(4-difluoromethoxy-3-fluoro-phenyl)-ethyl]-3-ethyl-1-iodo-5,6,7,8-tetrahydro-imidazo[1,5-a]pyrazine. LC-MS: t_{R}=0.83 min.; [M+H]⁺: 466.02 g/mol.

Subsequent protection of the secondary amine, and purification by FC (AcOEt/heptane=1/10 to 9/10) allowed the isolation of 8-[2-(4-difluoromethoxy-3-fluoro-phenyl)-ethyl]-3-ethyl-1-iodo-5,6-dihydro-8H-imidazo[1,5-a]pyrazine-7-carboxylic acid tert-butyl ester as a yellow oil (6.540 g; 99% for two steps). LC-MS: t_{R}=0.97 min.; [M+H]⁺: 566.32 g/mol.

8-[2-(3-difluoromethoxy-phenyl)-ethyl]-3-ethyl-1-iodo-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 7.0 bars; 10 min.) between 2-(2-ethyl-4-iodo-imidazol-1-yl)-ethylamine (4.43 mmol) and 3-(3-difluoromethoxy-phenyl)-propionaldehyde (888 mg; 4.43 mmol) afforded 8-[2-(3-difluoromethoxy-phenyl)-ethyl]-3-ethyl-1-iodo-5,6,7,8-tetrahydro-imidazo[1,5-a]pyrazine. LC-MS: t_{R}=0.74 min.; [M+H]⁺: 447.96 g/mol.

Subsequent protection of the secondary amine, and purification by FC (AcOEt/heptane=1/1 to 7/3) allowed the isolation of 8-[2-(3-difluoromethoxy-phenyl)-ethyl]-3-ethyl-1-iodo-5,6-dihydro-8H-imidazo[1,5-a]pyrazine-7-carboxylic acid tert-butyl ester as a pale yellow oil (1.320 g; 54% for two steps). LC-MS: t_{R}=0.95 min.; [M+H]⁺: 548.45 g/mol.

8-[2-(4-difluoromethoxy-phenyl)-ethyl]-1-iodo-3-methyl-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 6.0 bars; 10 min.) between 2-(4-iodo-2-methyl-imidazol-1-yl)-ethylamine (5.66 mmol) and 3-(4-difluoromethoxy-phenyl)-propionaldehyde (1.359 g; 6.79 mmol) afforded 8-[2-(4-difluoromethoxy-phenyl)-ethyl]-1-iodo-3-methyl-5,6,7,8-tetrahydro-imidazo[1,5-a]pyrazine. LC-MS (basic conditions): t_{R}=0.79 min.; [M+H]⁺: 433.99 g/mol.

Subsequent protection of the secondary amine, and purification by FC (AcOEt/heptane=1/5 to AcOEt) allowed the isolation of 8-[2-(4-difluoromethoxy-phenyl)-ethyl]-1-iodo-3-methyl-5,6-dihydro-8H-imidazo[1,5-a]pyrazine-7-carboxylic acid tert-butyl ester as a pale yellow solid (2.430 g; 80% for two steps). LC-MS: t_{R}=0.94 min.; [M+H]⁺: 534.38 g/mol.

1-methyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 7.0 bars; 10 min.) between 2-(4-methyl-imidazol-1-yl)-ethylamine (4.62 mmol) and 3-(4-trifluoromethyl-phenyl)-propionaldehyde (934 mg; 4.62 mmol) afforded 1-methyl-8-[2-(4-trifluoromethyl-phenyl)-ethyl]-5,6,7,8-tetrahydro-imidazo[1,5-a]pyrazine. LC-MS: t_{R}=0.69 min.; [M+H]⁺: 310.36 g/mol.

Subsequent protection of the secondary amine, and purification by FC (DCM/MeOH=20/1) allowed the isolation of 1-methyl-8-[2-(4-trifluoromethyl-phenyl)-ethyl]-5,6-dihydro-8H-imidazo[1,5-a]pyrazine-7-carboxylic acid tert-butyl ester as a beige solid (675 mg; 36% for two steps). LC-MS: t_{R}=0.92 min.; [M+H]⁺: 410.60 g/mol.

3-ethyl-1-iodo-8-[2-(4-trifluoromethyl-phenyl)-ethyl]-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 6.0 bars; 10 min.) between 2-(2-ethyl-4-iodo-imidazol-1-yl)-ethylamine (12.60 mmol) and 3-(4-trifluoromethyl-phenyl)-propionaldehyde (2.548 g; 12.60 mmol) afforded 3-ethyl-1-iodo-8-[2-(4-trifluoromethyl-phenyl)-ethyl]-5,6,7,8-tetrahydro-imidazo[1,5-a]pyrazine. LC-MS: t_{R}=0.77 min.; [M+H]⁺: 449.74 g/mol.

Subsequent protection of the secondary amine, and purification by FC (AcOEt/heptane=1/10 to 7/10) allowed the isolation of 3-ethyl-1-iodo-8-[2-(4-trifluoromethyl-phenyl)-ethyl]-5,6-dihydro-8H-imidazo[1,5-a]pyrazine-7-carboxylic acid tert-butyl ester as a pale yellow solid (3.490 g; 50% for two steps). LC-MS: t_{R}=0.98 min.; [M+H]⁺: 549.87 g/mol.

3-cyclopropylmethyl-1-iodo-8-[2-(4-trifluoromethyl-phenyl)-ethyl]-5,6-dihydro-8H-imidazo[1,5-a]pyrazine-7-carboxylic acid tert-butyl ester

According to the described general procedure (GP14), microwave-assisted Pictet-Spengler reaction (60 W; 140° C.; 6.0 bars; 10 min.) between 2-(2-cyclopropylmethyl-4-iodo-imidazol-1-yl)-ethylamine (6.31 mmol) and 3-(4-trifluoromethyl-phenyl)-propionaldehyde (1.276 g; 6.31 mmol) afforded 3-cyclopropylmethyl-1-iodo-8-[2-(4-trifluoromethyl-phenyl)-ethyl]-5,6,7,8-tetrahydro-imidazo[1,5-a]pyrazine. LC-MS: t_{R}=0.80 min.; [M+H]⁺: 476.23 g/mol.

Subsequent protection of the secondary amine, and purification by FC (DCM/MeOH=80/1) allowed the isolation of 3-cyclopropylmethyl-1-iodo-8-[2-(4-trifluoromethyl-phenyl)-ethyl]-5,6-dihydro-8H-imidazo[1,5-a]pyrazine-7-carboxylic acid tert-butyl ester as a yellow solid (2.120 g; 58% for two steps). LC-MS: t_{R}=1.00 min.; [M+H]⁺: 575.93 g/mol.

3-cyclopropyl-1-iodo-8-[2-(4-trifluoromethyl-phenyl)-ethyl]-5,6-dihydro-8H-imidazo[1,5-a]pyrazine-7-carboxylic acid tert-butyl ester

According to the general procedure (GP14), microwave-assisted Pictet-Spengler reaction (70 W; 120° C.; 6.0 bars; 10 min.) between 2-(2-cyclopropyl-4-iodo-imidazol-1-yl)-ethylamine (26.75 mmol) and 3-(4-trifluoromethyl-phenyl)-propionaldehyde (6.489 g; 32.10 mmol) afforded 3-cyclopropyl-1-iodo-8-[2-(4-trifluoromethyl-phenyl)-ethyl]-5,6,7,8-tetrahydro-imidazo[1,5-a]pyrazine as a yellow oil. LC-MS (basic conditions): t_{R}=0.90 min.; [M+H]⁺: 461.90 g/mol.

Subsequent protection of the secondary amine, and purification by FC (DCM/MeOH=25/1) allowed the isolation of 3-cyclopropyl-1-iodo-8-[2-(4-trifluoromethyl-phenyl)-ethyl]-5,6-dihydro-8H-imidazo[1,5-a]pyrazine-7-carboxylic acid tert-butyl ester as a pale yellow solid (11.440 g; 76%). LC-MS: t_{R}=1.00 min.; [M+H]⁺: 561.94 g/mol.

3-cyclopropyl-1-iodo-8-[2-(3-trifluoromethyl-phenyl)-ethyl]-5,6-dihydro-8H-imidazo[1,5-a]pyrazine-7-carboxylic acid tert-butyl ester

According to the general procedure (GP14), microwave-assisted Pictet-Spengler reaction (70 W; 140° C.; 7.0 bars; 10 min.) between 2-(2-cyclopropyl-4-iodo-imidazol-1-yl)-ethylamine (9.70 mmol) and 3-(3-trifluoromethyl-phenyl)-propionaldehyde (1.961 g; 9.70 mmol) afforded 3-cyclopropyl-1-iodo-8-[2-(3-trifluoromethyl-phenyl)-ethyl]-5,6,7,8-tetrahydro-imidazo[1,5-a]pyrazine as a brown oil. LC-MS: t_{R}=0.79 min.; [M+H]⁺: 461.97 g/mol.

Subsequent protection of the secondary amine, and purification by FC (AcOEt/heptane=1/9 then AcOEt) allowed the isolation of 3-cyclopropyl-1-iodo-8-[2-(3-trifluoromethyl-phenyl)-ethyl]-5,6-dihydro-8H-imidazo[1,5-a]pyrazine-7-carboxylic acid tert-butyl ester as a brown oil (4.360 g; 80%). LC-MS: t_{R}=1.00 min.; [M+H]⁺: 562.30 g/mol.

3-cyclopropyl-8-[2-(2-fluoro-4-trifluoromethyl-phenyl)-ethyl]-1-iodo-5,6-dihydro-8H-imidazo[1,5-a]pyrazine-7-carboxylic acid tert-butyl ester

According to the general procedure (GP14), microwave-assisted Pictet-Spengler reaction (70 W; 140° C.; 7.0 bars; 10 min.) between 2-(2-cyclopropyl-4-iodo-imidazol-1-yl)-ethylamine (10.10 mmol) and 3-(2-fluoro-4-trifluoromethyl-phenyl)-propionaldehyde (2.223 g; 10.10 mmol) afforded 3-cyclopropyl-8-[2-(2-fluoro-4-trifluoromethyl-phenyl)-ethyl]-1-iodo-5,6,7,8-tetrahydro-imidazo[1,5-a]pyrazine as a brown oil. LC-MS: t_{R}=0.81 min.; [M+H]⁺: 479.74 g/mol.

Subsequent protection of the secondary amine, and purification by FC (AcOEt/heptane=1/9 then AcOEt) allowed the isolation of 3-cyclopropyl-8-[2-(2-fluoro-4-trifluoromethyl-phenyl)-ethyl]-1-iodo-5,6-dihydro-8H-imidazo[1,5-a]pyrazine-7-carboxylic acid tert-butyl ester as a brown oil (5.510 g; 94%). LC-MS: t_{R}=1.00 min.; [M+H]⁺: 580.27 g/mol.

1-iodo-8-[2-(4-trifluoromethyl-phenyl)-ethyl]-5,6-dihydro-8H-imidazo[1,5-a]pyrazine-7-carboxylic acid tert-butyl ester

According to the general procedure (GP14), microwave-assisted Pictet-Spengler reaction (70 W; 140° C.; 6.0 bars; 10 min.) between 2-(4-iodo-imidazol-1-yl)-ethylamine (25.42 mmol) and 3-(4-trifluoromethyl-phenyl)-propionaldehyde (7.144 g; 35.33 mmol) afforded 1-iodo-8-[2-(4-trifluoromethyl-phenyl)-ethyl]-5,6,7,8-tetrahydro-imidazo[1,5-a]pyrazine as a brown oil. LC-MS: t_{R}=0.79 min.; [M+H]⁺: 421.88 g/mol.

Subsequent protection of the secondary amine, and purification by FC (DCM/MeOH=25/1) allowed the isolation of 1-iodo-8-[2-(4-trifluoromethyl-phenyl)-ethyl]-5,6-dihydro-8H-imidazo[1,5-a]pyrazine-7-carboxylic acid tert-butyl ester as a brown oil (8.660 g; 65%). LC-MS: t_{R}=0.99 min.; [M+H]⁺: 521.85 g/mol.

1-ethyl-8-[2-(3-fluoro-4-trifluoromethyl-phenyl)-ethyl]-5,6-dihydro-8H-imidazo[1,5-a]pyrazine-7-carboxylic acid tert-butyl ester

According to the general procedure (GP14), microwave-assisted Pictet-Spengler reaction (60 W; 160° C.; 10.0 bars; 40 min.) between 2-(4-ethyl-imidazol-1-yl)-ethylamine (8.34 mmol) and 3-(3-fluoro-4-trifluoromethyl-phenyl)-propionaldehyde (1.837 g; 8.34 mmol) afforded 1-ethyl-8-[2-(3-fluoro-4-trifluoromethyl-phenyl)-ethyl]-5,6,7,8-tetrahydro-imidazo[1,5-a]pyrazine as an orange oil. LC-MS: t_{R}=0.71 min.; [M+H]⁺: 342.39 g/mol.

Subsequent protection of the secondary amine, and purification by FC (DCM/MeOH=19/1) allowed the isolation of 1-ethyl-8-[2-(3-fluoro-4-trifluoromethyl-phenyl)-ethyl]-5,6-dihydro-8H-imidazo[1,5-a]pyrazine-7-carboxylic acid tert-butyl ester as a yellow oil (1.900 g; 52%). LC-MS: t_{R}=0.94 min.; [M+H]⁺: 442.47 g/mol.

### D. Functionalization and Derivatization of 5,6,7,8-tetrahydro-imidazo[1,5-a]pyrazine Derivatives

D.1 Chlorination of 5,6,7,8-tetrahydro-imidazo[1,5-a]pyrazine Derivatives

### D1.1 First General Procedure for Chlorination of the Imidazole Ring

1-chloro-3-cyclopropyl-8-[2-(3-fluoro-4-trifluoromethyl-phenyl)-ethyl]-5,6-dihydro-8H-imidazo[1,5-a]pyrazine-7-carboxylic acid tert-butyl ester

### [General Procedure for Chlorination of the Imidazole Ring (GP15A)]

A cooled (−78° C.) solution of 3-cyclopropyl-8-[2-(3-fluoro-4-trifluoromethyl-phenyl)-ethyl]-1-iodo-5,6-dihydro-8H-imidazo[1,5-a]pyrazine-7-carboxylic acid tert-butyl ester (1.000 g; 1.72 mmol) in anh. THF (15 ml) was treated dropwise with a solution of 1.6 M n-BuLi in hexanes (1.44 ml; 2.31 mmol). The resulting solution was additionally stirred at −78° C. for 10 min., and was then treated dropwise with a solution of hexachloroethane (1.634 g; 6.90 mmol; 4.0 eq.) in anh. THF (5 ml). The reaction mixture was further stirred at −78° C. for 30 min. The mixture was then quenched with water (1 ml), diluted with Et₂O (50 ml), and was allowed to warm-up to rt. The organic layer was washed with water (80 ml), dried over anh. MgSO₄, filtered, and concentrated to dryness under reduced pressure. The crude was purified by FC (ethyl acetate/heptane=3/2) to give 1-chloro-3-cyclopropyl-8-[2-(3-fluoro-4-trifluoromethyl-phenyl)-ethyl]-5,6-dihydro-8H-imidazo[1,5-a]pyrazine-7-carboxylic acid tert-butyl ester as a pale yellow solid (491 mg; 58%). LC-MS: t_{R}=1.06 min.; [M+H]⁺: 488.39 g/mol.

### D.1.2 Second General Procedure for Chlorination of the Imidazole Ring

1-chloro-3-cyclopropyl-8-[2-(4-fluoro-3-trifluoromethyl-phenyl)-ethyl]-5,6-dihydro-8H-imidazo[1,5-a]pyrazine-7-carboxylic acid tert-butyl ester

### [General Procedure for the Chlorination of the Imidazole Ring (GP15B)]

A mixture of 3-cyclopropyl-8-[2-(4-fluoro-3-trifluoromethyl-phenyl)-ethyl]-1-iodo-5,6-dihydro-8H-imidazo[1,5-a]pyrazine-7-carboxylic acid tert-butyl ester (1.420 g; 2.45 mmol; 1.0 eq.), 10% palladium on activated charcoal (213 mg; 15% in mass), and anh. K₂CO₃ (847 mg; 6.12 mmol; 2.5 eq.) in anh. MeOH (25 ml) was stirred at rt, under hydrogen atmosphere (1 atm), for 45 min. Filtration over a pad of celite, and subsequent concentration to dryness afforded a crude heterogeneous residue which was dissolved in DCM (50 ml), and water (50 ml). The organic layer was then dried over anh. MgSO₄, filtered, and concentrated to dryness under reduced pressure to give 3-cyclopropyl-8-[2-(4-fluoro-3-trifluoromethyl-phenyl)-ethyl]-5,6-dihydro-8H-imidazo[1,5-a]pyrazine-7-carboxylic acid tert-butyl ester as a colorless oil (1.073 g; 97%). LC-MS: t_{R}=0.97 min.; [M+H]⁺: 454.34 g/mol.

To a solution of 3-cyclopropyl-8-[2-(4-fluoro-3-trifluoromethyl-phenyl)-ethyl]-5,6-dihydro-8H-imidazo[1,5-a]pyrazine-7-carboxylic acid tert-butyl ester (1.073 g; 2.36 mmol; 1.0 eq.) in anh. MeCN (20 ml) was added dropwise, at rt, a solution of NCS (322 mg; 2.36 mmol; 1.0 eq.) in anh. MeCN (10 ml). The resulting solution was then heated to 90° C., under nitrogen, for 2 h 30. Concentration to dryness under reduced pressure afforded an oily residue which was dissolved in AcOEt (100 ml), and this organic layer was successively washed with aq. saturated NaHCO₃ (2×50 ml), and brine (50 ml). The resulting organic layer was then dried over anh. MgSO₄, filtered, and concentrated to dryness under reduced pressure. Purification by FC (DCM/MeOH=20/1) afforded 1-chloro-3-cyclopropyl-8-[2-(4-fluoro-3-trifluoromethyl-phenyl)-ethyl]-5,6-dihydro-8H-imidazo[1,5-a]pyrazine-7-carboxylic acid tert-butyl ester as a colorless oil (674 mg; 58%). LC-MS: t_{R}=1.04 min.; [M+H]⁺: 488.17 g/mol.

1-chloro-3-cyclopropyl-8-[2-(4-difluoromethoxy-phenyl)-ethyl]-5,6-dihydro-8H-imidazo[1,5-a]pyrazine-7-carboxylic acid tert-butyl ester

According to the described general procedure (GP15B), hydrogenation (rt; 1 h 45) of 3-cyclopropyl-8-[2-(4-difluoromethoxy-phenyl)-ethyl]-1-iodo-5,6-dihydro-8H-imidazo[1,5-a]pyrazine-7-carboxylic acid tert-butyl ester (1.100 g; 1.96 mmol) afforded 3-cyclopropyl-8-[2-(4-difluoromethoxy-phenyl)-ethyl]-5,6-dihydro-8H-imidazo[1,5-a]pyrazine-7-carboxylic acid tert-butyl ester as a slightly yellow oil (840 mg; 99%). LC-MS: t_{R}=0.92 min.; [M+H]⁺: 434.42 g/mol.

Subsequent chlorination (75° C.; 5 h) of 3-cyclopropyl-8-[2-(4-difluoromethoxy-phenyl)-ethyl]-5,6-dihydro-8H-imidazo[1,5-a]pyrazine-7-carboxylic acid tert-butyl ester (840 mg; 1.93 mmol), and purification by FC (DCM/MeOH=50/1) afforded 1-chloro-3-cyclopropyl-8-[2-(4-difluoromethoxy-phenyl)-ethyl]-5,6-dihydro-8H-imidazo[1,5-a]pyrazine-7-carboxylic acid tert-butyl ester as a yellow oil (451 mg; 50%). LC-MS: t_{R}=1.06 min.; [M+H]⁺: 468.31 g/mol.

1-chloro-3-cyclopropyl-8-[2-(3-fluoro-4-trifluoromethoxy-phenyl)-ethyl]-5,6-dihydro-8H-imidazo[1,5-a]pyrazine-7-carboxylic acid tert-butyl ester

According to the described general procedure (GP15B), hydrogenation (rt; 45 min.) of 3-cyclopropyl-8-[2-(3-fluoro-4-trifluoromethoxy-phenyl)-ethyl]-1-iodo-5,6-dihydro-8H-imidazo[1,5-a]pyrazine-7-carboxylic acid tert-butyl ester (1.790 g; 3.00 mmol) afforded 3-cyclopropyl-8-[2-(3-fluoro-4-trifluoromethoxy-phenyl)-ethyl]-5,6-dihydro-8H-imidazo[1,5-a]pyrazine-7-carboxylic acid tert-butyl ester as a pale yellow oil (1.370 g; 97%). LC-MS: t_{R}=0.98 min.; [M+H]⁺: 470.20 g/mol.

Subsequent chlorination (70° C.; 3 h 30) of 3-cyclopropyl-8-[2-(3-fluoro-4-trifluoromethoxy-phenyl)-ethyl]-5,6-dihydro-8H-imidazo[1,5-a]pyrazine-7-carboxylic acid tert-butyl ester (1.370 g; 2.91 mmol), and purification by FC (AcOEt/heptane=15/85 to 95/5) afforded 1-chloro-3-cyclopropyl-8-[2-(3-fluoro-4-trifluoromethoxy-phenyl)-ethyl]-5,6-dihydro-8H-imidazo[1,5-a]pyrazine-7-carboxylic acid tert-butyl ester as a yellow oil (1.020 g; 69%). LC-MS: t_{R}=1.12 min.; [M+H]⁺: 504.40 g/mol.

1-chloro-3-cyclopropyl-8-[2-(4-difluoromethoxy-3-fluoro-phenyl)-ethyl]-5,6-dihydro-8H-imidazo[1,5-a]pyrazine-7-carboxylic acid tert-butyl ester

According to the described general procedure (GP15B), hydrogenation (rt; 45 min.) of 3-cyclopropyl-8-[2-(4-difluoromethoxy-3-fluoro-phenyl)-ethyl]-1-iodo-5,6-dihydro-8H-imidazo[1,5-a]pyrazine-7-carboxylic acid tert-butyl ester (1.400 g; 2.42 mmol) afforded 3-cyclopropyl-8-[2-(4-difluoromethoxy-3-fluoro-phenyl)-ethyl]-5,6-dihydro-8H-imidazo[1,5-a]pyrazine-7-carboxylic acid tert-butyl ester as a colorless oil (1.050 g; 96%). LC-MS (basic conditions): t_{R}=0.92 min.; [M+H]⁺: 452.19 g/mol.

Subsequent chlorination (70° C.; 3.5 h) of 3-cyclopropyl-8-[2-(4-difluoromethoxy-3-fluoro-phenyl)-ethyl]-5,6-dihydro-8H-imidazo[1,5-a]pyrazine-7-carboxylic acid tert-butyl ester (1.078 g; 2.38 mmol), and purification by FC (AcOEt/heptane=1/4 to 3/2) afforded 1-chloro-3-cyclopropyl-8-[2-(4-difluoromethoxy-3-fluoro-phenyl)-ethyl]-5,6-dihydro-8H-imidazo[1,5-a]pyrazine-7-carboxylic acid tert-butyl ester as a colorless solid (558 mg; 48%). LC-MS: t_{R}=1.08 min.; [M+H]⁺: 486.44 g/mol.

1-chloro-3-methylsulfanyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP15B), chlorination (70° C. for 3.5 h; and then 77° C. for 6.5 h) of 3-methylsulfanyl-8-[2-(4-trifluoromethyl-phenyl)-ethyl]-5,6-dihydro-8H-imidazo[1,5-a]pyrazine-7-carboxylic acid tert-butyl ester (529 mg; 1.19 mmol), and purification by FC (DCM/MeOH=50/1) afforded 1-chloro-3-methylsulfanyl-8-[2-(4-trifluoromethyl-phenyl)-ethyl]-5,6-dihydro-8H-imidazo[1,5-a]pyrazine-7-carboxylic acid tert-butyl ester as a slightly beige solid (346 mg; 61%). LC-MS: t_{R}=1.18 min.; [M+H]⁺: 476.28 g/mol.

1-chloro-8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester

According to the described general procedure (GP15B), chlorination (70° C.; 3.5 h) of 8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester (1.250 g; 2.61 mmol), and purification by FC (AcOEt/heptane=1/10 to AcOEt) afforded 1-chloro-8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester as a yellow solid (970 mg; 72%). LC-MS: t_{R}=1.19 min.; [M+H]⁺: 511.46 g/mol.

1-chloro-8-[2-(4-difluoromethoxy-phenyl)-ethyl]-3-ethyl-5,6-dihydro-8H-imidazo[1,5-a]pyrazine-7-carboxylic acid tert-butyl ester

According to the described general procedure (GP15B), hydrogenation (rt; 1 h) of 8-[2-(4-difluoromethoxy-phenyl)-ethyl]-3-ethyl-1-iodo-5,6-dihydro-8H-imidazo[1,5-a]pyrazine-7-carboxylic acid tert-butyl ester (4.230 g; 7.72 mmol) afforded 8-[2-(4-difluoromethoxy-phenyl)-ethyl]-3-ethyl-5,6-dihydro-8H-imidazo[1,5-a]pyrazine-7-carboxylic acid tert-butyl ester as a yellow oil (3.060 g; 94%). LC-MS: t_{R}=0.91 min.; [M+H]⁺: 422.40 g/mol.

Subsequent chlorination (70° C.; 3.5 h) of 8-[2-(4-difluoromethoxy-phenyl)-ethyl]-3-ethyl-5,6-dihydro-8H-imidazo[1,5-a]pyrazine-7-carboxylic acid tert-butyl ester (3.060 g; 7.26 mmol), and purification by FC (AcOEt/heptane=1/10 to 3/5) afforded 1-chloro-8-[2-(4-difluoromethoxy-phenyl)-ethyl]-3-ethyl-5,6-dihydro-8H-imidazo[1,5-a]pyrazine-7-carboxylic acid tert-butyl ester as a yellow oil (2.070 g; 63%). LC-MS (basic conditions): t_{R}=0.98 min.; [M+H]⁺: 456.19 g/mol.

1-chloro-8-[2-(4-difluoromethoxy-3-fluoro-phenyl)-ethyl]-3-ethyl-5,6-dihydro-8H-imidazo[1,5-a]pyrazine-7-carboxylic acid tert-butyl ester

According to the described general procedure (GP15B), hydrogenation (rt; 45 min.) of 8-[2-(4-difluoromethoxy-3-fluoro-phenyl)-ethyl]-3-ethyl-1-iodo-5,6-dihydro-8H-imidazo[1,5-a]pyrazine-7-carboxylic acid tert-butyl ester (6.540 g; 11.56 mmol) afforded 8-[2-(4-difluoromethoxy-3-fluoro-phenyl)-ethyl]-3-ethyl-5,6-dihydro-8H-imidazo[1,5-a]pyrazine-7-carboxylic acid tert-butyl ester as a yellow oil (4.950 g; 97%). LC-MS: t_{R}=0.93 min.; [M+H]⁺: 440.36 g/mol.

Subsequent chlorination (70° C.; 3.5 h) of 8-[2-(4-difluoromethoxy-3-fluoro-phenyl)-ethyl]-3-ethyl-5,6-dihydro-8H-imidazo[1,5-a]pyrazine-7-carboxylic acid tert-butyl ester (4.950 g; 11.26 mmol), and purification by FC (AcOEt/heptane=1/10 to 3/5) afforded 1-chloro-8-[2-(4-difluoromethoxy-3-fluoro-phenyl)-ethyl]-3-ethyl-5,6-dihydro-8H-imidazo[1,5-a]pyrazine-7-carboxylic acid tert-butyl ester as a yellow oil (3.100 g; 58%). LC-MS: t_{R}=1.05 min.; [M+H]⁺: 474.24 g/mol.

1-chloro-8-[2-(3-difluoromethoxy-phenyl)-ethyl]-3-ethyl-5,6-dihydro-8H-imidazo[1,5-a]pyrazine-7-carboxylic acid tert-butyl ester

According to the described general procedure (GP15B), hydrogenation (rt; 1 h 15) of 8-[2-(3-difluoromethoxy-phenyl)-ethyl]-3-ethyl-1-iodo-5,6-dihydro-8H-imidazo[1,5-a]pyrazine-7-carboxylic acid tert-butyl ester (1.320 g; 2.41 mmol) afforded 8-[2-(3-difluoromethoxy-phenyl)-ethyl]-3-ethyl-5,6-dihydro-8H-imidazo[1,5-a]pyrazine-7-carboxylic acid tert-butyl ester as a yellow oil (910 mg; 89%). LC-MS: t_{R}=0.91 min.; [M+H]⁺: 422.50 g/mol.

Subsequent chlorination (70° C.; 3.5 h) of 8-[2-(3-difluoromethoxy-phenyl)-ethyl]-3-ethyl-5,6-dihydro-8H-imidazo[1,5-a]pyrazine-7-carboxylic acid tert-butyl ester (910 mg; 2.15 mmol), and purification by FC (AcOEt/heptane=1/1 to 7/3) afforded 1-chloro-8-[2-(3-difluoromethoxy-phenyl)-ethyl]-3-ethyl-5,6-dihydro-8H-imidazo[1,5-a]pyrazine-7-carboxylic acid tert-butyl ester as a yellow oil (580 mg; 59%). LC-MS: t_{R}=1.04 min.; [M+H]⁺: 456.34 g/mol.

1-chloro-8-[2-(4-difluoromethoxy-phenyl)-ethyl]-3-methyl-5,6-dihydro-8H-imidazo[1,5-a]pyrazine-7-carboxylic acid tert-butyl ester

According to the described general procedure (GP15B), hydrogenation (rt; 45 min.) of 8-[2-(4-difluoromethoxy-phenyl)-ethyl]-1-iodo-3-methyl-5,6-dihydro-8H-imidazo[1,5-a]pyrazine-7-carboxylic acid tert-butyl ester (2.430 g; 4.55 mmol) afforded 8-[2-(4-difluoromethoxy-phenyl)-ethyl]-3-methyl-5,6-dihydro-8H-imidazo[1,5-a]pyrazine-7-carboxylic acid tert-butyl ester as a pale yellow oil (1.760 g; 95%). LC-MS: t_{R}=0.91 min.; [M+H]⁺: 408.36 g/mol.

Subsequent chlorination (70° C.; 3.5 h) of 8-[2-(4-difluoromethoxy-phenyl)-ethyl]-3-methyl-5,6-dihydro-8H-imidazo[1,5-a]pyrazine-7-carboxylic acid tert-butyl ester (1.760 g; 4.31 mmol), and purification by FC (AcOEt/heptane=1/4 to 3/2) afforded 1-chloro-8-[2-(4-difluoromethoxy-phenyl)-ethyl]-3-methyl-5,6-dihydro-8H-imidazo[1,5-a]pyrazine-7-carboxylic acid tert-butyl ester as a colorless solid (1.100 g; 58%). LC-MS (basic conditions): t_{R}=0.94 min.; [M+H]⁺: 442.11 g/mol.

1-chloro-3-cyclopropylmethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP15B), hydrogenation (rt; 1.5 h) of 3-cyclopropyl methyl-1-iodo-8-[2-(4-trifluoromethyl-phenyl)-ethyl]-5,6-dihydro-8H-imidazo[1,5-a]pyrazine-7-carboxylic acid tert-butyl ester (1.000 g; 1.73 mmol) afforded 3-cyclopropylmethyl-8-[2-(4-trifluoromethyl-phenyl)-ethyl]-5,6-dihydro-8H-imidazo[1,5-a]pyrazine-7-carboxylic acid tert-butyl ester as a yellow oil (730 mg; 93%). LC-MS: t_{R}=0.96 min.; [M+H]⁺: 450.22 g/mol.

Subsequent chlorination (70° C.; 1 h 50) of 3-cyclopropylmethyl-8-[2-(4-trifluoromethyl-phenyl)-ethyl]-5,6-dihydro-8H-imidazo[1,5-a]pyrazine-7-carboxylic acid tert-butyl ester (730 mg; 1.62 mmol), and purification by FC (AcOEt/heptane=3/10 to 2/5) afforded 1-chloro-3-cyclopropylmethyl-8-[2-(4-trifluoromethyl-phenyl)-ethyl]-5,6-dihydro-8H-imidazo[1,5-a]pyrazine-7-carboxylic acid tert-butyl ester as a yellow oil (400 mg; 51%). LC-MS: t_{R}=1.05 min.; [M+H]⁺: 484.09 g/mol.

1-chloro-3-cyclopropyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP15B), hydrogenation (rt; 3.5 h) of 3-cyclopropyl-1-iodo-8-[2-(4-trifluoromethyl-phenyl)-ethyl]-5,6-dihydro-8H-imidazo[1,5-a]pyrazine-7-carboxylic acid tert-butyl ester (11.084 g; 19.74 mmol) afforded 3-cyclopropyl-8-[2-(4-trifluoromethyl-phenyl)-ethyl]-5,6-dihydro-8H-imidazo[1,5-a]pyrazine-7-carboxylic acid tert-butyl ester as a yellow solid (8.450 g; 98%). LC-MS: t_{R}=0.95 min.; [M+H]⁺: 436.18 g/mol.

Subsequent chlorination (70° C.; 4 h) of 3-cyclopropyl-8-[2-(4-trifluoromethyl-phenyl)-ethyl]-5,6-dihydro-8H-imidazo[1,5-a]pyrazine-7-carboxylic acid tert-butyl ester (8.450 g; 19.40 mmol), and purification by FC (AcOEt/heptane=1/9 then AcOEt) afforded 1-chloro-3-cyclopropyl-8-[2-(4-trifluoromethyl-phenyl)-ethyl]-5,6-dihydro-8H-imidazo[1,5-a]pyrazine-7-carboxylic acid tert-butyl ester as a yellow solid (7.730 g; 85%). LC-MS: t_{R}=1.10 min.; [M+H]⁺: 470.27 g/mol.

1-chloro-3-cyclopropyl-8-[2-(3-trifluoromethyl-phenyl)-ethyl]-5,6-dihydro-8H-imidazo[1,5-a]pyrazine-7-carboxylic acid tert-butyl ester

According to the described general procedure (GP15B), hydrogenation (rt; 3 h) of 3-cyclopropyl-1-iodo-8-[2-(3-trifluoromethyl-phenyl)-ethyl]-5,6-dihydro-8H-imidazo[1,5-a]pyrazine-7-carboxylic acid tert-butyl ester (4.360 g; 7.76 mmol) afforded 3-cyclopropyl-8-[2-(3-trifluoromethyl-phenyl)-ethyl]-5,6-dihydro-8H-imidazo[1,5-a]pyrazine-7-carboxylic acid tert-butyl ester as a yellow oil (3.170 g; 94%). LC-MS: t_{R}=0.97 min.; [M+H]⁺: 435.87 g/mol.

Subsequent chlorination (70° C.; 4 h) of 3-cyclopropyl-8-[2-(3-trifluoromethyl-phenyl)-ethyl]-5,6-dihydro-8H-imidazo[1,5-a]pyrazine-7-carboxylic acid tert-butyl ester (3.170 g; 7.27 mmol), and purification by FC (AcOEt/heptane=1/9 then AcOEt) afforded 1-chloro-3-cyclopropyl-8-[2-(3-trifluoromethyl-phenyl)-ethyl]-5,6-dihydro-8H-imidazo[1,5-a]pyrazine-7-carboxylic acid tert-butyl ester as a yellow solid (2.150 g; 63%). LC-MS: t_{R}=1.09 min.; [M+H]⁺: 469.99 g/mol.

1-chloro-3-cyclopropyl-8-[2-(2-fluoro-4-trifluoromethyl-phenyl)-ethyl]-5,6-dihydro-8H-imidazo[1,5-a]pyrazine-7-carboxylic acid tert-butyl ester

According to the described general procedure (GP15B), hydrogenation (rt; 1 h 45) of 3-cyclopropyl-8-[2-(2-fluoro-4-trifluoromethyl-phenyl)-ethyl]-1-iodo-5,6-dihydro-8H-imidazo[1,5-a]pyrazine-7-carboxylic acid tert-butyl ester (5.510 g; 9.51 mmol) afforded 3-cyclopropyl-8-[2-(2-fluoro-4-trifluoromethyl-phenyl)-ethyl]-5,6-dihydro-8H-imidazo[1,5-a]pyrazine-7-carboxylic acid tert-butyl ester as a yellow oil (3.840 g; 89%). LC-MS: t_{R}=0.97 min.; [M+H]⁺: 454.10 g/mol.

Subsequent chlorination (70° C.; 4 h) of 3-cyclopropyl-8-[2-(2-fluoro-4-trifluoromethyl-phenyl)-ethyl]-5,6-dihydro-8H-imidazo[1,5-a]pyrazine-7-carboxylic acid tert-butyl ester (3.840 g; 8.46 mmol), and purification by FC (AcOEt/heptane=1/9 then AcOEt) afforded 1-chloro-3-cyclopropyl-8-[2-(2-fluoro-4-trifluoromethyl-phenyl)-ethyl]-5,6-dihydro-8H-imidazo[1,5-a]pyrazine-7-carboxylic acid tert-butyl ester as a yellow solid (2.810 g; 68%). LC-MS: t_{R}=1.11 min.; [M+H]⁺: 488.26 g/mol.

### D.1.3 Third General Procedure for the Chlorination of the Imidazole Ring

1-chloro-8-[2-(4-chloro-3-fluoro-phenyl)-ethyl]-3-cyclopropyl-5,6-dihydro-8H-imidazo[1,5-a]pyrazine-7-carboxylic acid tert-butyl ester

### [General Procedure for the Chlorination of the Imidazole Ring (GP15C)]

A cooled (−30° C.) solution of 8-[2-(4-chloro-3-fluoro-phenyl)-ethyl]-3-cyclopropyl-1-iodo-5,6-dihydro-8H-imidazo[1,5-a]pyrazine-7-carboxylic acid tert-butyl ester (1.050 g; 1.92 mmol) in anh. THF (50 ml) was treated dropwise with a solution of 1M EtMgBr in THF (30.8 ml; 30.8 mmol). After completion of the addition, the reaction mixture was allowed to warm-up to 0° C., and stirring at this temperature was continued until complete removal of the iodine substituent. The mixture was again cooled to −30° C., treated with water (35 ml), diluted with Et₂O (100 ml), and was allowed to warm-up to rt. This solution was washed with brine (2×150 ml), and the organic layer was dried over anh. MgSO₄, filtered, and concentrated to dryness under reduced pressure. The crude was purified by FC (DCM/MeOH=95/5) to afford 8-[2-(4-chloro-3-fluoro-phenyl)-ethyl]-3-cyclopropyl-5,6-dihydro-8H-imidazo[1,5-a]pyrazine-7-carboxylic acid tert-butyl ester as a yellow solid (790 mg; 98%). LC-MS: t_{R}=0.93 min.; [M+H]⁺: 420.37 g/mol.

To a solution of 8-[2-(4-chloro-3-fluoro-phenyl)-ethyl]-3-cyclopropyl-5,6-dihydro-8H-imidazo[1,5-a]pyrazine-7-carboxylic acid tert-butyl ester (780 mg; 1.85 mmol) in anh. MeCN (20 ml) was added dropwise, at rt, a solution of NCS (253 mg; 1.85 mmol; 1.0 eq.) in anh. MeCN (10 ml). The resulting solution was then heated to 90° C., under nitrogen, for 2 h 30. Concentration to dryness under reduced pressure afforded an oily residue which was dissolved in AcOEt (100 ml), and this organic layer was successively washed with aq. saturated NaHCO₃ (2×50 ml), and brine (50 ml). The resulting organic layer was then dried over anh. MgSO₄, filtered, and concentrated to dryness under reduced pressure. Purification by FC (AcOEt/heptane=4/6) afforded 1-chloro-8-[2-(4-chloro-3-fluoro-phenyl)-ethyl]-3-cyclopropyl-5,6-dihydro-8H-imidazo[1,5-a]pyrazine-7-carboxylic acid tert-butyl ester as a yellow oil (680 mg; 81%). LC-MS: t_{R}=1.09 min.; [M+H]⁺: 454.28 g/mol.

D.2 Bromination of 5,6,7,8-tetrahydro-imidazo[1,5-a]pyrazine Derivatives
3-bromo-1-methyl-8-[2-(4-trifluoromethyl-phenyl)-ethyl]-5,6-dihydro-8H-imidazo[1,5-a]pyrazine-7-carboxylic acid tert-butyl ester
[General Procedure for Bromination of 5,6,7,8-tetrahydro-imidazo[1,5-a]pyrazine Derivatives (GP16)]

To a solution of 1-methyl-8-[2-(4-trifluoromethyl-phenyl)-ethyl]-5,6-dihydro-8H-imidazo[1,5-a]pyrazine-7-carboxylic acid tert-butyl ester (675 mg; 1.64 mmol; 1.0 eq.) in anh. MeCN (27 ml) was added dropwise, at rt, a solution of NBS (294 mg; 1.64 mmol; 1.0 eq.) in anh. MeCN (14 ml). The resulting yellow solution was then further stirred at rt, under nitrogen, for 45 min. Concentration to dryness under reduced pressure afforded an oily residue which was dissolved in AcOEt (100 ml), and this organic layer was successively washed with aq. saturated NaHCO₃ (2×30 ml), and brine (30 ml). The resulting yellow organic layer was then dried over anh. MgSO₄, filtered, and concentrated to dryness under reduced pressure. Purification by FC (DCM/MeOH=50/1) afforded the title product as a slightly beige solid (680 mg; 85%). LC-MS: t_{R}=1.04 min.; [M+H]⁺: 490.22 g/mol.

3-bromo-1-ethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP16), bromination (rt; 0.5 h) of 1-ethyl-8-[2-(4-trifluoromethyl-phenyl)-ethyl]-5,6-dihydro-8H-imidazo[1,5-a]pyrazine-7-carboxylic acid tert-butyl ester (2.530 g; 5.97 mmol) with NBS (1.169 g; 6.57 mmol; 1.1 eq.) and subsequent purification by FC (AcOEt/heptane=3/2) afforded 3-bromo-1-ethyl-8-[2-(4-trifluoromethyl-phenyl)-ethyl]-5,6-dihydro-8H-imidazo[1,5-a]pyrazine-7-carboxylic acid tert-butyl ester as a pale yellow solid (2.350 g; 78%). LC-MS: t_{R}=1.05 min.; [M+H]⁺: 502.21 g/mol.

3-bromo-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester

According to the described general procedure (GP16), bromination (rt; 1 h) of 1-ethyl-8-[2-(3-fluoro-4-trifluoromethyl-phenyl)-ethyl]-5,6-dihydro-8H-imidazo[1,5-a]pyrazine-7-carboxylic acid tert-butyl ester (1.000 g; 2.26 mmol) with NBS (0.403 g; 2.26 mmol; 1.0 eq.) and subsequent purification by FC (AcOEt/heptane=3/2) afforded 3-bromo-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester as a pale yellow solid (0.931 g; 79%). LC-MS: t_{R}=1.06 min.; [M+H]⁺: 520.30 g/mol.

D.3 Iodination of 5,6,7,8-tetrahydro-imidazo[1,5-a]pyrazine Derivatives
1-iodo-3-methylsulfanyl-8-[2-(4-trifluoromethyl-phenyl)-ethyl]-5,6-dihydro-8H-imidazo[1,5-a]pyrazine-7-carboxylic acid tert-butyl ester
[General Procedure for Iodination of 5,6,7,8-tetrahydro-imidazo[1,5-a]pyrazine Derivatives (GP17)]

To a solution of 3-methylsulfanyl-8-[2-(4-trifluoromethyl-phenyl)-ethyl]-5,6-dihydro-8H-imidazo[1,5-a]pyrazine-7-carboxylic acid tert-butyl ester (6.710 g; 15.19 mmol; 1.0 eq.) in anh. MeCN (200 ml) was added dropwise, at rt, a solution of N-iodosuccinimide (3.525 g; 15.19 mmol; 1.0 eq.) in anh. MeCN (50 ml). The resulting solution was then heated to 70° C., under nitrogen, for 2.5 h. Concentration to dryness under reduced pressure afforded an oily residue which was dissolved in AcOEt (400 ml), and this organic layer was successively washed with aq. saturated NaHCO₃ (2×300 ml), and brine (300 ml). The resulting organic layer was then dried over anh. MgSO₄, filtered, and concentrated to dryness under reduced pressure. Purification by FC (DCM/MeOH=50/1) afforded the title product as a yellow solid (6.880 g; 80%). LC-MS: t_{R}=1.14 min.; [M+H]⁺: 568.40 g/mol.

D.4 Trifluoromethylation of 5,6,7,8-tetrahydro-imidazo[1,5-a]pyrazine Derivatives

### D.4.1 First General Procedure for Trifluoromethylation of Imidazoles

1-methyl-3-trifluoromethyl-8-[2-(4-trifluoromethyl-phenyl)-ethyl]-5,6-dihydro-8H-imidazo[1,5-a]pyrazine-7-carboxylic acid tert-butyl ester
[General Procedure for Trifluoromethylation of 5,6,7,8-tetrahydro-imidazo[1,5-a]pyrazine Derivatives (GP18A)]

A flask was charged with 3-bromo-1-methyl-8-[2-(4-trifluoromethyl-phenyl)-ethyl]-5,6-dihydro-8H-imidazo[1,5-a]pyrazine-7-carboxylic acid tert-butyl ester (560 mg; 1.14 mmol; 1.0 eq.), anh. DMF (10 ml), anh. 1-methyl-2-pyrrolidinone (10 ml), copper(I) iodide CuI (655 mg; 3.44 mmol; 3.0 eq.), (trifluoromethyl)trimethylsilane (0.53 ml; 3.54 mmol; 3.0 eq.), and finally with KF (200 mg; 3.44 mmol; 3.0 eq.). The sealed heterogeneous mixture was then heated to 80° C. for 5.5 h. After cooling to rt, AcOEt (100 ml), toluene (50 ml), and water (100 ml) were successively added. After filtration, the orange organic layer was further washed with brine (50 ml), dried over anh. MgSO₄, filtered, and concentrated to dryness under reduced pressure. The resulting crude was purified by FC (DCM/MeOH=50/1] to give 1-methyl-3-trifluoromethyl-8-[2-(4-trifluoromethyl-phenyl)-ethyl]-5,6-dihydro-8H-imidazo[1,5-a]pyrazine-7-carboxylic acid tert-butyl ester as a beige solid which was further dried under HV (209 mg; 38%). LC-MS: t_{R}=1.16 min.; [M+H]⁺: 478.40 g/mol.

1-ethyl-3-trifluoromethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP18A), trifluoromethylation (80° C.; 5.5 h) of 3-bromo-1-ethyl-8-[2-(4-trifluoromethyl-phenyl)-ethyl]-5,6-dihydro-8H-imidazo[1,5-a]pyrazine-7-carboxylic acid tert-butyl ester (1.150 g; 2.28 mmol) and subsequent purification by FC (DCM/MeOH=50/1) afforded 1-ethyl-3-trifluoromethyl-8-[2-(4-trifluoromethyl-phenyl)-ethyl]-5,6-dihydro-8H-imidazo[1,5-a]pyrazine-7-carboxylic acid tert-butyl ester as a brown solid (0.455 g; 40%). LC-MS: t_{R}=1.17 min.; [M+H]⁺: 492.36 g/mol.

1-ethyl-8-[2-(3-fluoro-4-trifluoromethyl-phenyl)-ethyl]-3-trifluoromethyl-5,6-dihydro-8H-imidazo[1,5-a]pyrazine-7-carboxylic acid tert-butyl ester

According to the described general procedure (GP18A), trifluoromethylation (80° C.; 5.5 h) of 3-bromo-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester (1.083 g; 2.08 mmol) and subsequent purification by FC (DCM/MeOH=50/1) afforded 1-ethyl-8-[2-(3-fluoro-4-trifluoromethyl-phenyl)-ethyl]-3-trifluoromethyl-5,6-dihydro-8H-imidazo[1,5-a]pyrazine-7-carboxylic acid tert-butyl ester as a yellow oil (0.472 g; 45%). LC-MS: t_{R}=1.18 min.; [M+H]⁺: 510.26 g/mol.

### D.4.2 Second General Procedure for Trifluoromethylation of Imidazoles

3-cyclopropyl-8-[2-(3-fluoro-4-trifluoromethyl-phenyl)-ethyl]-1-trifluoromethyl-5,6-dihydro-8H-imidazo[1,5-a]pyrazine-7-carboxylic acid tert-butyl ester [General Procedure for trifluoromethylation of 5,6,7,8-tetrahydro-imidazo[1,5-a]pyrazine Derivatives (GP18B)]

A solution of 3-cyclopropyl-8-[2-(3-fluoro-4-trifluoromethyl-phenyl)-ethyl]-1-iodo-5,6-dihydro-8H-imidazo[1,5-a]pyrazine-7-carboxylic acid tert-butyl ester (938 mg; 1.62 mmol; 1.0 eq.) in anh. DMF (45 ml) was treated successively at rt with copper(I) iodide CuI (1.543 g; 8.10 mmol; 5.0 eq.), hexamethylphosphoramide (2.82 ml; 16.20 mmol; 10.0 eq.), and finally with methyl 2,2-difluoro-2-(fluorosulfonyl)acetate (1.34 ml; 10.53 mmol; 6.5 eq.). The resulting heterogeneous mixture was heated to 80° C., under nitrogen, for 8 h. After cooling to rt, water (150 ml), and Et₂O (250 ml) were carefully added. The yellow organic layer was further washed with water (3×75 ml), dried over anh. MgSO₄, filtered, and concentrated to dryness under reduced pressure. The resulting crude was purified by FC (DCM/MeOH=100/1) to give 3-cyclopropyl-8-[2-(3-fluoro-4-trifluoromethyl-phenyl)-ethyl]-1-trifluoromethyl-5,6-dihydro-8H-imidazo[1,5-a]pyrazine-7-carboxylic acid tert-butyl ester as a colorless solid which was further dried under HV (523 mg; 62%). LC-MS: t_{R}=1.16 min.; [M+H]⁺: 522.44 g/mol.

3-methylsulfanyl-1-trifluoromethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP18B), trifluoromethylation (80° C.; 5 h 45) of 1-iodo-3-methylsulfanyl-8-[2-(4-trifluoromethyl-phenyl)-ethyl]-5,6-dihydro-8H-imidazo[1,5-a]pyrazine-7-carboxylic acid tert-butyl ester (7.540 g; 13.28 mmol), and subsequent purification by FC (DCM/MeOH=100/1) afforded 3-methylsulfanyl-1-trifluoromethyl-8-[2-(4-trifluoromethyl-phenyl)-ethyl]-5,6-dihydro-8H-imidazo[1,5-a]pyrazine-7-carboxylic acid tert-butyl ester as a pale yellow solid (3.650 g; 54%). LC-MS: t_{R}=1.18 min.; [M+H]⁺: 510.39 g/mol.

D.5 Introduction of alkylthio Substituents —S—(C₁₋₄)alkyl
3-ethyl-1-methylsulfanyl-8-[2-(4-trifluoromethyl-phenyl)-ethyl]-5,6-dihydro-8H-imidazo[1,5-a]pyrazine-7-carboxylic acid tert-butyl ester [General Procedure for Introduction of alkylthio substituents —S—(C₁₋₄)alkyl (GP 19)]

A cooled (0° C.) mixture of 3-ethyl-1-iodo-8-[2-(4-trifluoromethyl-phenyl)-ethyl]-5,6-dihydro-8H-imidazo[1,5-a]pyrazine-7-carboxylic acid tert-butyl ester (3.390 g; 6.17 mmol; 1.0 eq.) in NMP (34 ml) was treated with sodium thiomethoxide (1.643 g; 23.44 mmol; 3.8 eq.), and copper(I) chloride CuCl (733 mg; 7.40 mmol; 1.2 eq.). The resulting mixture was then heated to 140° C. for 1 h. After cooling to rt, 25% NH₄OH in water (21 ml) was added, and the product was extracted with DCM (3×75 ml). The organic layer was dried over anh. MgSO₄, filtered, and concentrated to dryness under reduced pressure. The obtained crude was finally purified by FC (DCM/MeOH=20/1) to give 3-ethyl-1-methylsulfanyl-8-[2-(4-trifluoromethyl-phenyl)-ethyl]-5,6-dihydro-8H-imidazo[1,5-a]pyrazine-7-carboxylic acid tert-butyl ester as a dark-yellow oil which was further dried under HV (2.000 g; 69%). LC-MS: t_{R}=0.97 min.; [M+H]⁺: 470.38 g/mol.

D.6 Oxidation of alkylthio-substituents —S—(C₁₋₄)alkyl to the Corresponding alkylsulfonyl-substituents —SO₂—(C₁₋₄)alkyl
3-methanesulfonyl-1-trifluoromethyl-8-[2-(4-trifluoromethyl-phenyl)-ethyl]-5,6-dihydro-8H-imidazo[1,5-a]pyrazine-7-carboxylic acid tert-butyl ester
[General Procedure for Oxidation of alkylthio-substituents —S—(C₁₋₄)alkyl to the Corresponding alkylsulfonyl-substituents —SO₂—(C₁₋₄)alkyl (GP20)]

A solution of 3-methylsulfanyl-1-trifluoromethyl-8-[2-(4-trifluoromethyl-phenyl)-ethyl]-5,6-dihydro-8H-imidazo[1,5-a]pyrazine-7-carboxylic acid tert-butyl ester (3.650 g; 7.16 mmol; 1.0 eq.) in DCM (100 ml) was treated with MCPBA (3.708 g; 21.49 mmol; 3.0 eq.), and the resulting solution was stirred at rt, under nitrogen, for 15 h. The reaction mixture was diluted with DCM (200 ml), and was washed with aq. saturated NaHCO₃ (200 ml). The organic layer was dried over anh. MgSO₄, filtered, and concentrated to dryness under reduced pressure. The resulting crude was purified by FC (DCM/MeOH=50/1) to give 3-methanesulfonyl-1-trifluoromethyl-8-[2-(4-trifluoromethyl-phenyl)-ethyl]-5,6-dihydro-8H-imidazo[1,5-a]pyrazine-7-carboxylic acid tert-butyl ester as a colorless solid which was further dried under HV (3.410 g; 88%). LC-MS: t_{R}=1.16 min.; [M+H]⁺: 542.13 g/mol.

1-iodo-3-methanesulfonyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP20), MCPBA-mediated oxidation of 1-iodo-3-methylsulfanyl-8-[2-(4-trifluoromethyl-phenyl)-ethyl]-5,6-dihydro-8H-imidazo[1,5-a]pyrazine-7-carboxylic acid tert-butyl ester (300 mg; 0.52 mmol), and subsequent purification by FC (DCM/MeOH=100/1) afforded 1-iodo-3-methanesulfonyl-8-[2-(4-trifluoromethyl-phenyl)-ethyl]-5,6-dihydro-8H-imidazo[1,5-a]pyrazine-7-carboxylic acid tert-butyl ester as a pale yellow solid (185 mg; 58%). LC-MS: t_{R}=1.13 min.; [M+H]⁺: 600.08 g/mol.

3-ethyl-1-methanesulfonyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP20), MCPBA-mediated oxidation (rt; 6 h) of 3-ethyl-1-methylsulfanyl-8-[2-(4-trifluoromethyl-phenyl)-ethyl]-5,6-dihydro-8H-imidazo[1,5-a]pyrazine-7-carboxylic acid tert-butyl ester (1.350 g; 2.87 mmol), and subsequent purification by FC (DCM/MeOH=20/1) afforded 3-ethyl-1-methanesulfonyl-8-[2-(4-trifluoromethyl-phenyl)-ethyl]-5,6-dihydro-8H-imidazo[1,5-a]pyrazine-7-carboxylic acid tert-butyl ester as a colorless solid (740 mg; 51%). LC-MS: t_{R}=1.07 min.; [M+H]⁺: 502.54 g/mol.

D.7 Introduction of alkyl Substituents Via Stine Cross-Coupling Reaction, and Subsequent Hydrogenation
3-cyclopropyl-8-[2-(3-fluoro-4-trifluoromethyl-phenyl)-ethyl]-1-vinyl-5,6-dihydro-8H-imidazo[1,5-a]pyrazine-7-carboxylic acid tert-butyl ester [General Procedure for Stine Cross-Coupling Reaction (GP21)]

A solution of 3-cyclopropyl-8-[2-(3-fluoro-4-trifluoromethyl-phenyl)-ethyl]-1-iodo-5,6-dihydro-8H-imidazo[1,5-a]pyrazine-7-carboxylic acid tert-butyl ester (950 mg; 1.64 mmol; 1.0 eq.) in anh. DMF (9 ml) was treated successively at rt with tris(dibenzylideneacetone)dipalladium(0) Pd₂ dba₃ (48 mg; 0.05 mmol; 0.032 eq.), triphenylphosphine (54 mg; 0.20 mmol; 0.125 eq.), and finally with tributyl(vinyl)tin (1.0 ml; 2.0 eq.). The resulting mixture was heated to 90° C., under nitrogen, for 35 h. After cooling to rt, AcOEt (125 ml), water (75 ml), and brine (25 ml) were added. The resulting orange organic layer was dried over anh. MgSO₄, filtered, and concentrated to dryness under reduced pressure. The crude was purified by FC (DCM/MeOH=40/1) to afford 3-cyclopropyl-8-[2-(3-fluoro-4-trifluoromethyl-phenyl)-ethyl]-1-vinyl-5,6-dihydro-8H-imidazo[1,5-a]pyrazine-7-carboxylic acid tert-butyl ester as a yellow oil which was further dried under HV (494 mg; 63%). LC-MS: t_{R}=0.98 min.; [M+H]⁺: 480.06 g/mol.

3-methylsulfanyl-8-[2-(4-trifluoromethyl-phenyl)-ethyl]-1-vinyl-5,6-dihydro-8H-imidazo[1,5-a]pyrazine-7-carboxylic acid tert-butyl ester

According to the described general procedure (GP21), Stille cross-coupling reaction (90° C.; 6 h) with 1-iodo-3-methylsulfanyl-8-[2-(4-trifluoromethyl-phenyl)-ethyl]-5,6-dihydro-8H-imidazo[1,5-a]pyrazine-7-carboxylic acid tert-butyl ester (1.300 g; 2.29 mmol), and subsequent purification by FC (AcOEt/heptane=2/3) afforded 3-methylsulfanyl-8-[2-(4-trifluoromethyl-phenyl)-ethyl]-1-vinyl-5,6-dihydro-8H-imidazo[1,5-a]pyrazine-7-carboxylic acid tert-butyl ester as a pale yellow oil (860 mg; 80%). LC-MS: t_{R}=0.99 min.; [M+H]⁺: 468.38 g/mol.

8-[2-(4-trifluoromethyl-phenyl)-ethyl]-1-vinyl-5,6-dihydro-8H-imidazo[1,5-a]pyrazine-7-carboxylic acid tert-butyl ester

According to the described general procedure (GP21), Stille cross-coupling reaction (90° C.; 20 h) with 1-iodo-8-[2-(4-trifluoromethyl-phenyl)-ethyl]-5,6-dihydro-8H-imidazo[1,5-a]pyrazine-7-carboxylic acid tert-butyl ester (8.660 g; 16.61 mmol), and subsequent purification by FC (DCM/MeOH=50/1) afforded 8-[2-(4-trifluoromethyl-phenyl)-ethyl]-1-vinyl-5,6-dihydro-8H-imidazo[1,5-a]pyrazine-7-carboxylic acid tert-butyl ester as a pale yellow solid (2.608 g; 37%). LC-MS: t_{R}=0.94 min.; [M+H]⁺: 422.38 g/mol.

[2-(4-vinyl-imidazol-1-yl)-ethyl]carbamic acid tert-butyl ester

According to the described general procedure (GP21), Stille cross-coupling reaction (90° C.; 17 h) with [2-(4-iodo-imidazol-1-yl)-ethyl]-carbamic acid tert-butyl ester (3.640 g; 10.79 mmol), and subsequent purification by FC (DCM/MeOH=19/1) afforded [2-(4-vinyl-imidazol-1-yl)-ethyl]-carbamic acid tert-butyl ester as a pale yellow solid (1.960 g; 77%). LC-MS: t_{R}=0.66 min.; [M+H]⁺: 238.50 g/mol.

3-cyclopropyl-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester [General Procedure for Hydrogenation of Olefins Connected to Imidazoles (GP22)]

A mixture of 3-cyclopropyl-8-[2-(3-fluoro-4-trifluoromethyl-phenyl)-ethyl]-1-vinyl-5,6-dihydro-8H-imidazo[1,5-a]pyrazine-7-carboxylic acid tert-butyl ester (258 mg; 0.53 mmol), and 10% palladium on activated charcoal (250 mg) was placed under nitrogen before addition of MeOH (5 ml). This suspension was placed under vacuum, then under hydrogen (1 atm), and stirring at rt was continued for 11 h. Filtration over a pad of celite, concentration to dryness under reduced pressure, and additional drying under HV afforded 3-cyclopropyl-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester as a colorless solid (223 mg; 86%). LC-MS: t_{R}=0.96 min.; [M+H]⁺: 482.14 g/mol.

1-ethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP22), hydrogenation (rt; 15 h) of 8-[2-(4-trifluoromethyl-phenyl)-ethyl]-1-vinyl-5,6-dihydro-8H-imidazo[1,5-a]pyrazine-7-carboxylic acid tert-butyl ester (2.608 g; 6.18 mmol) afforded 1-ethyl-8-[2-(4-trifluoromethyl-phenyl)-ethyl]-5,6-dihydro-8H-imidazo[1,5-a]pyrazine-7-carboxylic acid tert-butyl ester as a pale yellow solid (2.530 g; 97%). LC-MS: t_{R}=0.95 min.; [M+H]⁺: 424.36 g/mol.

[2-(4-ethyl-imidazol-1-yl)-ethyl]-carbamic acid tert-butyl ester

According to the described general procedure (GP22), hydrogenation (rt; 4 h) of [2-(4-vinyl-imidazol-1-yl)-ethyl]-carbamic acid tert-butyl ester (2.100 g; 8.84 mmol) afforded [2-(4-ethyl-imidazol-1-yl)-ethyl]-carbamic acid tert-butyl ester as a yellow oil (1.970 g; 93%). LC-MS: t_{R}=0.65 min.; [M+H]⁺: 240.47 g/mol.

D.8 Insertion of Alkoxy Substituents (C₁₋₄)alkoxy
3-ethoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester [General Procedure for Insertion of Alkoxy Substituents (C₁₋₄)alkoxy (GP23)]

A solution of 3-methanesulfonyl-1-trifluoromethyl-8-[2-(4-trifluoromethyl-phenyl)-ethyl]-5,6-dihydro-8H-imidazo[1,5-a]pyrazine-7-carboxylic acid tert-butyl ester (50 mg; 0.09 mmol; 1.0 eq.) in anh. EtOH (2.0 ml) was treated with sodium ethoxide (83.8 mg; 0.46 mmol; 5.0 eq.), and the resulting mixture was heated to 80° C., under nitrogen, for 3 h. After cooling to rt, the reaction mixture was concentrated to dryness under reduced pressure. Dichloromethane (50 ml), and water (50 ml) were added, and the organic layer was then dried over anh. MgSO₄, filtered, and concentrated to dryness under reduced pressure. Purification by FC (DCM/MeOH=50/1) afforded 3-ethoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester as a pale yellow solid which was further dried under HV (39.5 mg; 84%). LC-MS: t_{R}=1.19 min.; [M+H]⁺: 508.49 g/mol.

3-methoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester

According to the described general procedure (GP23), methoxylation (60° C.; 6 h) of 3-methanesulfonyl-1-trifluoromethyl-8-[2-(4-trifluoromethyl-phenyl)-ethyl]-5,6-dihydro-8H-imidazo[1,5-a]pyrazine-7-carboxylic acid tert-butyl ester (100 mg; 0.18 mmol) in anh. MeOH (5 ml), and subsequent purification by FC (DCM/MeOH=50/1) afforded 3-methoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester as a colorless solid (90 mg; 99%). LC-MS: t_{R}=1.16 min.; [M+H]⁺: 494.23 g/mol.

D.9 Boc-deprotection of 5,6,7,8-tetrahydro-imidazo[1,5-a]pyrazine Derivatives
1-chloro-3-cyclopropyl-8-[2-(4-difluoromethoxy-phenyl)-ethyl]-5,6,7,8-tetrahydro-imidazo[1,5-a]pyrazine [General Procedure for Boc-deprotection of 5,6,7,8-tetrahydro-imidazo[1,5-a]pyrazine Derivatives (GP24)]

An ice-cooled solution of 1-chloro-3-cyclopropyl-8-[2-(4-difluoromethoxy-phenyl)-ethyl]-5,6-dihydro-8H-imidazo[1,5-a]pyrazine-7-carboxylic acid tert-butyl ester (447 mg; 0.95 mmol; 1.0 eq.) in anh. DCM (10 ml) was treated dropwise with a solution of 4N HCl in 1,4-dioxane (4.8 ml; 19.10 mmol; 20 eq.). The resulting suspension was further stirred at 0° C. for 10 min., and then at rt for 5 h 15. The heterogeneous reaction mixture was concentrated to dryness under reduced pressure, and the resulting yellow solid residue was dissolved in DCM (100 ml), and water (35 ml). Na₂CO₃ (475 mg; 4.48 mmol; 4.7 eq.) was then added portionwise, and the aq. layer was further extracted with DCM (50 ml). The mixed organic layers were then dried over anh. MgSO₄, filtered, and concentrated to dryness under reduced pressure. The resulting crude was purified by FC (DCM/MeOH/25% aq. NH₄OH=250/10/1) in order to isolate 1-chloro-3-cyclopropyl-8-[2-(4-difluoromethoxy-phenyl)-ethyl]-5,6,7,8-tetrahydro-imidazo[1,5-a]pyrazine as a yellow oil which was further dried under HV (338 mg; 96%). LC-MS: t_{R}=0.80 min.; [M+H]⁺: 368.34 g/mol.

1-chloro-3-cyclopropyl-8-[2-(3-fluoro-4-trifluoromethyl-phenyl)-ethyl]-5,6,7,8-tetrahydro-imidazo[1,5-a]pyrazine

According to the general procedure (GP24), Boc-deprotection (rt; 2 h) of 1-chloro-3-cyclopropyl-8-[2-(3-fluoro-4-trifluoromethyl-phenyl)-ethyl]-5,6-dihydro-8H-imidazo[1,5-a]pyrazine-7-carboxylic acid tert-butyl ester (503 mg; 1.03 mmol) afforded after purification by FC (DCM/MeOH/25% aq. NH₄OH=150/10/1) the target molecule 1-chloro-3-cyclopropyl-8-[2-(3-fluoro-4-trifluoromethyl-phenyl)-ethyl]-5,6,7,8-tetrahydro-imidazo[1,5-a]pyrazine as a pale yellow solid (388 mg; 100%). LC-MS: t_{R}=0.89 min.; [M+H]⁺: 388.31 g/mol.

1-chloro-3-cyclopropyl-8-[2-(4-fluoro-3-trifluoromethyl-phenyl)-ethyl]-5,6,7,8-tetrahydro-imidazo[1,5-a]pyrazine

According to the general procedure (GP24), Boc-deprotection (rt; 2 h 30) of 1-chloro-3-cyclopropyl-8-[2-(4-fluoro-3-trifluoromethyl-phenyl)-ethyl]-5,6-dihydro-8H-imidazo[1,5-a]pyrazine-7-carboxylic acid tert-butyl ester (674.3 mg; 1.38 mmol) afforded after purification by FC (DCM/MeOH/25% aq. NH₄OH=150/10/1) the target molecule 1-chloro-3-cyclopropyl-8-[2-(4-fluoro-3-trifluoromethyl-phenyl)-ethyl]-5,6,7,8-tetrahydro-imidazo[1,5-a]pyrazine as a pale yellow oil (483.6 mg; 90%). LC-MS: t_{R}=0.83 min.; [M+H]⁺: 388.29 g/mol.

1-chloro-8-[2-(4-chloro-3-fluoro-phenyl)-ethyl]-3-cyclopropyl-5,6,7,8-tetrahydro-imidazo[1,5-a]pyrazine

According to the general procedure (GP24), Boc-deprotection (rt; 24 h) of 1-chloro-8-[2-(4-chloro-3-fluoro-phenyl)-ethyl]-3-cyclopropyl-5,6-dihydro-8H-imidazo[1,5-a]pyrazine-7-carboxylic acid tert-butyl ester (670 mg; 1.47 mmol) afforded after purification by FC (DCM/MeOH/25% aq. NH₄OH=150/10/1) the target molecule 1-chloro-8-[2-(4-chloro-3-fluoro-phenyl)-ethyl]-3-cyclopropyl-5,6,7,8-tetrahydro-imidazo[1,5-a]pyrazine as a yellow oil (340 mg; 65%). LC-MS: t_{R}=0.80 min.; [M+H]⁺: 354.23 g/mol.

1-chloro-3-cyclopropyl-8-[2-(3-fluoro-4-trifluoromethoxy-phenyl)-ethyl]-5,6,7,8-tetrahydro-imidazo[1,5-a]pyrazine

According to the general procedure (GP24), Boc-deprotection (rt; 2 h) of 1-chloro-3-cyclopropyl-8-[2-(3-fluoro-4-trifluoromethoxy-phenyl)-ethyl]-5,6-dihydro-8H-imidazo[1,5-a]pyrazine-7-carboxylic acid tert-butyl ester (1.254 g; 2.48 mmol) afforded after purification by FC (DCM/MeOH/25% aq. NH₄OH=150/10/1) the target molecule 1-chloro-3-cyclopropyl-8-[2-(3-fluoro-4-trifluoromethoxy-phenyl)-ethyl]-5,6,7,8-tetrahydro-imidazo[1,5-a]pyrazine as a pale yellow oil (834 mg; 83%). LC-MS: t_{R}=0.85 min.; [M+H]⁺: 404.27 g/mol.

1-chloro-3-cyclopropyl-8-[2-(4-difluoromethoxy-3-fluoro-phenyl)-ethyl]-5,6,7,8-tetrahydro-imidazo[1,5-a]pyrazine

According to the general procedure (GP24), Boc-deprotection (rt; 3 h) of 1-chloro-3-cyclopropyl-8-[2-(4-difluoromethoxy-3-fluoro-phenyl)-ethyl]-5,6-dihydro-8H-imidazo[1,5-a]pyrazine-7-carboxylic acid tert-butyl ester (558 mg; 1.14 mmol) afforded after purification by FC (DCM/MeOH/25% aq. NH₄OH=150/10/1) the target molecule 1-chloro-3-cyclopropyl-8-[2-(4-difluoromethoxy-3-fluoro-phenyl)-ethyl]-5,6,7,8-tetrahydro-imidazo[1,5-a]pyrazine as a pale yellow oil (461 mg; 100%). LC-MS: t_{R}=0.81 min.; [M+H]⁺: 386.28 g/mol.

1-chloro-3-methylsulfanyl-8-[2-(4-trifluoromethyl-phenyl)-ethyl]-5,6,7,8-tetrahydro-imidazo[1,5-a]pyrazine

According to the general procedure (GP24), Boc-deprotection (rt; 6.5 h) of 1-chloro-3-methylsulfanyl-8-[2-(4-trifluoromethyl-phenyl)-ethyl]-5,6-dihydro-8H-imidazo[1,5-a]pyrazine-7-carboxylic acid tert-butyl ester (339 mg; 0.71 mmol) afforded after purification by FC (DCM/MeOH/25% aq. NH₄OH=250/10/1) the target molecule 1-chloro-3-methylsulfanyl-8-[2-(4-trifluoromethyl-phenyl)-ethyl]-5,6,7,8-tetrahydro-imidazo[1,5-a]pyrazine as a slightly yellow oil (252 mg; 94%). LC-MS: t_{R}=0.84 min.; [M+H]⁺: 376.34 g/mol.

1-chloro-8-[2-(2,3-difluoro-4-trifluoromethyl-phenyl)-ethyl]-3-methylsulfanyl-5,6,7,8-tetrahydro-imidazo[1,5-a]pyrazine

According to the described general procedure (GP24), Boc-deprotection (rt; 3 h) of 1-chloro-8-[2-(2,3-difluoro-4-trifluoromethyl-phenyl)-ethyl]-3-methylsulfanyl-5,6-dihydro-8H-imidazo[1,5-a]pyrazine-7-carboxylic acid tert-butyl ester (970 mg; 1.89 mmol) afforded after purification by FC (DCM/MeOH/25% aq. NH₄OH=150/10/1) the target molecule 1-chloro-8-[2-(2,3-difluoro-4-trifluoromethyl-phenyl)-ethyl]-3-methylsulfanyl-5,6,7,8-tetrahydro-imidazo[1,5-a]pyrazine as a yellow oil (729 mg; 93%). LC-MS: t_{R}=0.86 min.; [M+H]⁺: 411.95 g/mol.

1-chloro-8-[2-(4-difluoromethoxy-phenyl)-ethyl]-3-ethyl-5,6,7,8-tetrahydro-imidazo[1,5-a]pyrazine

According to the described general procedure (GP24), Boc-deprotection (rt; 2 h) of 1-chloro-8-[2-(4-difluoromethoxy-phenyl)-ethyl]-3-ethyl-5,6-dihydro-8H-imidazo[1,5-a]pyrazine-7-carboxylic acid tert-butyl ester (2.070 g; 4.54 mmol) afforded after purification by FC (DCM/MeOH/25% aq. NH₄OH=150/10/1.5) the target molecule 1-chloro-8-[2-(4-difluoromethoxy-phenyl)-ethyl]-3-ethyl-5,6,7,8-tetrahydro-imidazo[1,5-a]pyrazine as a yellow oil (1.530 g; 95%). LC-MS: t_{R}=0.78 min.; [M+H]⁺: 356.33 g/mol.

1-chloro-8-[2-(4-difluoromethoxy-3-fluoro-phenyl)-ethyl]-3-ethyl-5,6,7,8-tetrahydro-imidazo[1,5-a]pyrazine

According to the described general procedure (GP24), Boc-deprotection (rt; 2 h) of 1-chloro-8-[2-(4-difluoromethoxy-3-fluoro-phenyl)-ethyl]-3-ethyl-5,6-dihydro-8H-imidazo[1,5-a]pyrazine-7-carboxylic acid tert-butyl ester (3.100 g; 6.54 mmol) afforded after purification by FC (DCM/MeOH/25% aq. NH₄OH=150/10/1) the target molecule 1-chloro-8-[2-(4-difluoromethoxy-3-fluoro-phenyl)-ethyl]-3-ethyl-5,6,7,8-tetrahydro-imidazo[1,5-a]pyrazine as a yellow oil (2.260 g; 92%). LC-MS: t_{R}=0.80 min.; [M+H]⁺: 374.28 g/mol.

1-chloro-8-[2-(3-difluoromethoxy-phenyl)-ethyl]-3-ethyl-5,6,7,8-tetrahydro-imidazo[1,5-a]pyrazine

According to the described general procedure (GP24), Boc-deprotection (rt; 3 h) of 1-chloro-8-[2-(3-difluoromethoxy-phenyl)-ethyl]-3-ethyl-5,6-dihydro-8H-imidazo[1,5-a]pyrazine-7-carboxylic acid tert-butyl ester (580 mg; 1.27 mmol) afforded after purification by FC (DCM/MeOH/25% aq. NH₄OH=150/10/1) the target molecule 1-chloro-8-[2-(3-difluoromethoxy-phenyl)-ethyl]-3-ethyl-5,6,7,8-tetrahydro-imidazo[1,5-a]pyrazine as a yellow oil (380 mg; 84%). LC-MS: t_{R}=0.79 min.; [M+H]⁺: 356.34 g/mol.

1-chloro-8-[2-(4-difluoromethoxy-phenyl)-ethyl]-3-methyl-5,6,7,8-tetrahydro-imidazo[1,5-a]pyrazine

According to the described general procedure (GP24), Boc-deprotection (rt; 3 h) of 1-chloro-8-[2-(4-difluoromethoxy-phenyl)-ethyl]-3-methyl-5,6-dihydro-8H-imidazo[1,5-a]pyrazine-7-carboxylic acid tert-butyl ester (1.100 g; 2.48 mmol) afforded after purification by FC (DCM/MeOH/25% aq. NH₄OH=150/10/1) the target molecule 1-chloro-8-[2-(4-difluoromethoxy-phenyl)-ethyl]-3-methyl-5,6,7,8-tetrahydro-imidazo[1,5-a]pyrazine as a yellow oil (830 mg; 98%). LC-MS: t_{R}=0.77 min.; [M+H]⁺: 342.40 g/mol.

1-methyl-3-trifluoromethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 8 h) of 1-methyl-3-trifluoromethyl-8-[2-(4-trifluoromethyl-phenyl)-ethyl]-5,6-dihydro-8H-imidazo[1,5-a]pyrazine-7-carboxylic acid tert-butyl ester (203 mg; 0.42 mmol) afforded the target molecule 1-methyl-3-trifluoromethyl-8-[2-(4-trifluoromethyl-phenyl)-ethyl]-5,6,7,8-tetrahydro-imidazo[1,5-a]pyrazine as a slightly beige solid (95 mg; 59%). LC-MS: t_{R}=0.83 min.; [M+H]⁺: 378.26 g/mol.

3-cyclopropyl-8-[2-(3-fluoro-4-trifluoromethyl-phenyl)-ethyl]-1-trifluoromethyl-5,6,7,8-tetrahydro-imidazo[1,5-a]pyrazine

According to the described general procedure (GP24), Boc-deprotection (rt; 24 h) of 3-cyclopropyl-8-[2-(3-fluoro-4-trifluoromethyl-phenyl)-ethyl]-1-trifluoromethyl-5,6-dihydro-8H-imidazo[1,5-a]pyrazine-7-carboxylic acid tert-butyl ester (507 mg; 0.97 mmol) afforded the target molecule 3-cyclopropyl-8-[2-(3-fluoro-4-trifluoromethyl-phenyl)-ethyl]-1-trifluoromethyl-5,6,7,8-tetrahydro-imidazo[1,5-a]pyrazine as a beige solid (373 mg; 91%). LC-MS: t_{R}=0.87 min.; [M+H]⁺: 421.97 g/mol.

3-methylsulfanyl-1-trifluoromethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 2.5 h) of 3-methylsulfanyl-1-trifluoromethyl-8-[2-(4-trifluoromethyl-phenyl)-ethyl]-5,6-dihydro-8H-imidazo[1,5-a]pyrazine-7-carboxylic acid tert-butyl ester (181 mg; 0.35 mmol) afforded the target molecule 3-methylsulfanyl-1-trifluoromethyl-8-[2-(4-trifluoromethyl-phenyl)-ethyl]-5,6,7,8-tetrahydro-imidazo[1,5-a]pyrazine as a pale yellow oil (146 mg; 100%). LC-MS: t_{R}=0.88 min.; [M+H]⁺: 410.15 g/mol.

3-cyclopropyl-1-ethyl-8-[2-(3-fluoro-4-trifluoromethyl-phenyl)-ethyl]-5,6,7,8-tetrahydro-imidazo[1,5-a]pyrazine

According to the described general procedure (GP24), Boc-deprotection (rt; 14 h) of 3-cyclopropyl-1-ethyl-8-[2-(3-fluoro-4-trifluoromethyl-phenyl)-ethyl]-5,6-dihydro-8H-imidazo[1,5-a]pyrazine-7-carboxylic acid tert-butyl ester (217 mg; 0.45 mmol) afforded the target molecule 3-cyclopropyl-1-ethyl-8-[2-(3-fluoro-4-trifluoromethyl-phenyl)-ethyl]-5,6,7,8-tetrahydro-imidazo[1,5-a]pyrazine as a yellow oil (170 mg; 99%). LC-MS: t_{R}=0.74 min.; [M+H]⁺: 382.37 g/mol.

1-iodo-3-methanesulfonyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 2.5 h) of 1-iodo-3-methanesulfonyl-8-[2-(4-trifluoromethyl-phenyl)-ethyl]-5,6-dihydro-8H-imidazo[1,5-a]pyrazine-7-carboxylic acid tert-butyl ester (185 mg; 0.30 mmol) afforded the target molecule 1-iodo-3-methanesulfonyl-8-[2-(4-trifluoromethyl-phenyl)-ethyl]-5,6,7,8-tetrahydro-imidazo[1,5-a]pyrazine as a pale yellow oil (153 mg; 99%). LC-MS: t_{R}=0.83 min.; [M+H]⁺: 500.18 g/mol.

3-cyclopropyl-8-[2-(3-fluoro-4-trifluoromethyl-phenyl)-ethyl]-1-vinyl-5,6,7,8-tetrahydro-imidazo[1,5-a]pyrazine

According to the described general procedure (GP24), Boc-deprotection (rt; 15 h) of 3-cyclopropyl-8-[2-(3-fluoro-4-trifluoromethyl-phenyl)-ethyl]-1-vinyl-5,6-dihydro-8H-imidazo[1,5-a]pyrazine-7-carboxylic acid tert-butyl ester (135 mg; 0.28 mmol) afforded the target molecule 3-cyclopropyl-8-[2-(3-fluoro-4-trifluoromethyl-phenyl)-ethyl]-1-vinyl-5,6,7,8-tetrahydro-imidazo[1,5-a]pyrazine as an off-white solid (98 mg; 92%). LC-MS: t_{R}=0.75 min.; [M+H]⁺: 380.43 g/mol.

3-methylsulfanyl-8-[2-(4-trifluoromethyl-phenyl)-ethyl]-1-vinyl-5,6,7,8-tetrahydro-imidazo[1,5-a]pyrazine

According to the described general procedure (GP24), Boc-deprotection (rt; 2.5 h) of 3-methylsulfanyl-8-[2-(4-trifluoromethyl-phenyl)-ethyl]-1-vinyl-5,6-dihydro-8H-imidazo[1,5-a]pyrazine-7-carboxylic acid tert-butyl ester (800 mg; 1.71 mmol) afforded the target molecule 3-methylsulfanyl-8-[2-(4-trifluoromethyl-phenyl)-ethyl]-1-vinyl-5,6,7,8-tetrahydro-imidazo[1,5-a]pyrazine as a yellow solid (594 mg; 94%). LC-MS: t_{R}=0.79 min.; [M+H]⁺: 368.34 g/mol.

3-ethyl-1-methylsulfanyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 6 h) of 3-ethyl-1-methylsulfanyl-8-[2-(4-trifluoromethyl-phenyl)-ethyl]-5,6-dihydro-8H-imidazo[1,5-a]pyrazine-7-carboxylic acid tert-butyl ester (615 mg; 1.31 mmol) afforded the target molecule 3-ethyl-1-methylsulfanyl-8-[2-(4-trifluoromethyl-phenyl)-ethyl]-5,6,7,8-tetrahydro-imidazo[1,5-a]pyrazine as a yellow oil (340 mg; 70%). LC-MS: t_{R}=0.73 min.; [M+H]⁺: 370.48 g/mol.

3-ethoxy-1-trifluoromethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 3.5 h) of 3-ethoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester (78.9 mg; 0.15 mmol) afforded the target molecule 3-ethoxy-1-trifluoromethyl-8-[2-(4-trifluoromethyl-phenyl)-ethyl]-5,6,7,8-tetrahydro-imidazo[1,5-a]pyrazine as a pale yellow oil (44.6 mg; 70%). LC-MS: t_{R}=0.89 min.; [M+H]⁺: 408.14 g/mol.

1-chloro-3-cyclopropylmethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 15 h) of 1-chloro-3-cyclopropylmethyl-8-[2-(4-trifluoromethyl-phenyl)-ethyl]-5,6-dihydro-8H-imidazo[1,5-a]pyrazine-7-carboxylic acid tert-butyl ester (400 mg; 0.82 mmol) afforded the target molecule 1-chloro-3-cyclopropylmethyl-8-[2-(4-trifluoromethyl-phenyl)-ethyl]-5,6,7,8-tetrahydro-imidazo[1,5-a]pyrazine as an off-white solid (230 mg; 72%). LC-MS: t_{R}=0.80 min.; [M+H]⁺: 384.01 g/mol.

3-ethyl-1-methanesulfonyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 2 h) of 3-ethyl-1-methanesulfonyl-8-[2-(4-trifluoromethyl-phenyl)-ethyl]-5,6-dihydro-8H-imidazo[1,5-a]pyrazine-7-carboxylic acid tert-butyl ester (740 mg; 1.47 mmol) afforded the target molecule 3-ethyl-1-methanesulfonyl-8-[2-(4-trifluoromethyl-phenyl)-ethyl]-5,6,7,8-tetrahydro-imidazo[1,5-a]pyrazine as a yellow oil (520 mg; 88%). LC-MS: t_{R}=0.79 min.; [M+H]⁺: 402.05 g/mol.

3-methoxy-1-trifluoromethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 3.5 h) of 3-methoxy-1-trifluoromethyl-8-[2-(4-trifluoromethyl-phenyl)-ethyl]-5,6-dihydro-8H-imidazo[1,5-a]pyrazine-7-carboxylic acid tert-butyl ester (76.7 mg; 0.15 mmol) afforded the target molecule 3-methoxy-1-trifluoromethyl-8-[2-(4-trifluoromethyl-phenyl)-ethyl]-5,6,7,8-tetrahydro-imidazo[1,5-a]pyrazine as a yellow oil (55.7 mg; 91%). LC-MS: t_{R}=0.90 min.

1-chloro-3-cyclopropyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 4.5 h) of 1-chloro-3-cyclopropyl-8-[2-(4-trifluoromethyl-phenyl)-ethyl]-5,6-dihydro-8H-imidazo[1,5-a]pyrazine-7-carboxylic acid tert-butyl ester (7.730 g; 16.44 mmol) afforded the target molecule 1-chloro-3-cyclopropyl-8-[2-(4-trifluoromethyl-phenyl)-ethyl]-5,6,7,8-tetrahydro-imidazo[1,5-a]pyrazine as a yellow oil (6.080 g; 99%). LC-MS: t_{R}=0.83 min.; [M+H]⁺: 370.31 g/mol.

1-chloro-3-cyclopropyl-8-[2-(3-trifluoromethyl-phenyl)-ethyl]-5,6,7,8-tetrahydro-imidazo[1,5-a]pyrazine

According to the described general procedure (GP24), Boc-deprotection (rt; 3 h) of 1-chloro-3-cyclopropyl-8-[2-(3-trifluoromethyl-phenyl)-ethyl]-5,6-dihydro-8H-imidazo[1,5-a]pyrazine-7-carboxylic acid tert-butyl ester (2.150 g; 4.57 mmol) afforded the target molecule 1-chloro-3-cyclopropyl-8-[2-(3-trifluoromethyl-phenyl)-ethyl]-5,6,7,8-tetrahydro-imidazo[1,5-a]pyrazine as a yellow oil (1.360 g; 80%). LC-MS: t_{R}=0.83 min.; [M+H]⁺: 370.05 g/mol.

1-chloro-3-cyclopropyl-8-[2-(2-fluoro-4-trifluoromethyl-phenyl)-ethyl]-5,6,7,8-tetrahydro-imidazo[1,5-a]pyrazine

According to the described general procedure (GP24), Boc-deprotection (rt; 4 h 30) of 1-chloro-3-cyclopropyl-8-[2-(2-fluoro-4-trifluoromethyl-phenyl)-ethyl]-5,6-dihydro-8H-imidazo[1,5-a]pyrazine-7-carboxylic acid tert-butyl ester (2.810 g; 5.75 mmol) afforded the target molecule 1-chloro-3-cyclopropyl-8-[2-(2-fluoro-4-trifluoromethyl-phenyl)-ethyl]-5,6,7,8-tetrahydro-imidazo[1,5-a]pyrazine as a yellow oil (1.710 g; 77%). LC-MS: t_{R}=0.83 min.; [M+H]⁺: 388.16 g/mol.

1-ethyl-3-trifluoromethyl-8-[2-(4-trifluoromethyl-phenyl)-ethyl]-5,6,7,8-tetrahydro-imidazo[1,5-a]pyrazine

According to the described general procedure (GP24), Boc-deprotection (rt; 3 h) of 1-ethyl-3-trifluoromethyl-8-[2-(4-trifluoromethyl-phenyl)-ethyl]-5,6-dihydro-8H-imidazo[1,5-a]pyrazine-7-carboxylic acid tert-butyl ester (929 mg; 1.89 mmol) afforded the target molecule 1-ethyl-3-trifluoromethyl-8-[2-(4-trifluoromethyl-phenyl)-ethyl]-5,6,7,8-tetrahydro-imidazo[1,5-a]pyrazine as a yellow oil (740 mg; 100%). LC-MS: t_{R}=0.87 min.; [M+H]⁺: 391.99 g/mol.

1-ethyl-8-[2-(3-fluoro-4-trifluoromethyl-phenyl)-ethyl]-3-trifluoromethyl-5,6,7,8-tetrahydro-imidazo[1,5-a]pyrazine

According to the described general procedure (GP24), Boc-deprotection (rt; 3 h) of 1-ethyl-8-[2-(3-fluoro-4-trifluoromethyl-phenyl)-ethyl]-3-trifluoromethyl-5,6-dihydro-8H-imidazo[1,5-a]pyrazine-7-carboxylic acid tert-butyl ester (472 mg; 0.92 mmol) afforded the target molecule 1-ethyl-8-[2-(3-fluoro-4-trifluoromethyl-phenyl)-ethyl]-3-trifluoromethyl-5,6,7,8-tetrahydro-imidazo[1,5-a]pyrazine as a yellow oil (200 mg; 53%). LC-MS: t_{R}=0.88 min.; [M+H]⁺: 410.26 g/mol.

### E. Synthesis of Electrophiles Z—CHPh-C(O)NHR4

E.1 Synthesis of toluene-4-sulfonic acid (S)-methylcarbamoyl-phenyl-methyl ester
(S)-2-hydroxy-N-methyl-2-phenyl-acetamide

Methyl (S)-(+)-mandelate (17.000 g; 102.304 mmol) was dissolved in a 2.0 M solution of methylamine in MeOH (230 ml; 460 mmol) and kept at rt for 1 day. Another portion of methylamine in MeOH (10 ml; 20 mmol) was added. A third portion of methylamine in MeOH (10 ml; 20 mmol) was added one day later. After additional 24 h the reaction mixture was concentrated to dryness under reduced pressure to give the desired amide (S)-2-hydroxy-N-methyl-2-phenyl-acetamide as pale yellow crystals which were used without further purification. LC-MS: t_{R}=0.52 min.; [M+H]⁺=166 g/mol.

toluene-4-sulfonic acid (S)-methylcarbamoyl-phenyl-methyl ester

DIPEA (2.74 ml; 16.005 mmol) and DMAP (145 mg; 1.186 mmol) were successively added at rt to a solution of (S)-2-hydroxy-N-methyl-2-phenyl-acetamide (2.400 g; 14.528 mmol) in DCM (50 ml). The mixture was treated portionwise with TsCl (2.770 g; 14.529 mmol) and stirred at rt for 2 h. The solvent was removed in vacuo and the residue was dissolved in EA. The organic solution was then washed twice with an aq. sat. NaHCO₃ solution and once with brine. The solvents were removed in vacuo and the residue was recrystallized from EA/tert.-butylmethylether to give the expected tosylate derivative toluene-4-sulfonic acid (S)-methylcarbamoyl-phenyl-methyl ester as colorless crystals. LC-MS: t_{R}=0.93 min.; [M+H]⁺=320 g/mol.

### F. Synthesis of Example Compounds

N-alkylation of 5,6,7,8-tetrahydro-imidazo[1,5-a]pyrazine derivatives with tosylates
[General Procedure for N-Alkylation with Electrophiles (GP25)]

To a solution of the respective 5,6,7,8-tetrahydro-imidazo[1,5-a]pyrazine derivative (1.0 mmol of secondary amine) in 3-methyl-2-butanone (10 ml) were added successively N-ethyldiisopropylamine (2.0 mmol), and the respective tosylate (1.1 mmol). This mixture was then heated to 70° C., under nitrogen, for the indicated reaction time. After cooling to rt, Et₂O (125 ml), and water (35 ml) were added, and the organic layer was further washed with water (30 ml). The mixed aq. layers were extracted with Et₂O (2×30 ml). The combined organic layers were then dried over anh. MgSO₄, filtered, and concentrated to dryness under reduced pressure. The resulting crude was finally purified according to the indicated method.

### Example 1

### (R)-2′-{1-chloro-3-cyclopropyl-(S)-8-[2-(3-fluoro-4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-chloro-3-cyclopropyl-8-[2-(3-fluoro-4-trifluoromethyl-phenyl)-ethyl]-5,6,7,8-tetrahydro-imidazo[1,5-a]pyrazine (109.6 mg; 0.28 mmol) with toluene-4-sulfonic acid (S)-methylcarbamoyl-phenyl-methyl ester. Subsequent separation of diastereoisomers by preparative HPLC afforded the target compound as colorless solid. LC-MS: t_{R}=0.93 min.; [M+H]⁺: 535.39 g/mol.

### Example 2

### (R)-2′-{1-chloro-3-cyclopropyl-(S)-8-[2-(4-fluoro-3-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 97 h) of 1-chloro-3-cyclopropyl-8-[2-(4-fluoro-3-trifluoromethyl-phenyl)-ethyl]-5,6,7,8-tetrahydro-imidazo[1,5-a]pyrazine (483.6 mg; 1.24 mmol) with toluene-4-sulfonic acid (S)-methylcarbamoyl-phenyl-methyl ester. Subsequent separation of diastereoisomers by FC (DCM/MeOH=100/1) afforded the target compound as pale yellow solid. LC-MS: t_{R}=0.99 min.; [M+H]⁺: 535.36 g/mol.

### Example 3

### (R)-2′-{1-chloro-(S)-8-[2-(4-chloro-3-fluoro-phenyl)-ethyl]-3-cyclopropyl-5,6-dihydro-8H-imidazo[1,5-a]pyrazin-7-yl}-N-methyl-2′-phenyl-acetamide

Prepared according to the described general procedure (GP25) by reaction (70° C.; 72 h) of 1-chloro-8-[2-(4-chloro-3-fluoro-phenyl)-ethyl]-3-cyclopropyl-5,6,7,8-tetrahydro-imidazo[1,5-a]pyrazine (340 mg; 0.96 mmol) with toluene-4-sulfonic acid (S)-methylcarbamoyl-phenyl-methyl ester. Subsequent separation of diastereoisomers by FC (DCM/MeOH=50/1) afforded the target compound as colorless solid. LC-MS: t_{R}=0.98 min.; [M+H]⁺: 501.38 g/mol.

### Example 4

### (R)-2′-{1-chloro-3-cyclopropyl-(S)-8-[2-(4-difluoromethoxy-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 67 h) of 1-chloro-3-cyclopropyl-8-[2-(4-difluoromethoxy-phenyl)-ethyl]-5,6,7,8-tetrahydro-imidazo[1,5-a]pyrazine (329 mg; 0.89 mmol) with toluene-4-sulfonic acid (S)-methylcarbamoyl-phenyl-methyl ester. Subsequent separation of diastereoisomers by FC (DCM/MeOH=50/1) afforded the target compound as slightly beige solid. LC-MS: t_{R}=0.95 min.; [M+H]⁺: 515.41 g/mol.

### Example 5

### (R)-2′-{1-chloro-3-cyclopropyl-(S)-8-[2-(3-fluoro-4-trifluoromethoxy-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 96 h) of 1-chloro-3-cyclopropyl-8-[2-(3-fluoro-4-trifluoromethoxy-phenyl)-ethyl]-5,6,7,8-tetrahydro-imidazo[1,5-a]pyrazine (834 mg; 2.06 mmol) with toluene-4-sulfonic acid (S)-methylcarbamoyl-phenyl-methyl ester. Subsequent separation of diastereoisomers by FC (AcOEt/heptane=1/5 to AcOEt) afforded the target compound as pale yellow solid. LC-MS: t_{R}=1.01 min.; [M+H]⁺: 551.40 g/mol.

### Example 6

### (R)-2′-{1-chloro-3-cyclopropyl-(S)-8-[2-(4-difluoromethoxy-3-fluoro-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 96 h) of 1-chloro-3-cyclopropyl-8-[2-(4-difluoromethoxy-3-fluoro-phenyl)-ethyl]-5,6,7,8-tetrahydro-imidazo[1,5-a]pyrazine (461 mg; 1.19 mmol) with toluene-4-sulfonic acid (S)-methylcarbamoyl-phenyl-methyl ester. Subsequent separation of diastereoisomers by FC (AcOEt/heptane=1/5 to AcOEt) afforded the target compound as pale yellow solid. LC-MS: t_{R}=0.96 min.; [M+H]⁺: 533.40 g/mol.

### Example 7

### (R)-2′-{1-chloro-3-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 90.5 h) of 1-chloro-3-methylsulfanyl-8-[2-(4-trifluoromethyl-phenyl)-ethyl]-5,6,7,8-tetrahydro-imidazo[1,5-a]pyrazine (243 mg; 0.64 mmol) with toluene-4-sulfonic acid (S)-methylcarbamoyl-phenyl-methyl ester. Subsequent separation of diastereoisomers by FC (DCM/MeOH=50/1) afforded the target compound as beige solid. LC-MS: t_{R}=1.08 min.; [M+H]⁺: 523.38 g/mol.

### Example 8

### (R)-2′-{1-chloro-(8)-8-[2-(2,3-difluoro-4-trifluoromethyl-phenyl)-ethyl]-3-methylsulfanyl-5,6-dihydro-8H-imidazo[1,5-a]pyrazin-7-yl}-N-methyl-2′-phenyl-acetamide

Prepared according to the described general procedure (GP25) by reaction (70° C.; 96 h) of 1-chloro-8-[2-(2,3-difluoro-4-trifluoromethyl-phenyl)-ethyl]-3-methylsulfanyl-5,6,7,8-tetrahydro-imidazo[1,5-a]pyrazine (729 mg; 1.77 mmol) with toluene-4-sulfonic acid (S)-methylcarbamoyl-phenyl-methyl ester. Subsequent separation of diastereoisomers by FC (DCM/MeOH=50/1) afforded the target compound as colorless solid. LC-MS: t_{R}=1.10 min.; [M+H]⁺: 559.32 g/mol.

### Example 9

### (R)-2′-{1-chloro-(8)-8-[2-(4-difluoromethoxy-phenyl)-ethyl]-3-ethyl-5,6-dihydro-8H-imidazo[1,5-a]pyrazin-7-yl}-N-methyl-2′-phenyl-acetamide

Prepared according to the described general procedure (GP25) by reaction (70° C.; 72 h) of 1-chloro-8-[2-(4-difluoromethoxy-phenyl)-ethyl]-3-ethyl-5,6,7,8-tetrahydro-imidazo[1,5-a]pyrazine (1.530 g; 4.30 mmol) with toluene-4-sulfonic acid (S)-methylcarbamoyl-phenyl-methyl ester. Subsequent separation of diastereoisomers by FC (AcOEt/heptane=3/20 to AcOEt) afforded the target compound as yellow solid. LC-MS: t_{R}=0.93 min.; [M+H]⁺: 503.39 g/mol.

### Example 10

### (R)-2′-{1-chloro-(8)-8-[2-(4-difluoromethoxy-3-fluoro-phenyl)-ethyl]-3-ethyl-5,6-dihydro-8H-imidazo[1,5-a]pyrazin-7-yl}-N-methyl-2′-phenyl-acetamide

Prepared according to the described general procedure (GP25) by reaction (70° C.; 96 h) of 1-chloro-8-[2-(4-difluoromethoxy-3-fluoro-phenyl)-ethyl]-3-ethyl-5,6,7,8-tetrahydro-imidazo[1,5-a]pyrazine (2.260 g; 6.04 mmol) with toluene-4-sulfonic acid (S)-methylcarbamoyl-phenyl-methyl ester. Subsequent separation of diastereoisomers by FC (AcOEt/heptane=1/5 to AcOEt) afforded the target compound as pale yellow solid. LC-MS: t_{R}=0.94 min.; [M+H]⁺: 521.32 g/mol.

### Example 11

### (R)-2′-{1-chloro-(S)-8-[2-(3-difluoromethoxy-phenyl)-ethyl]-3-ethyl-5,6-dihydro-8H-imidazo[1,5-a]pyrazin-7-yl}-N-methyl-2′-phenyl-acetamide

Prepared according to the described general procedure (GP25) by reaction (70° C.; 72 h) of 1-chloro-8-[2-(3-difluoromethoxy-phenyl)-ethyl]-3-ethyl-5,6,7,8-tetrahydro-imidazo[1,5-a]pyrazine (380 mg; 1.06 mmol) with toluene-4-sulfonic acid (S)-methylcarbamoyl-phenyl-methyl ester. Subsequent separation of diastereoisomers by FC (DCM/MeOH=50/1) afforded the target compound as colorless solid. LC-MS: t_{R}=0.93 min.; [M+H]⁺: 503.42 g/mol.

### Example 12

### (R)-2′-{1-chloro-(S)-8-[2-(4-difluoromethoxy-phenyl)-ethyl]-3-methyl-5,6-dihydro-8H-imidazo[1,5-a]pyrazin-7-yl}-N-methyl-2′-phenyl-acetamide

Prepared according to the described general procedure (GP25) by reaction (70° C.; 96 h) of 1-chloro-8-[2-(4-difluoromethoxy-phenyl)-ethyl]-3-methyl-5,6,7,8-tetrahydro-imidazo[1,5-a]pyrazine (830 mg; 2.42 mmol) with toluene-4-sulfonic acid (S)-methylcarbamoyl-phenyl-methyl ester. Subsequent separation of diastereoisomers by FC (DCM/MeOH=50/1) afforded the target compound as slightly beige solid. LC-MS: t_{R}=0.92 min.; [M+H]⁺: 489.41 g/mol.

### Example 13

### N-methyl-(R)-2′-{1-methyl-3-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide and N-methyl-(R)-2′-{1-methyl-3-trifluoromethyl-(R)-8-[2-(4-trifluoromethyl-phenyl)-ethyl]-5,6-dihydro-8H-imidazo[1,5-a]pyrazin-7-yl}-2′-phenyl-acetamide

Prepared according to the described general procedure (GP25) by reaction (70° C.; 76 h) of 1-methyl-3-trifluoromethyl-8-[2-(4-trifluoromethyl-phenyl)-ethyl]-5,6,7,8-tetrahydro-imidazo[1,5-a]pyrazine (89 mg; 0.23 mmol) with toluene-4-sulfonic acid (S)-methylcarbamoyl-phenyl-methyl ester. Subsequent separation of diastereoisomers by preparative HPLC afforded the target compounds.

### Example 13a

N-methyl-(R)-2′-{1-methyl-3-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide: slightly beige solid. LC-MS: t_{R}=1.07 min.; [M+H]⁺: 525.55 g/mol.

### Example 13b

N-methyl-(R)-2′-{1-methyl-3-trifluoromethyl-(R)-8-[2-(4-trifluoromethyl-phenyl)-ethyl]-5,6-dihydro-8H-imidazo[1,5-a]pyrazin-7-yl}-2′-phenyl-acetamide: slightly beige solid. LC-MS: t_{R}=1.08 min.; [M+H]⁺: 525.53 g/mol.

### Example 14

### N-methyl-(R)-2′-{3-methylsulfanyl-1-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide

Prepared according to the described general procedure (GP25) by reaction (70° C.; 72 h) of 3-methylsulfanyl-1-trifluoromethyl-8-[2-(4-trifluoromethyl-phenyl)-ethyl]-5,6,7,8-tetrahydro-imidazo[1,5-a]pyrazine (153 mg; 0.37 mmol) with toluene-4-sulfonic acid (S)-methylcarbamoyl-phenyl-methyl ester. Subsequent separation of diastereoisomers by preparative HPLC afforded the target compound as colorless solid. LC-MS: t_{R}=1.10 min.; [M+H]⁺: 557.54 g/mol.

### Example 15

### (R)-2′-{1-iodo-3-methanesulfonyl-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 1-iodo-3-methanesulfonyl-8-[2-(4-trifluoromethyl-phenyl)-ethyl]-5,6,7,8-tetrahydro-imidazo[1,5-a]pyrazine (153 mg; 0.30 mmol) with toluene-4-sulfonic acid (S)-methylcarbamoyl-phenyl-methyl ester. Subsequent purification by preparative HPLC afforded the title compound as mixture of diastereoisomers as a colorless solid.

(R)-2′-{1-iodo-3-methanesulfonyl-(S)-8-[2-(4-trifluoromethyl-phenyl)-ethyl]-5,6-dihydro-8H-imidazo[1,5-a]pyrazin-7-yl}-N-methyl-2′-phenyl-acetamide. LC-MS: t_{R}=1.06 min.; [M+H]⁺: 647.68 g/mol.

(R)-2′-{1-iodo-3-methanesulfonyl-(R)-8-[2-(4-trifluoromethyl-phenyl)-ethyl]-5,6-dihydro-8H-imidazo[1,5-a]pyrazin-7-yl}-N-methyl-2′-phenyl-acetamide. LC-MS: t_{R}=1.07 min.; [M+H]⁺: 647.37 g/mol.

### Example 16

### (R)-2′-{3-cyclopropyl-(8)-8-[2-(3-fluoro-4-trifluoromethyl-phenyl)-ethyl]-1-trifluoromethyl-5,6-dihydro-8H-imidazo[1,5-a]pyrazin-7-yl}-N-methyl-2′-phenyl-acetamide

Prepared according to the described general procedure (GP25) by reaction (70° C.; 75 h) of 3-cyclopropyl-8-[2-(3-fluoro-4-trifluoromethyl-phenyl)-ethyl]-1-trifluoromethyl-5,6,7,8-tetrahydro-imidazo[1,5-a]pyrazine (356 mg; 0.84 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=1.07 min.; [M+H]⁺: 569.71 g/mol.

### Example 17

### (R)-2′-{3-cyclopropyl-1-ethyl-(8)-8-[2-(3-fluoro-4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 75 h) of 3-cyclopropyl-1-ethyl-8-[2-(3-fluoro-4-trifluoromethyl-phenyl)-ethyl]-5,6,7,8-tetrahydro-imidazo[1,5-a]pyrazine (170 mg; 0.44 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=0.95 min.; [M+H]⁺: 529.13 g/mol.

### Example 18

### (R)-2′-{3-cyclopropyl-(8)-8-[2-(3-fluoro-4-trifluoromethyl-phenyl)-ethyl]-1-vinyl-5,6-dihydro-8H-imidazo[1,5-a]pyrazin-7-yl}-N-methyl-2′-phenyl-acetamide

Prepared according to the described general procedure (GP25) by reaction (70° C.; 72 h) of 3-cyclopropyl-8-[2-(3-fluoro-4-trifluoromethyl-phenyl)-ethyl]-1-vinyl-5,6,7,8-tetrahydro-imidazo[1,5-a]pyrazine (91 mg; 0.24 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=0.95 min.; [M+H]⁺: 527.41 g/mol.

### Example 19

### N-methyl-(R)-2′-{3-methylsulfanyl-(S)-8-[2-(4-trifluoromethyl-phenyl)-ethyl]-1-vinyl-5,6-dihydro-8H-imidazo[1,5-a]pyrazin-7-yl}-2′-phenyl-acetamide

Prepared according to the described general procedure (GP25) by reaction (70° C.; 72 h) of 3-methylsulfanyl-8-[2-(4-trifluoromethyl-phenyl)-ethyl]-1-vinyl-5,6,7,8-tetrahydro-imidazo[1,5-a]pyrazine (620 mg; 1.68 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=0.94 min.; [M+H]⁺: 515.21 g/mol.

### Example 20

### (R)-2′-{3-ethyl-1-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 3-ethyl-1-methylsulfanyl-8-[2-(4-trifluoromethyl-phenyl)-ethyl]-5,6,7,8-tetrahydro-imidazo[1,5-a]pyrazine (340 mg; 0.92 mmol) with toluene-4-sulfonic acid (S)-methylcarbamoyl-phenyl-methyl ester. Subsequent separation of diastereoisomers by preparative HPLC afforded the target compound as off-white solid. LC-MS: t_{R}=0.91 min.; [M+H]⁺: 517.5 g/mol.

### Example 21

### (R)-2′-{3-ethoxy-1-trifluoromethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 3-ethoxy-1-trifluoromethyl-8-[2-(4-trifluoromethyl-phenyl)-ethyl]-5,6,7,8-tetrahydro-imidazo[1,5-a]pyrazine (44.6 mg; 0.10 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=1.11 min.; [M+H]⁺: 555.03 g/mol.

### Example 22

### (R)-2′-{1-chloro-3-cyclopropylmethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 1-chloro-3-cyclopropyl methyl-8-[2-(4-trifluoromethyl-phenyl)-ethyl]-5,6,7,8-tetrahydro-imidazo[1,5-a]pyrazine (230 mg; 0.59 mmol) with toluene-4-sulfonic acid (S)-methylcarbamoyl-phenyl-methyl ester. Subsequent separation of diastereoisomers by preparative HPLC afforded the target compound as slightly beige solid. LC-MS: t_{R}=0.98 min.; [M+H]⁺: 531.07 g/mol.

### Example 23

### (R)-2′-{3-ethyl-1-methanesulfonyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 3-ethyl-1-methanesulfonyl-8-[2-(4-trifluoromethyl-phenyl)-ethyl]-5,6,7,8-tetrahydro-imidazo[1,5-a]pyrazine (520 mg; 1.29 mmol) with toluene-4-sulfonic acid (S)-methylcarbamoyl-phenyl-methyl ester. Subsequent purification by FC (DCM/MeOH=50/1) afforded the target compound as colorless solid. LC-MS: t_{R}=0.99 min.; [M+H]⁺: 549.43 g/mol.

### Example 24

### (R)-2′-{3-methoxy-1-trifluoromethyl-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 72 h) of 3-methoxy-1-trifluoromethyl-8-[2-(4-trifluoromethyl-phenyl)-ethyl]-5,6,7,8-tetrahydro-imidazo[1,5-a]pyrazine (55.7 mg; 0.14 mmol) with toluene-4-sulfonic acid (S)-methylcarbamoyl-phenyl-methyl ester. Subsequent purification by preparative HPLC afforded the title compound as mixture of diastereoisomers.

(R)-2′-{3-methoxy-1-trifluoromethyl-8-[2-(4-trifluoromethyl-phenyl)-ethyl]-5,6-dihydro-8H-imidazo[1,5-a]pyrazin-7-yl}-N-methyl-2′-phenyl-acetamide: slightly beige solid. LC-MS: t_{R}=1.09 min.; [M+H]⁺: 541.99 g/mol for the (S; R)-stereoisomer; t_{R}=1.10 min.; [M+H]⁺: 541.99 g/mol for the (R; R)-stereoisomer.

### Example 25

### (R)-2′-{1-chloro-3-cyclopropyl-(S)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-chloro-3-cyclopropyl-8-[2-(4-trifluoromethyl-phenyl)-ethyl]-5,6,7,8-tetrahydro-imidazo[1,5-a]pyrazine (6.080 g; 16.44 mmol) with toluene-4-sulfonic acid (S)-methylcarbamoyl-phenyl-methyl ester. Subsequent separation of diastereoisomers by FC (DCM/MeOH=100/1) afforded the target compound as beige solid. LC-MS: t_{R}=0.98 min.; [M+H]⁺: 517.29 g/mol.

### Example 26

### (R)-2′-{1-chloro-3-cyclopropyl-(S)-8-[2-(3-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-chloro-3-cyclopropyl-8-[2-(3-trifluoromethyl-phenyl)-ethyl]-5,6,7,8-tetrahydro-imidazo[1,5-a]pyrazine (1.360 g; 3.67 mmol) with toluene-4-sulfonic acid (S)-methylcarbamoyl-phenyl-methyl ester. Subsequent separation of diastereoisomers by FC (DCM/MeOH=100/1) afforded the target compound as pale yellow solid. LC-MS: t_{R}=0.98 min.; [M+H]⁺: 517.32 g/mol.

### Example 27

### (R)-2′-{1-chloro-3-cyclopropyl-(S)-8-[2-(2-fluoro-4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-chloro-3-cyclopropyl-8-[2-(2-fluoro-4-trifluoromethyl-phenyl)-ethyl]-5,6,7,8-tetrahydro-imidazo[1,5-a]pyrazine (1.710 g; 4.40 mmol) with toluene-4-sulfonic acid (S)-methylcarbamoyl-phenyl-methyl ester. Subsequent separation of diastereoisomers by FC (DCM/MeOH=100/1) afforded the target compound as pale orange solid. LC-MS: t_{R}=1.00 min.; [M+H]⁺: 535.24 g/mol.

### Example 28

### (R)-2′-{1-ethyl-3-trifluoromethyl-(8)-8-[2-(4-trifluoromethyl-phenyl)-ethyl]-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-ethyl-3-trifluoromethyl-8-[2-(4-trifluoromethyl-phenyl)-ethyl]-5,6,7,8-tetrahydro-imidazo[1,5-a]pyrazine (740 mg; 1.89 mmol) with toluene-4-sulfonic acid (S)-methylcarbamoyl-phenyl-methyl ester. Subsequent separation of diastereoisomers by FC (DCM/MeOH=100/1) afforded the target compound as pale yellow solid. LC-MS: t_{R}=1.09 min.; [M+H]⁺: 539.12 g/mol.

### Example 29

### (R)-2′-{1-ethyl-(8)-8-[2-(3-fluoro-4-trifluoromethyl-phenyl)-ethyl]-3-trifluoromethyl-5,6-dihydro-8H-imidazo[1,5-a]pyrazin-7-yl}-N-methyl-2′-phenyl-acetamide

Prepared according to the described general procedure (GP25) by reaction (70° C.; 4 days) of 1-ethyl-8-[2-(3-fluoro-4-trifluoromethyl-phenyl)-ethyl]-3-trifluoromethyl-5,6,7,8-tetrahydro-imidazo[1,5-a]pyrazine (200 mg; 0.48 mmol) with toluene-4-sulfonic acid (S)-methylcarbamoyl-phenyl-methyl ester. Subsequent separation of diastereoisomers by preparative HPLC afforded the target compound as beige solid. LC-MS: t_{R}=1.10 min.; [M+H]⁺: 557.51 g/mol.

### II. BIOLOGICAL ASSAYS

### In Vitro Assay

The orexin receptor antagonistic activity of the compounds of formula (I) is determined in accordance with the following experimental method.

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 U/ml penicillin, 100 μg/ml streptomycin and 10% heat inactivated fetal calf serum (FCS). The cells are seeded at 20′000 cells/well into 384-well black clear bottom sterile plates (Greiner). The seeded plates are incubated overnight at 37° C. in 5% CO₂.

Human orexin-A as an agonist is prepared as 1 mM stock solution in MeOH:water (1:1), diluted in HBSS containing 0.1% bovine serum albumin (BSA), NaHCO₃: 0.375 g/l and 20 mM HEPES for use in the assay at a final concentration of 3 nM.

Antagonists are prepared as 10 mM stock solution in DMSO, then diluted in 384-well plates using DMSO followed by a transfer of the dilutions into in HBSS containing 0.1% bovine serum albumin (BSA), NaHCO₃: 0.375 g/l and 20 mM HEPES. On the day of the assay, 50 μl of staining buffer (HBSS containing 1% FCS, 20 mM HEPES, NaHCO₃: 0.375 g/l, 5 mM probenecid (Sigma) and 3 μM of the fluorescent calcium indicator fluo-4 AM (1 mM stock solution in DMSO, containing 10% pluronic) is added to each well. The 384-well cell-plates are incubated for 50 min at 37° C. in 5% CO₂ followed by equilibration at RT for 30-120 min before measurement.

Within the Fluorescent Imaging Plate Reader (FLIPR Tetra, Molecular Devices), antagonists are added to the plate in a volume of 10 Owen, incubated for 10 min and finally 10 Owen of agonist is added. Fluorescence is measured for each well at 1 second intervals, and the height of each fluorescence peak is compared to the height of the fluorescence peak induced by 3 nM orexin-A with vehicle in place of antagonist. For each antagonist, the IC₅₀ value (the concentration of compound needed to inhibit 50% of the agonistic response) is determined and may be normalized using the obtained IC₅₀ value of a on-plate reference compound (normalized values in Table 1 indicated by an asterisk*).

The calculated IC₅₀ values of the compounds may fluctuate depending on the daily cellular assay performance. Fluctuations of this kind are known to those skilled in the art.

With respect to the OX₁ receptor, IC₅₀ values of 28 exemplified compounds are in the range of 12-3539 nM with an average of 487 nM; The IC₅₀ values of 2 compounds have been measured >10000 nM. With respect to the OX₂ receptor, IC₅₀ values of all exemplified compounds are in the range of 1-1206 nM with an average of 90 nM. Antagonistic activities of selected compounds are displayed in Table 1.

TABLE 1
Antagonistic activities of compounds with
respect to OX₁ and OX₂ receptors.

| Compound of Example | OX₁ IC₅₀ (in nM) | OX₂ IC₅₀ (in nM) |
|---|---|---|
| 3 | 43 | 11 |
| 8 | 56 | 21 |
| 11 | 45 | 2 |
| 13a | 259 *² | 8 *² |
| 17 | 384 * *² | 8 * *² |
| 21 | 3539 * | 63 * |
| 23 | 1677 * *² | 25 * *² |
| 26 | 91 * | 7 * |
| 28 | 505 * | 13 * |

* normalized values as described above
*² geometric mean of n = 2 values

1. A compound of formula (I), 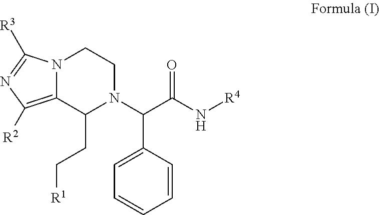
wherein
R⁴ represents (C₁₋₄)alkyl; and
R¹, R², and R³ represent one of the following combinations:
R³ represents cyclopropyl;
R² represents halogen, trifluoromethyl, (C₁₋₄)alkyl, or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₃₋₆)cycloalkyl-(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —SO₂—(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents halogen, trifluoromethyl, or vinyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents —S{O}ₙ—(C₁₋₄)alkyl, wherein n represents the integer 0 or 2; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkoxy;
R² represents trifluoromethyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents trifluoromethyl;
R² represents (C₁₋₄)alkyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl;
in free or salt form.

2. The compound of formula (I) according to claim 1, which is also a compound of formula (II), wherein the absolute configuration is [(R)-2′; (S)-8]: 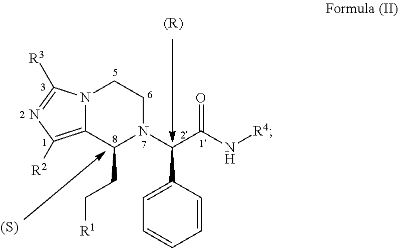
in free or salt form.

3. The compound according to claim 1, wherein R⁴ represents methyl;
in free or salt form.

4. The compound according to claim 1, wherein R¹, R², and R³ represent one of the following combinations:
R³ represents cyclopropyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents halogen; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents (C₁₋₄)alkyl;
R² represents halogen; and
R¹ represents a phenyl group, which group is mono-, di-, or tri-substituted, wherein one substituent is difluoromethoxy, and the remaining substituents (if present) are independently selected from the group consisting of (C₁₋₄)alkyl, (C₁₋₄)alkoxy, halogen, cyano, difluoromethoxy, trifluoromethoxy and trifluoromethyl;
in free or salt form.

5. The compound according to claim 1, wherein R¹, R², and R³ represent one of the following combinations:
R³ represents cyclopropyl;
R² represents trifluoromethyl or (C₁₋₄)alkyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents —S—(C₁₋₄)alkyl;
R² represents trifluoromethyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
or
R³ represents trifluoromethyl;
R² represents (C₁₋₄)alkyl; and
R¹ represents a phenyl group, which group is mono-, di-, or tri-substituted, wherein the substituents are independently selected from the group consisting of (C₁₋₄)alkyl, (C₁₋₄)alkoxy, halogen, cyano, difluoromethoxy, trifluoromethoxy and trifluoromethyl;
in free or salt form.

6. The compound according to claim 1, wherein, if not explicitly stated otherwise, R¹ represents a phenyl group, which is mono-, di-, or tri-substituted, wherein one substituent is difluoromethoxy, trifluoromethoxy or trifluoromethyl, and the remaining substituents (if present) are fluorine;
in free or salt form.

7. The compound according to claim 1, wherein R¹ represents a phenyl group, which group is mono-, di-, or tri-substituted, wherein one substituent is difluoromethoxy, and the remaining substituents (if present) are fluorine;
in free or salt form.

8. The compound according to claim 1 selected from the group consisting of:
(R)-2′-{1-chloro-3-cyclopropyl-(S)-8-[2-(4-fluoro-3-trifluoromethyl-phenyl)-ethyl]-5,6-dihydro-8H-imidazo[1,5-a]pyrazin-7-yl}-N-methyl-2′-phenyl-acetamide;
(R)-2′-{1-chloro-(S)-8-[2-(4-chloro-3-fluoro-phenyl)-ethyl]-3-cyclopropyl-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(4-difluoromethoxy-phenyl)-ethyl]-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(3-fluoro-4-trifluoromethoxy-phenyl)-ethyl]-5,6-dihydro-8H-imidazo[1,5-a]pyrazin-7-yl}-N-methyl-2′-phenyl-acetamide:
(R)-2′-{1-chloro-3-cyclopropyl-(S)-8-[2-(4-difluoromethoxy-3-fluoro-phenyl)-ethyl]-5,6-dihydro-8H-imidazo[1,5-a]pyrazin-7-yl}-N-methyl-2′-phenyl-acetamide;
(R)-2′-{1-chloro-3-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{1-chloro-(S)-8-[2-(2,3-difluoro-4-trifluoromethyl-phenyl)-ethyl]-3-methylsulfanyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-3-fluoro-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(3-difluoromethoxy-phenyl)-ethyl]-3-ethyl-5,6-dihydro-8H-imidazo[1,5-a]pyrazin-7-yl}-N-methyl-2′-phenyl-acetamide;
(R)-2′-{1-chloro-(S)-8-[2-(4-difluoromethoxy-phenyl)-ethyl]-3-methyl-5,6-dihydro-8H-imidazo[1,5-a]pyrazin-7-yl}-N-methyl-2′-phenyl-acetamide;
N-methyl-(R)-2′-{1-methyl-3-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide;
N-methyl-(R)-2′-{3-methylsulfanyl-1-trifluoromethyl-(S)-8-[2-(4-trifluoromethyl-phenyl)-ethyl]-5,6-dihydro-8H-imidazo[1,5-a]pyrazin-7-yl}-2′-phenyl-acetamide;
(R)-2′-{3-cyclopropyl-(S)-8-[2-(3-fluoro-4-trifluoromethyl-phenyl)-ethyl]-1-trifluoromethyl-5,6-dihydro-8H-imidazo[1,5-a]pyrazin-7-yl}-N-methyl-2′-phenyl-acetamide;
(R)-2′-{3-cyclopropyl-1-ethyl-(S)-8-[2-(3-fluoro-4-trifluoromethyl-phenyl)-ethyl]-5,6-dihydro-8H-imidazo[1,5-a]pyrazin-7-yl}-N-methyl-2′-phenyl-acetamide;
(R)-2′-{3-cyclopropyl-(S)-8-[2-(3-fluoro-4-trifluoromethyl-phenyl)-ethyl]-1-vinyl-5,6-dihydro-8H-imidazo[1,5-a]pyrazin-7-yl}-N-methyl-2′-phenyl-acetamide;
N-methyl-(R)-2′-{3-methylsulfanyl-(S)-8-[2-(4-trifluoromethyl-phenyl)-ethyl]-1-vinyl-5,6-dihydro-8H-imidazo[1,5-a]pyrazin-7-yl}-2′-phenyl-acetamide;
(R)-2′-{3-ethyl-1-methylsulfanyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{3-ethoxy-1-trifluoromethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide; and
(R)-2′-{3-ethyl-1-methanesulfonyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
in free or salt form.

9. The compound according to claim 1 selected from the group consisting of:
(R)-2′-{1-ethyl-(S)-8-[2-(3-fluoro-4-trifluoromethyl-phenyl)-ethyl]-3-trifluoromethyl-5,6-dihydro-8H-imidazo[1,5-a]pyrazin-7-yl}-N-methyl-2′-phenyl-acetamide;
(R)-2′-{1-chloro-3-cyclopropyl-(S)-8-[2-(4-trifluoromethyl-phenyl)-ethyl]-5,6-dihydro-8H-imidazo[1,5-a]pyrazin-7-yl}-N-methyl-2′-phenyl-acetamide;
(R)-2′-{1-ethyl-3-trifluoromethyl-(S)-8-[2-(4-trifluoromethyl-phenyl)-ethyl]-5,6-dihydro-8H-imidazo[1,5-a]pyrazin-7-yl}-N-methyl-2′-phenyl-acetamide:
(R)-2′-{1-chloro-3-cyclopropyl-(S)-8-[2-(3-trifluoromethyl-phenyl)-ethyl]-5,6-dihydro-8H-imidazo[1,5-a]pyrazin-7-yl}-N-methyl-2′-phenyl-acetamide; and
(R)-2′-{1-chloro-3-cyclopropyl-(S)-8-[2-(2-fluoro-4-trifluoromethyl-phenyl)-ethyl]-5,6-dihydro-8H-imidazo[1,5-a]pyrazin-7-yl}-N-methyl-2′-phenyl-acetamide;
in free or salt form.

10. A pharmaceutical composition containing, as active principle, the compound of formula (I) according to claim 1, in free or pharmaceutically acceptable salt form, and at least one therapeutically inert excipient.

11. (canceled)

12. A method for the prevention or treatment of a disease selected from the group consisting of all types of sleep disorders, of stress-related syndromes, of addictions, of cognitive dysfunctions in the healthy population and in psychiatric and neurologic disorders, of eating or drinking disorders, comprising administering to a subject a pharmaceutically active amount of the compound according to claim 1 in free or pharmaceutically acceptable salt form.

13. (canceled)

14. The method according to claim 12, wherein said compound is a compound according to claim 2 in free or pharmaceutically acceptable salt form.

15. The method according to claim 12, wherein said compound is a compound according to claim 8 in free or pharmaceutically acceptable salt form.

16. The method according to claim 12, wherein said compound is a compound according to claim 9 in free or pharmaceutically acceptable salt form.

17. The pharmaceutical composition according to claim 10, wherein said compound is a compound according to claim 2 in free or pharmaceutically acceptable salt form.

18. The pharmaceutical composition according to claim 10, wherein said compound is a compound according to claim 8 in free or pharmaceutically acceptable salt form.

19. The pharmaceutical composition according to claim 10, wherein said compound is a compound according to claim 9 in free or pharmaceutically acceptable salt form.
